(19) United States
(12) Patent Application Publication
(10) Pub. No.: US 2020/0185063 A1
(43) Pub. Date: Jun. 11, 2020

(54) SYSTEMS AND METHODS FOR PATIENT STRATIFICATION AND IDENTIFICATION OF POTENTIAL BIOMARKERS

(71) Applicant: Berg LLC, Framingham, MA (US)

(72) Inventors: Niven Rajin Narain, Cambridge, MA (US); Viatcheslav R. Akmaev, Sudbury, MA (US); Leonardo Rodrigues, Ashland, MA (US); Gregory Mark Miller, Natick, MA (US)

(21) Appl. No.: 16/307,406

(22) PCT Filed: Jun. 5, 2017
(86) PCT No.: PCT/US2017/036020

§ 371 Date: Dec. 5, 2018

Related U.S. Application Data
(60) Provisional application No. 62/345,858, filed on Jun. 5, 2016.

(51) Int. Cl. G16B 50/30; G16B 20/00; G16H 50/50; G16H 50/70; G16B 25/10; G16B 40/00; G16B 45/00

(52) U.S. Cl. CPC G16B 50/30; G16B 20/00; G16H 50/50; G16B 45/00; G16B 25/10; G16B 40/00; G16H 50/70

(57) ABSTRACT

Disclosed herein are methods and systems for identifying one or more potential biomarkers for a clinical outcome related to administration of an agent. The method includes processing molecular profile data for a plurality of subjects where the molecular profile data includes data obtained before, during and/or after administration of an agent to the plurality of subjects. The method also includes processing clinical records data for the subjects, where the clinical records data includes clinical outcome data, integrating the processed molecular profile data and the processed clinical records data for the subjects and storing in a database as merged data, selecting two or more subsets of the merged data using one or more criteria based on the clinical records data to generate two or more selected data sets, and analyzing one or more of the selected data sets to identify one or more potential biomarkers for a clinical outcome related to administration of the agent.

### RELATED APPLICATION

This application is a 35 U.S.C. § 371 national stage filing of International Application No. PCT/US2017/036020, filed on Jun. 5, 2017, which in turn claims benefit of and priority to U.S. Provisional Application No. 62/345,858, filed on Jun. 5, 2016. The entire contents of which is each of the foregoing applications are incorporated herein by reference herein in their entirety.

### BACKGROUND

Many systems analyze data to gain insights into various aspects of healthcare, including patient response to a particular therapy. Insights can be gained by determining relationships among healthcare data gathered from patients. Conventional methods predetermine a few relevant variables to extract from healthcare data for processing and analysis. Based on the few pre-selected variables, relationships are established between various factors such as medical drug, disease, symptoms, etc. Preselecting the variables to be analyzed limits the ability to discover new or unknown relationships. Preselecting the variables also limits the ability to discover other relevant variables. For example, if the variables are preselected when considering analysis of diabetes, one would be limited to examining variables known or suspected to be relevant to diabetes and may overlook another variable relevant to diabetes that was previously unknown to the healthcare community.

Instead of focusing on preselected variables, a preferred method would be to analyze medical data to identify novel relationships among the data that could facilitate identification of biomarkers for use in patient therapy. For example, clinical trials provide an opportunity for collecting large amounts of medical data through a detailed analysis of patient response to a particular therapy. However, the challenge has been to analyze these large amounts of data in a way that identifies key drivers of patient response. Therefore a need exists for a method of integrating large amounts of medical data to determine novel relationships among the data, and ultimately to identify biological markers to facilitate patient therapy.

### SUMMARY

Embodiments described herein provide methods and systems for identification of one or more biomarkers or potential biomarkers for a clinical outcome related to administration of an agent. Some embodiments provide methods and systems for patient stratification. Some embodiments may be employed in connection with a clinical trial.

An embodiment of the invention provides a method including processing molecular profile data for each subject in a plurality of subjects, processing clinical records data for each of the plurality of subjects, integrating the processed molecular profile data and the processed clinical records data for the plurality of subjects and storing in a database as merged data, selecting two or more subsets of the merged data using one or more criteria based on the clinical records data to generate two or more selected data sets, a analyzing one or more of the selected data sets to identify one or more potential biomarkers for a clinical outcome related to administration of the agent. The molecular profile data for each subject includes one or more of proteomics, metabolomics, lipidomics, genomics, transcriptomics, microarray and sequencing data generated from analysis of a plurality of samples obtained from the subject. The plurality of samples for each subject includes samples obtained before, during, and/or after administration of an agent to the subject. The clinical records data for each subject includes data based on one or both of samples obtained from the subject and measurements made of the subject before, during, and/or after administration of the agent. The clinical records data includes clinical outcome data.

In some embodiments, the method also includes administering the agent to the plurality of subjects. In some embodiments, the method also includes, for each subject, analyzing the plurality of samples obtained from the subject to obtain the molecular profile data.

In some embodiments, the clinical records data further includes one or more of pharmacokinetics data, medical history data, laboratory test data, and data from a mobile wearable device. In some embodiments, the clinical records data for a subject further includes demographic information regarding the subject.

In some embodiments, the one or more selected data sets are analyzed using one or more of statistical methods, machine learning methods, and artificial intelligence methods to identify the one or more potential biomarkers for the clinical outcome related to administration of the agent. In some embodiments, the one or more selected data sets are analyzed using two or more of statistical methods, machine learning methods, and artificial intelligence methods to identify the one or more potential biomarkers for the clinical outcome related to administration of the agent.

In some embodiments, analyzing one or more of the selected data sets to identify the one or more potential biomarkers for the clinical outcome related to administration of the agent includes: generating one or more causal relationship networks based on one or more of the selected data sets; and analyzing the generated one or more causal relationship networks to identify nodes corresponding to one or more outcome drivers. In some embodiments, analyzing the generated causal relationship networks to identify nodes corresponding to the one or more outcome drivers includes identifying as outcome drivers variables corresponding to nodes connected to the clinical outcome in one or more of the generated causal relationship networks by relationships having a degree of connection equal to or less than n. In some embodiments, n is 10 or 9 or 8 or 7 or 6 or 5 or 4 or 3 or 2 or 1. In some embodiments, n is 3 or 2 or 1. In some embodiments, n is 2 or 1. In some embodiments, n is 1. In some embodiments, analyzing the generated causal relationship networks to identify nodes corresponding to the one or more outcome drivers includes analysis of network topology features of the one or more generated causal relationship networks.

In some embodiments, the generated two or more selected data sets include a first plurality of selected data sets each corresponding to a subject that exhibited the clinical outcome and a second plurality of selected data sets each corresponding to a subject that did not exhibit the first clinical outcome, and generating the one or more causal relationship networks based on one or more of the selected data sets includes: generating a first plurality of causal relationship networks each based on one of the first plurality of selected data sets corresponding to subjects that exhibited the clinical outcome, and generating a second plurality of causal relationship networks each based on one of the second plurality of selected data sets corresponding to subjects that did not exhibit the clinical outcome. Analyzing the generated causal relationship networks to identify nodes corresponding to one or more outcome drivers includes: identifying one or more first commonalities among first plurality of causal relationship networks, identifying one or more second commonalities among the second plurality of causal relationship networks, and comparing the first commonalities and the second commonalities to identify the one or more outcome drivers in accordance with some embodiments.

In some embodiments, the generated two or more selected data sets include a first selected data set including data corresponding to one or more subjects that exhibited the clinical outcome and a second selected data set including data corresponding to one or more subjects that did not exhibit the clinical outcome, and generating the one or more causal relationship networks based on at least some of the selected data sets includes: generating a first causal relationship network based on the first selected data set corresponding to subjects that exhibited the clinical outcome, and generating a second causal relationship network based on the second selected data set corresponding to subject that did not exhibit the clinical outcome. The one or more outcome drivers are identified based on a comparison of the first causal relationship network to the second causal relationship network in accordance with some embodiments. In some embodiments, the comparison of the first causal relationship network to the second causal relationship network includes generation of a differential causal relationship from the first causal relationship network and the second causal relationship network, and the one or more outcome drivers are identified from the generated differential causal relationship network.

In some embodiments, the generated causal relationship networks are Bayesian causal relationship networks. In some embodiments, the one or more outcome drivers are the one or more biomarkers or potential biomarkers for the clinical outcome related to administration of the agent.

In some embodiments, the generated two or more selected data sets includes a first selected data set including data from subjects that exhibited the clinical outcome and a second sliced data including to data from subjects that did not exhibit the clinical outcome; and analyzing one or more of the selected data sets to identify one or more potential biomarkers for a clinical outcome related to administration of the agent further includes identifying one or more variables differentially expressed between first selected data set and the second selected data set at a statistically significant level. In some embodiments, the first selected data set and the second selected data set correspond to the same time point or the same range of time points relative to a time of administration of an agent. In some embodiments, identifying the one or more variables differentially expressed between first selected data set and the second selected data set at a statistically significant level includes employing a two-sample t-test or limma methodology. In some embodiments, identifying the one or more variables differentially expressed between first selected data set and the second selected data set at a statistically significant level includes performing a regression analysis.

In some embodiments, analyzing one or more of the selected data sets to identify one or more potential biomarkers for a clinical outcome related to administration of the agent also includes employing machine learning to analyze the identified outcome drivers and the one or more differentially expressed variables as possible biomarkers and, based on the analysis, selecting a subset of the possible biomarkers as the one or more potential biomarkers, wherein the machine learning penalizes possible biomarkers that are strongly correlated with other possible biomarkers and rewards possible biomarkers based on a level of correlation with the clinical outcome, thereby identifying one or more potential biomarkers for the clinical outcome. In some embodiments, the machine learning employed to analyze the possible biomarkers applies logistic regression with the elastic net penalty.

In some embodiments, integrating the processed molecular profile data and the processed clinical records data for the plurality of subjects and storing in the database as merged data comprises storing the merged data in a master file that includes a subject identification and a time associated with each sample. In some embodiments, linear interpolation is used to determine interpolated values of at least some clinical records data at times corresponding to those associated with molecular profile samples.

In some embodiments, the method also includes generating an in silico computational diagnostic patient map for determination of a subject response from analysis of topological features of the generated Bayesian causal relationship networks. In some embodiments, the method also includes the in silico computational diagnostic patient map for patient stratification.

In some embodiments, one or more potential biomarkers are potential biomarkers for agent efficacy or for an adverse event. In some embodiments, the method is a method for identifying one or more potential biomarkers for efficacy of the agent in treatment of a disease or a disorder. In some embodiments, the method is a method for identifying one or more potential biomarkers for the occurrence of an adverse event related to administration of the agent. In some embodiments, the method is a method for patient stratification, and the method also includes employing the one or more potential biomarkers for patient stratification.

In some embodiments, the one or more potential biomarkers are employed for patient stratification to determine whether or not to treat a patient using the agent. In some embodiments, the method is a method for patient stratification.

In some embodiments, the administration of an agent to the plurality of subjects occurs during a clinical trial for the agent, and the method also in includes employing the identified one or more potential biomarkers for patient stratification during a subsequent clinical trial of the agent or during a subsequent stage of the same clinical trial of the agent. In some embodiments, the one or more potential biomarkers are used for patient stratification to determine which patients are enrolled in the subsequent clinical trial. In some embodiments, the one or more potential biomarkers are used for patient stratification to determine the patients that receive the agent in the subsequent clinical trial.

In some embodiments, the one or more criteria for selecting two or more subsets of the merged data includes a phenotypic classification. In some embodiments, the one or more criteria for selecting two or more subsets of the merged data comprises clinical outcome data.

In some embodiments, the one or more criteria for selecting two or more subsets of the merged data includes data regarding whether a subject experienced an adverse event during or after administration of the agent.

In some embodiments, the agent is intended for treatment of a disease or disorder and the one or more criteria for selecting two or more subsets of the merged data includes data regarding responsiveness of the subject to the treatment.

In some embodiments, the selected two or more subsets of the merged data include a selected data set for each individual subject. In some embodiments, the two or more selected data sets comprise a selected data set including the merged data from all of the plurality of subjects. In some embodiments, the one or more samples for each subject comprise one or more of blood, tissue, and urine samples. In some embodiments, the one or more samples for each subject comprise two or more of blood, plasma, tissue, and urine samples.

In some embodiments, the molecular profile data for each subject comprises two or more of proteomics, metabolomics, lipidomics, genomics, transcriptomics, microarray and sequencing data. In some embodiments, the molecular profile data for each subject comprises three or more of proteomics, metabolomics, lipidomics, genomics, transcriptomics, microarray and sequencing data. In some embodiments, the molecular profile data for each subject comprises proteomics, metabolomics, and lipidomics data. In some embodiments, the molecular profile data for each subject further includes one or more of genomics, transcriptomics, microarray and sequencing data.

In some embodiments, the clinical outcome data comprises data regarding a state or status of a disease or a disorder. In some embodiments, the agent is an agent for treatment of a disease or disorder and wherein the clinical outcome data includes data indicating whether a subject was responsive or refractory in response to treatment with the agent. In some embodiments, the clinical outcome data comprises data regarding an adverse event occurring during or after administration of the agent.

In some embodiments, the method also includes processing the merged data by reconciling duplicated clinical records data and resolving discrepancies. In some embodiments, the method also includes filtering the merged data to remove molecular data for which corresponding clinical records data is missing. In some embodiments, the processing molecular profile data for each subject also includes: merging the molecular profile data collected at different time points over the course of the treatment for the plurality of subjects; filtering the molecular profile data to remove infrequently measured variables; normalizing the molecular profile data; and imputing any variable not measured for a particular subject of the plurality of subjects.

In some embodiments, the agent is intended for treatment of cancer. In some embodiments, the clinical outcome data includes tumor size measurements. In some embodiments, the clinical outcome data comprises data from functional imaging of a tumor.

In some embodiments, analyzing one or more of the selected data sets to identify one or more potential biomarkers for a clinical outcome related to administration of the agent includes generating a Bayesian causal relationship network for each of the one or more selected data sets. The method further includes comparing the generated Bayesian causal relationship networks from selected data sets from subjects with a Bayesian causal relationship network generated based on data obtained from an in vitro model of cancer in accordance with some embodiments.

In some embodiments, the method also includes generating a subject-specific profile that includes a graphical representation of demographic information for the subject; and a graphical representation of outcome information for the subject. In some embodiments, the graphical representation of outcome information for the subject includes: a graphical representation of adverse event information for the subject; and a graphical representation of information regarding responsivity to the agent.

In some embodiments, some or all of the subjects in the plurality of subjects are afflicted with a disorder. In some embodiments, the disorder is selected from the group consisting of cancer, diabetes and cardiovascular disease. In some embodiments, the disorder is a cancer. In some embodiments, the cancer includes a solid tumor.

In some embodiments, for each subject, the clinical records data includes pharmacokinetic data from samples obtained at the same time points as samples for molecular profile data were obtained. In some embodiments, the method further includes, for each patient, obtaining the plurality of samples for molecular profile data at a plurality of time points and obtaining samples for pharmacokinetic data at the same plurality of time points.

In some embodiments, the identified one or more potential biomarkers are one or more biomarkers for the clinical outcome related to administration of the agent. In some embodiments, the method is a method of identifying one or more biomarkers for the clinical outcome related to administration of the agent.

Another embodiments provides a system including: a database; a memory; and a processor in communication with the memory. The processor includes an omics module, a clinical records module, an integration module, a slicing module, and an analysis module. The omics module is configured to process molecular profile data for each subject in a plurality of subjects, the molecular profile data for each subject comprising one or more of proteomics, metabolomics, lipidomics, genomics, transcriptomics, microarray and sequencing data generated from analysis of a plurality of samples obtained from the subject, the plurality of samples for each subject including samples obtained before, during, and/or after administration of an agent to the subject. The clinical records module is configured to process clinical records data for each of the plurality of subjects, the clinical records data for each subject including data based on one or both of samples obtained from the subject and measurements made of the subject before, during, and/or after administration of the agent, the clinical records data comprising clinical outcome data. The an integration module is configured to integrate the processed molecular profile data and the processed clinical records data for the plurality of subjects and storing in the database as merged data. The slicing module is configured to select two or more subsets of the merged data using one or more criteria based on the clinical records data to generate two or more selected data sets. The analysis module is configured to analyze one or more of the selected data sets to identify one or more potential biomarkers for a clinical outcome related to administration of the agent.

In some embodiments, the processor is configured to, for each subject, analyze the plurality of samples obtained from the subject to obtain the molecular profile data. In some embodiments, the clinical records data further includes one or more of pharmacokinetics data, medical history data, laboratory test data, and data from a mobile wearable device. In some embodiments, the clinical records data for a subject further comprises demographic information regarding the subject. In some embodiments, the one or more selected data sets are analyzed using one or more of statistical methods, machine learning methods, and artificial intelligence methods to identify the one or more potential biomarkers for the clinical outcome related to administration of the agent. In some embodiments, the one or more selected data sets are analyzed using two or more of statistical methods, machine learning methods, and artificial intelligence methods to identify the one or more potential biomarkers for the clinical outcome related to administration of the agent.

In some embodiments, the analysis module is further configured to: generate one or more causal relationship networks based on one or more of the selected data sets; and analyze the generated one or more causal relationship networks to identify nodes corresponding to one or more outcome drivers.

In some embodiments, the analysis module is configured to analyze the generated causal relationship networks to identify nodes corresponding to the one or more outcome drivers includes identifying as outcome drivers variables corresponding to nodes connected to the clinical outcome in one or more of the generated causal relationship networks by relationships having a degree of connection equal to or less than n, where n is 6, 5, 4, 3, 2 or 1.

In some embodiments, the analysis module is further configured to employ machine learning to analyze the identified outcome drivers and the one or more differentially expressed variables as possible biomarkers and, based on the analysis, selecting a subset of the possible biomarkers as the one or more potential biomarkers, wherein the machine learning penalizes possible biomarkers that are strongly correlated with other possible biomarkers and rewards possible biomarkers based on a level of correlation with the clinical outcome, thereby identifying one or more potential biomarkers for the clinical outcome. In some embodiments, the machine learning employed analyzes the possible biomarkers applies logistic regression with the elastic net penalty.

In some embodiments, the integration module is configured to integrate the processed molecular profile data and the processed clinical records data for the plurality of subjects and storing in the database as merged data, and store the merged data in a master file that includes a subject identification and a time associated with each sample.

In some embodiments, the processor is further configured to: generate an in silico computational diagnostic patient map for determination of a subject response from analysis of topological features of the generated Bayesian causal relationship networks. In some embodiments, the in silico computational diagnostic map is configured for use in patient stratification.

In some embodiments, the system is a system for identifying one or more potential biomarkers for efficacy of the agent in treatment of a disease or a disorder. In some embodiments, the system is a system for identifying one or more potential biomarkers for the occurrence of an adverse event related to administration of the agent. In some embodiments, the system is a system for patient stratification; and wherein the method further comprises employing the one or more potential biomarkers for patient stratification.

In some embodiments, the system is a system for patient stratification; the administration of an agent to the plurality of subjects occurs during a clinical trial for the agent; and the processor is further configured to employ the identified one or more potential biomarkers for patient stratification during a subsequent clinical trial of the agent or during a subsequent stage of the same clinical trial of the agent. The system of any one of the preceding claims, wherein the two or more selected data sets comprise a selected data set for each individual subject.

In some embodiments, the processor is further configured to: process the merged data by reconciling duplicated clinical records data and resolving discrepancies. In some embodiments, the processor is further configured to: filter the merged data to remove molecular data for which corresponding clinical records data is missing.

In some embodiments, the omics module is further configured to: merge the molecular profile data collected at different time points over the course of the treatment for the plurality of subjects; filter the molecular profile data to remove infrequently measured variables; normalize the molecular profile data; and impute any variable not measured for a particular subject of the plurality of subjects.

Another embodiments provides a non-transitory computer readable medium storing instructions that when executed causes a processing device to implement any of the methods disclosed or described herein.

The present invention is also based, at least in part, on the discovery that the biomarker PDIA3 is expressed at a higher than average level in subjects that are clinically responsive to treatment of cancer with Coenzyme Q10 (CoQ10), and is expressed at a lower than average level in subjects that are refractory to the treatment of cancer with CoQ10. Accordingly, the present invention provides methods for predicting the response of a subject having cancer to treatment with CoQ10, or selecting a subject with cancer as a good candidate for treatment of the cancer with CoQ10.

In one aspect, the present invention provides methods for selecting a subject for treatment of a cancer with CoQ10, comprising: (a) detecting the level of PDIA3 in a biological sample of the subject, and (b) comparing the level of PDIA3 in the biological sample with a predetermined threshold value, wherein the subject is selected for treatment of a cancer with CoQ10 if the level of PDIA3 is above the predetermined threshold value.

In another aspect, the present invention provides methods for predicting whether a subject having a cancer will respond to treatment with CoQ10, comprising: (a) detecting the level of PDIA3 in a biological sample of the subject, and (b) comparing the level of PDIA3 in the biological sample with a predetermined threshold value, wherein a level of PDIA3 above the predetermined threshold value indicates the subject is likely to respond to treatment of a cancer with CoQ10.

In certain embodiments, the biological sample is selected from the group consisting of blood, serum, urine, organ tissue, biopsy tissue, feces, skin, hair, and cheek tissue.

In other embodiments, detecting the level of PDIA3 in a biological sample of the subject, comprises determining the amount of PDIA3 protein in the biological sample. In one embodiment, the level of PDIA3 protein is determined by immunoassay or ELISA. In another embodiment, the level of PDIA3 protein is determined by mass spectrometry.

In one embodiment, detecting the level of PDIA3 in a biological sample of the subject comprises contacting the biological sample with a reagent that selectively binds to the PDIA3 to form a biomarker complex, and detecting the biomarker complex. In one embodiment, the reagent is an anti-PDIA3 antibody that selectively binds to at least one epitope of PDIA3.

In another embodiment, detecting the level of PDIA3 in a biological sample of the subject comprises determining the amount of PDIA3 mRNA in the biological sample. In one embodiment, an amplification reaction is used for determining the amount of PDIA3 mRNA in the biological sample. In another embodiment, the amplification reaction is a polymerase chain reaction (PCR); a nucleic acid sequence-based amplification assay (NASBA); a transcription mediated amplification (TMA); a ligase chain reaction (LCR); or a strand displacement amplification (SDA).

In one embodiment, a hybridization assay is used for determining the amount of PDIA3 mRNA in the biological sample. In certain embodiments, an oligonucleotide that is complementary to a portion of a PDIA3 mRNA is used in the hybridization assay to detect the PDIA3 mRNA.

In a further aspect, the present invention provides methods for selecting a subject for treatment of a cancer with CoQ10, comprising: (a) contacting a biological sample with a reagent that selectively binds to PDIA3; (b) allowing a complex to form between the reagent and PDIA3; (c) detecting the level of the complex, and (d) comparing the level of the complex with a predetermined threshold value, wherein the subject is selected for treatment of a cancer with CoQ10 if the level of the complex is above the predetermined threshold value.

In another aspect, the present invention provides methods for predicting whether a subject having a cancer will respond to treatment with Coenzyme Q10 (CoQ10), comprising: (a) contacting a biological sample with a reagent that selectively binds to PDIA3; (b) allowing a complex to form between the reagent and PDIA3; (c) detecting the level of the complex, and (d) comparing the level of the complex with a predetermined threshold value, wherein a level of PDIA3 above the predetermined threshold value indicates the subject is likely to respond to treatment of a cancer with CoQ10.

In one embodiment, the reagent is an anti-PDIA3 antibody. In another embodiment, the antibody comprises a detectable label. In still another embodiment, the step of detecting the level of the complex further comprises contacting the complex with a detectable secondary antibody and measuring the level of the secondary antibody.

In certain embodiments, the biological sample is selected from the group consisting of blood, serum, urine, organ tissue, biopsy tissue, feces, skin, hair, and cheek tissue.

In other embodiments, the level of the complex is detected by immunoassay or ELISA.

In some embodiments the cancer is a solid tumor. In other embodiments, the cancer is selected from the group consisting of squamous cell carcinoma, glioblastoma, and pancreatic cancer.

In certain embodiments, the methods of the invention further comprising administering CoQ10 to the subject where the level of PDIA3 above the predetermined threshold value. In one embodiment, the subject has not previously been administered CoQ10.

In some embodiments, the methods of the invention further comprise obtaining a biological sample from the subject.

In another aspect, the present invention provides method of treating cancer in a subject comprising: (a) obtaining a biological sample from the subject, (b) submitting the biological sample from the subject to obtain diagnostic information as to the level of PDIA3, (c) administering a therapeutically effective amount of CoQ10 to the subject if the level of PDIA3 in the biological sample is above a threshold level.

In still another aspect, the present invention provides methods of treating cancer in a subject, comprising: (a) obtaining diagnostic information as to the level of PDIA3 in a biological sample from the subject, and (b) administering CoQ10 to the subject if the level of PDIA3 in the biological sample is above a threshold level.

In yet another aspect, the present invention provides methods of treating cancer in a subject comprising: (a) obtaining a biological sample from the subject for use in identifying diagnostic information as to the level of PDIA3, (b) measuring the level of PDIA3 in the biological sample from the subject, (c) recommending to a healthcare provider to administer CoQ10 to the subject if the level of PDIA3 is above a threshold level.

In some embodiments the cancer to be treated is a solid tumor. In other embodiments, the cancer to be treated is selected from the group consisting of squamous cell carcinoma, glioblastoma, and pancreatic cancer.

In certain embodiments, the biological sample is selected from the group consisting of blood, serum, urine, organ tissue, biopsy tissue, feces, skin, hair, and cheek tissue.

In other embodiments, detecting the level of PDIA3 in a biological sample of the subject, comprises determining the amount of PDIA3 protein in the biological sample. In one embodiment, the level of PDIA3 protein is determined by immunoassay or ELISA. In another embodiment, the level of PDIA3 protein is determined by mass spectrometry.

In one embodiment, the level of PDIA3 is determined by (i) contacting the biological sample with a reagent that selectively binds to the PDIA3 to form a biomarker complex, and (ii) detecting the biomarker complex. In certain embodiments, the reagent is an anti-PDIA3 antibody that selectively binds to at least one epitope of PDIA3.

In other embodiments, the level of PDIA3 is determined by measuring the amount of PDIA3 mRNA in the biological sample. In certain embodiments, an amplification reaction is used for measuring the amount of PDIA3 mRNA in the biological sample. In one embodiment, the amplification reaction is (a) a polymerase chain reaction (PCR); (b) a nucleic acid sequence-based amplification assay (NASBA); (c) a transcription mediated amplification (TMA); (d) a ligase chain reaction (LCR); or (e) a strand displacement amplification (SDA).

In one embodiment, a hybridization assay is used for measuring the amount of PDIA3 mRNA in the biological sample. In certain embodiments, an oligonucleotide that is complementary to a portion of a PDIA3 mRNA is used in the hybridization assay to detect the PDIA3 mRNA.

In another aspect, the present invention provides kits for detecting PDIA3 in a biological sample from a subject having cancer and in need of treatment with CoQ10 comprising at least one reagent for measuring the level of PDIA3 in the biological sample from the subject, and a set of instructions for measuring the level of PDIA3 in the biological sample from the subject.

In one embodiment, the reagent is an anti-PDIA3 antibody. In another embodiment, the kit further comprising a means to detect the anti-PDIA3 antibody. In certain embodiments, the means to detect the anti-PDIA3 antibody is a detectable secondary antibody. In one embodiment, the reagent is an oligonucleotide that is complementary to a PDIA3 mRNA.

In one embodiment, the instructions set forth an immunoassay or ELISA for detecting the PDIA3 level in the biological sample. In another embodiment, the instructions set forth a mass spectrometry assay for detecting the PDIA3 level in the biological sample. In another embodiment, the instructions set forth an amplification reaction for assaying the level of PDIA3 mRNA in the biological sample.

In one embodiment, an amplification reaction is used for determining the amount of PDIA3 mRNA in the biological sample. In certain embodiments, the amplification reaction is a polymerase chain reaction (PCR); a nucleic acid sequence-based amplification assay (NASBA); a transcription mediated amplification (TMA); a ligase chain reaction (LCR); or a strand displacement amplification (SDA).

In one embodiment, the instructions set forth a hybridization assay for determining the amount of PDIA3 mRNA in the biological sample.

In another embodiment, the kit further comprises at least one oligonucleotide that is complementary to a portion of a PDIA3 mRNA.

In one embodiment, the instructions further set forth comparing the level of PDIA3 in the biological sample from the subject to a threshold value of PDIA3. In another embodiment, the instructions further set forth making a selection of the subject for treatment with CoQ10 based on the level of PDIA3 in the biological sample from the subject as compared to the threshold value of PDIA3.

### BRIEF DESCRIPTION OF FIGURES

The present disclosure is illustrated by way of example, and not limitation, in the figures of the accompanying drawings, in which like reference numerals indicate similar elements unless otherwise indicated.

FIG. 1 is a flowchart of a method for integrating molecular profile data and clinical records data for generating candidate biomarkers, in accordance with some embodiments.

FIG. 2 is a schematic network diagram depicting a system for implementation of methods described herein, in accordance with some embodiments.

FIG. 3 is a block diagram schematically depicting a system including modules for implementation of methods described herein, in accordance with some embodiments.

FIG. 4 is a flowchart of a method for analyzing data obtained from a clinical trial, in accordance with some embodiments.

FIG. 5 graphically depicts multiple annotated proteomics data files from multiple batches that are merged into a single data frame, in accordance with an embodiment.

FIG. 6 graphically depicts proteomics data files prior to filtering indicating which proteins are filtered where any protein that contains missing values for more than 60% of the samples is removed, in accordance with an embodiment.

FIG. 7A is a boxplot of proteomics expression data across samples prior to normalization.

FIG. 7B is a boxplot of the proteomics expression data of FIG. 7A after normalization according to the 60-less method, in accordance with an embodiment.

FIG. 8 graphically depicts a data set where missing data in the normalized proteomics data set is imputed, in accordance with an embodiment.

FIG. 9 graphically depicts a data set where missing data in a structural lipidomics data set is imputed, in accordance with an embodiment.

FIG. 10 includes four graphs illustrating the normalization process applied to the structural lipidomics data set including log2 raw values for a lipid class (top left), lipid values in the lipid class transformed by glog (top right), coefficient of variation of abundance (bottom left), and median centered glog transformed lipid values (bottom right), in accordance with an embodiment.

FIG. 11 graphically depicts a data set where missing data in the signaling lipidomics data set is imputed, in accordance with an embodiment.

FIG. 12 includes four graphs illustrating the normalization process applied to the signaling lipidomics data set including log2 raw values for a lipid class (top left), lipid values in the lipid class transformed by glog (top right), coefficient of variation of abundance(bottom left), and median centered glog transformed lipid values (bottom right), in accordance with an embodiment.

FIG. 13 graphically depicts annotated data files from multiple urine proteomics batches that are merged into a single data frame, in accordance with an embodiment.

FIG. 14 graphically depicts a urine proteomics data set prior to filtering indicating which proteins are filtered where any protein that contains missing values for more than 75% of the samples is removed, in accordance with an embodiment.

FIG. 15A shows urine proteomics data before normalization, in accordance with an embodiment.

FIG. 15B shows urine proteomics data after normalization by an approach that reduces the variance due to differences in hydration, in accordance with an embodiment.

FIG. 16 graphically depicts a data set where missing data in the normalized urine proteomics data set is imputed, in accordance with an embodiment.

FIG. 17 graphically depicts a metabolomics data set prior to filtering indicating which metabolite values are filtered where any metabolite that contains missing values for more than 60% samples is removed, in accordance with an embodiment.

FIG. 18 graphically depicts metabolomics data where missing data in the metabolomics data set is imputed, in accordance with an embodiment.

FIG. 19A is a graph of metabolomics data across samples prior to normalization.

FIG. 19B is a graph of metabolomics data across samples after normalization according to the 60-less method, in accordance with an embodiment.

FIG. 20 graphically depicts shows annotated metabolite data files from multiple batches and data sources that are merged into a single data frame, in accordance with an embodiment.

FIG. 21 is a graph of the frequency of log mean absolute deviation (MAD) values for lipidomics data (top) and a graph of percentiles of log(MAD) values for various lipids with a line showing the 45^{th} percentile cutoff where lipids with variability below the cutoff are considered invariant lipids and are removed (bottom), in accordance with an embodiment.

FIG. 22 graphically depicts a Bayesian network formed of an ensemble of Bayesian networks representing a complete (unsliced) data set where an edge frequency filter of 20% was applied to the ensemble prior to visualization, in accordance with an embodiment.

FIG. 23 graphically depicts a sub-network of the Bayesian network of FIG. 22 showing first first-degree neighbors of an exemplary outcome driver (potential biomarker) determined from analysis of network topography in accordance with an embodiment.

FIG. 24 graphically depicts a second sub-network of the Bayesian network of FIG. 22 showing first first-degree neighbors of a second exemplary outcome driver (potential biomarker) determined from analysis of network topography in accordance with an embodiment.

FIG. 25 graphically depicts a Bayesian network formed of an ensemble of Bayesian networks generated from a sliced data set including data collected from patients while they were experiencing severe adverse events related to blood and lymphatic system disorders where an edge frequency filter of 40% was applied to the ensemble prior to visualization, in accordance an embodiment.

FIG. 26 graphically depicts a Bayesian network formed of an ensemble of Bayesian networks generated from a sliced data set including data collected from patients while they were not experiencing severe adverse events related to blood and lymphatic system disorders where an edge frequency filter of 40% was applied to the ensemble prior to visualization, in accordance an embodiment.

FIG. 27 graphically depicts a differential (delta) network created from the pair of networks arising from the presence (FIG. 25) or absence (FIG. 26) of severe adverse events related to blood and lymphatic systems disorders, in accordance an embodiment.

FIG. 28 shows an exemplary patient dashboard for an example patient, in accordance with an embodiment. Clockwise from top left: Patient age, gender, race, site of initial tumor, treatment arm assigned, length of time on trial, last treatment cycle and tumor response, and disposition event; A subset of previous treatments that this patient has undertaken; Creatine levels, Prothombin time, and ECOG performance; Grade 3 adverse events experienced during the trial; Grade 2 adverse events experienced during the trial; Grade 1 adverse events experienced during the trial; Prothrombin time and Blood urea nitrogen levels during trial enrollment; Glucose, Hematocrit, Aspartate aminotransferase, alanine aminotransferase levels during trial enrollment; CoQ10 plasma concentration measured during trial enrollment; Geometric Mean of tumor measurements during trial enrollment, colored by tumor response (RECIST). In all figures, infusion of CoQ10 is indicated by gray shading. The beginning of cycle 2 is indicated by the vertical hashed line.

FIG. 29 shows an exemplary sample map (e.g., implemented as a web page) that visualizes available omic data for all patient samples in the CoQ10 clinical trial, in accordance with an embodiment.

FIG. 30 shows an exemplary interactive patient map (e.g., implemented as a web page) that provides an interactive visualization of tumor size measurements made for all patients enrolled in the trial in which tumor size is plotted as a percentage relative to initial tumor size, in accordance with an embodiment.

FIG. 31 shows a boxplot illustrating companion diagnostic biomarkers (CDx markers) measured prior to therapy that predict patient response, in accordance with an embodiment.

FIG. 32 shows a boxplot illustrating CDx markers measured prior to therapy predict severe adverse events, in accordance with an embodiment.

FIG. 33 graphically depicts portions of Bayesian networks including key drivers influencing patient response, in accordance with an embodiment.

FIG. 34 graphically depicts portions of Bayesian networks including key drivers influencing adverse events, in accordance with an embodiment.

FIG. 35 shows a boxplot illustrating candidate CDx markers measured prior to start of treatment to predict severe adverse events including the top 10 markers by differential expression, in accordance with an embodiment.

FIG. 36 schematically depicts a summary of the treatment groups in a Coenzyme Q10 (CoQ10) Phase I clinical trial related to treatment of solid tumors in Example 1. The trial contains a Coenzyme Q10 monotherapy (Mono) arm and a combination therapy arm in which Coenzyme Q10 is administered with the standard chemotherapeutic agents gemcitabine (GEM), 5-fluorouracil (5-FU), and docetaxel (DOC) to determine the maximum tolerated dose (MTD).

FIG. 37 shows FDG-PET scans before and 2, 10, 19 and 29 weeks after Coenzyme Q10 monotherapy in a patient with metastatic appendiceal cancer with surgery and heavily pretreated with multiple FOLFIRI and FOLFOX regimens in combination with irinotecan and Avastin, respectively in Example 1. Coenzyme Q10 monotherapy was initiated at 66 mg/kg dose and moved to 88 mg/kg dose at 22 weeks.

FIG. 38 schematically depicts an overview of the schedule for sampling and FDG PET-scans in patients enrolled in a Coenzyme Q10 (CoQ10) Phase I clinical trial related to treatment of solid tumors in Example 1.

FIG. 39A shows the mean concentration of Coenzyme Q10 in plasma of patients treated with Coenzyme Q10 monotherapy at 274 mg/kg/week or 342 mg/kg/week in Example 1.

FIG. 39B shows the mean concentration of Coenzyme Q10 in plasma of patients treated with Coenzyme Q10 in combination with standard chemotherapy. The dose of Coenzyme Q10 was 220 mg/kg/week or 274 mg/kg week in Example 1.

FIG. 39C shows a comparison of the data in FIG. 39A and 39B.

FIG. 40A shows a summary of demographic information and trial outcome for a patient enrolled in a Coenzyme Q10 Phase I clinical trial related to treatment of solid tumors in Example 1.

FIG. 40B shows tumor size progression for the patient relative to time of enrollment in Example 1.

FIG. 40C shows lab measurements for the patient for blood glucose (GLUC); hematocrit (HCT); aspartate transaminase (AST); and alanine transaminase (ALT) ratio in Example 1.

FIG. 40D shows the Adverse Events exhibited by the patient while enrolled on the clinical trial in Example 1 .

FIG. 40E shows FDG-PET scans of the patient before and after treatment with Coenzyme Q10.

FIG. 41 schematically depicts an overview of the data analytics process for identifying candidate biomarkers in Example 1.

FIG. 42A is an overview of results from the process of FIG. 41 including a boxplot showing the top ten differentially expressed molecules in blood measured before initial Coenzyme Q10 treatment that may potentially predict the efficacy of Coenzyme Q10 treatment for Example 1. Patients were stratified into overall clinical benefit and no clinical benefit groups for the analysis.

FIG. 42B shows bionetworks for the candidate biomarker protein disulfide-isomerase A3 (PDIA3) for Example 1.

FIG. 43 graphically depicts a Bayesian causal relationship network generated from data from all patients and schematically depicts a portion of the network related to the variable tumor size in Example 1.

FIG. 44 schematically depicts segmentation of time zero molecular profile data for responsive (overall clinical benefit) and refractory (no clinical benefit) patients in Example 1.

FIG. 45 schematically depicts analysis of time zero molecular profile data for responsive (overall clinical benefit) and refractory (no clinical benefit) patients to identify differently expressed molecules in Example 1.

FIG. 46 is a graph of the expression of time zero variables identified as predictive of patient response in Example 1.

FIG. 47 shows drivers of tumor response (RSORRES) harvested from the Bayesian network learned from the full data set in Example 2.

FIG. 48 shows insights into the mechanisms of action of CoQ10 harvested from the Bayesian network learned from the Cycle 1 patient data with 96 hour infusion schedule in Example 2.

FIG. 49 is a block diagram of a computing device that may be used to implement some embodiments of systems and methods described herein.

### DETAILED DESCRIPTION OF EXEMPLARY EMBODIMENTS

Some methods described herein enable efficient integration of a broad range of medical data including efficacy of treatment for a particular drug, medical history of the patient, and molecular profile data for the patient before, during and after treatment to identify novel relationships among these factors. For example, by using omics technology to analyze samples obtained from a patient, it is possible to perform a broad scale analysis of protein, lipid and metabolite levels throughout the course of treatment. In some embodiments, the omics data is combined with other clinical data such as demographic information, medical history, measurements of treatment efficacy, and pharmacokinetics of an administered drug to identify potential biomarkers that are indicative of patient response to the drug. These potential biomarkers could be used for a range of different applications, including selecting patients who are likely to be effectively treated by a drug, or who are likely to experience adverse events in response to the drug.

Embodiments described herein include methods, systems and computer-readable media for identifying one or more potential biomarkers for a clinical outcome related to administration of an agent and for patient stratification, e.g., in a subsequent clinical trial or for selecting patients for clinical treatment. Some embodiments provide methods and systems for processing and integrating clinical records data and molecular profile data from measurements of samples taken before, during, and/or after administration of an agent to a plurality of subjects, and analysis of the integrated data to identify one or more potential biomarkers for a clinical outcome related to administration of the agent (e.g., agent efficacy, an adverse event related to the agent). In some embodiments, the analysis includes generation of relationship networks (e.g., causal relationship networks, Bayesian networks, or Bayesian causal relationship networks) from slices of the integrated data and analysis of topological features of the causal relationship networks. In some embodiments, an in silico computational diagnostic patient map for determination of a subject response is generated from analysis of topological features of a causal relationship network. In some embodiments, the identified potential biomarkers for a clinical outcome related to administration the agent are used to predict a patient response to administration of the agent. In some embodiments, the agent is administered to subjects as part of a clinical trial. The potential biomarkers and analysis of the sliced merged molecular profile data and clinical records data can provide information for patient stratification, e.g., in a subsequent clinical trial or for selecting patients for clinical treatment.

The following description is presented to enable any person skilled in the art to make and use methods and system described herein. Various modifications to embodiments will be readily apparent to those skilled in the art, and the generic principles defined herein may be applied to other embodiments and applications without departing from the spirit and scope of the invention. Moreover, in the following description, numerous details are set forth for the purpose of explanation. However, one of ordinary skill in the art will realize that the invention may be practiced without the use of these specific details. Thus, the present disclosure is not intended to be limited to the embodiments shown, but is to be accorded the widest scope consistent with the principles and features disclosed herein.

Definitions

As used herein, certain terms intended to be specifically defined, but are not already defined in other sections of the specification, are defined herein.

As used herein, the term “slicing a merged data set” refers to selecting one or more subsets of the merged data set using one or more criteria. As used herein, the terms “sliced data set” or “slices data sets” refer to data set(s) that are subsets of the merged data set resulting from the slicing operation and are also referred to a selected data set(s) herein.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited to.”

The term “or” is used herein to mean, and is used interchangeably with, the term “and/or,” unless context clearly indicates otherwise.

The term “such as” is used herein to mean, and is used interchangeably, with the phrase “such as but not limited to.”

The term “microarray” refers to an array of distinct polynucleotides, oligonucleotides, polypeptides (e.g., antibodies) or peptides synthesized on a substrate, such as paper, nylon or other type of membrane, filter, chip, glass slide, or any other suitable solid support.

The terms “disorders” and “diseases” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

As used herein, “cancer” refers to all types of cancer or neoplasm or malignant tumors found in humans, including, but not limited to: leukemias, lymphomas, melanomas, carcinomas and sarcomas. As used herein, the terms or language “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also cancer stem cells, as well as cancer progenitor cells or any cell derived from a cancer cell ancestor. This includes metastasized cancer cells, and in vitro cultures and cell lines derived from cancer cells. A “solid tumor” is a tumor that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. The tumor does not need to have measurable dimensions.

The term “expression” includes the process by which a polypeptide is produced from polynucleotides, such as DNA. The process may involves the transcription of a gene into mRNA and the translation of this mRNA into a polypeptide. Depending on the context in which it is used, “expression” may refer to the production of RNA, protein or both.

The terms “level of expression of a gene” or “gene expression level” refer to the level of mRNA, as well as pre-mRNA nascent transcript(s), transcript processing intermediates, mature mRNA(s) and degradation products, or the level of protein, encoded by the gene in the cell.

The term “genome” refers to the entirety of a biological entity's (cell, tissue, organ, system, organism) genetic information. It is encoded either in DNA or RNA (in certain viruses, for example). The genome includes both the genes and the non-coding sequences of the DNA.

The term “proteome” refers to the entire set of proteins expressed by a genome, a cell, a tissue, or an organism at a given time. More specifically, it may refer to the entire set of expressed proteins in a given type of cells or an organism at a given time under defined conditions. Proteome may include protein variants due to, for example, alternative splicing of genes and/or post-translational modifications (such as glycosylation or phosphorylation).

The term “transcriptome” refers to the entire set of transcribed RNA molecules, including mRNA, rRNA, tRNA, and other non-coding RNA produced in one or a population of cells at a given time. The term can be applied to the total set of transcripts in a given organism, or to the specific subset of transcripts present in a particular cell type. Unlike the genome, which is roughly fixed for a given cell line (excluding mutations), the transcriptome can vary with external environmental conditions. Because it includes all mRNA transcripts in the cell, the transcriptome reflects the genes that are being actively expressed at any given time, with the exception of mRNA degradation phenomena such as transcriptional attenuation.

The study of transcriptomics, also referred to as expression profiling, examines the expression level of mRNAs in a given cell population, often using high-throughput techniques based on DNA microarray technology.

The term “metabolome” refers to the complete set of small-molecule metabolites (such as metabolic intermediates, hormones and other signaling molecules, and secondary metabolites) to be found within a biological sample at a given time under a given condition. The metabolome is dynamic, and may change from second to second.

The term “lipidome” refers to the complete set of lipids to be found within a biological sample at a given time under a given condition. The lipidome is dynamic, and may change from second to second.

As used herein, and agent refers to something administered to subjects. The term agent includes, but is not limited to, a treatment or a potential treatment for a disease or a disorder, and a potential or known pharmaceutical agents for treatment of a disease or disorder.

Other terms not explicitly defined in the instant application have meaning as would have been understood by one of ordinary skill in the art.

Although the description below is presented in some portions as discrete steps, it is for illustration purpose and simplicity, and thus, in reality, it does not imply such a rigid order and/or demarcation of steps. Moreover, the steps of the invention may be performed separately, and the invention provided herein is intended to encompass each of the individual steps separately, as well as combinations of one or more (e.g., any one, two, three, four, five, six or all seven steps) steps, which may be carried out independently of the remaining steps.

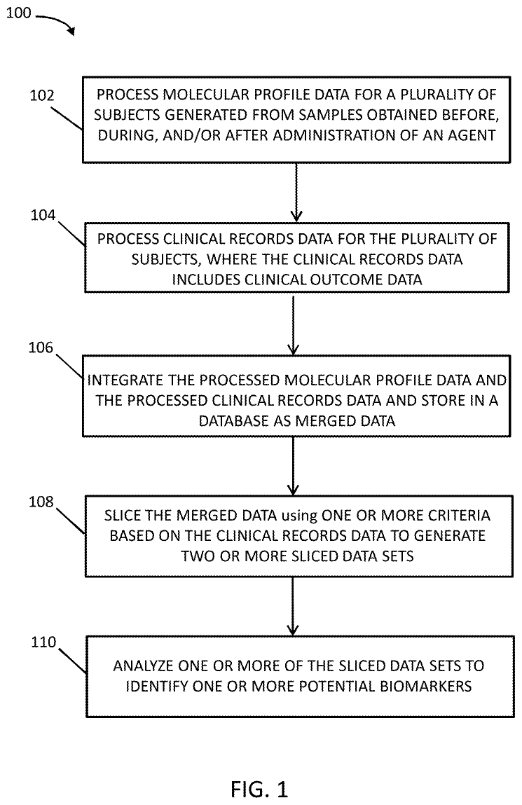

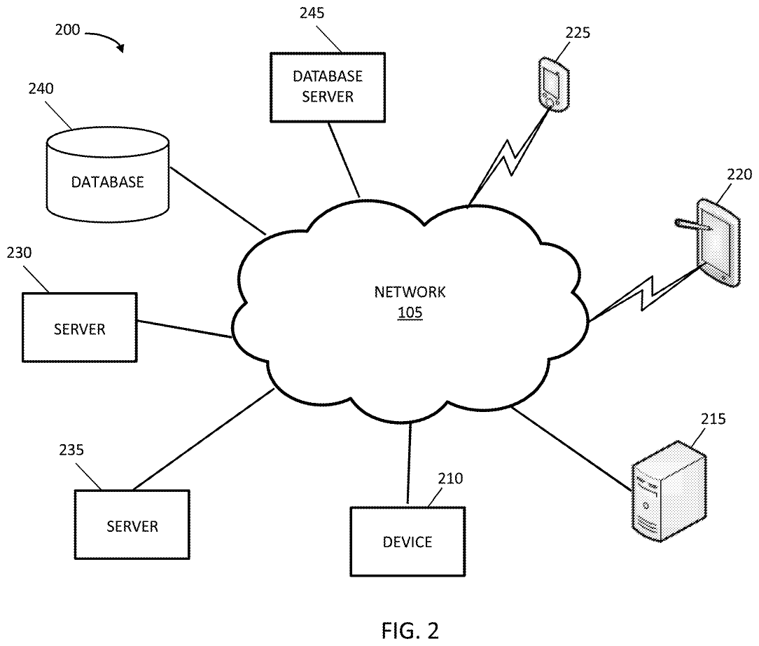

FIG. 1 illustrates an example flow diagram of a method 100 for integrating molecular profile data and clinical records data for generating potential biomarkers for a clinical outcome related to administration of an agent, according to an example embodiment. The method is a computer-implemented method. An example system for implementing method 100 is described below with respect to FIGS. 2, 3 and 49; however, one of ordinary skill in the art will appreciate that one or more other systems may be used to implement the method.

At step 102, molecular profile data for each subject in a plurality of subjects is processed. In some embodiments, the molecular profile data for each subject includes one or more of proteomics, metabolomics, lipidomics, genomics, transcriptomics, microarray and sequencing data generated from analysis of a plurality of samples obtained from the subjects. In some embodiments, the molecular profile data for each subject includes two or more of proteomics, metabolomics, lipidomics, genomics, transcriptomics, microarray and sequencing data generated from analysis of a plurality of samples obtained from the subjects. In some embodiments, the molecular profile data for each subject includes three or more of proteomics, metabolomics, lipidomics, genomics, transcriptomics, microarray and sequencing data generated from analysis of a plurality of samples obtained from the subjects.

For each subject, the plurality of samples includes samples obtained before, during, and/or after administration of the agent to the subject. For example, in some embodiments the plurality of samples includes samples obtained before and during administration of the agent to the subject. In some embodiments, the plurality of samples includes samples obtained during and after administration of the agent to the subject. In some embodiments, the plurality of samples includes samples obtained before and after administration of the agent to the subject. In some embodiments, the plurality of samples includes samples obtained before, during, and after administration of the agent to the subject.

In some embodiments, the agent is being evaluated as a potential treatment for a disease or a disorder. In some embodiments, the agent is administered to the plurality of subjects as part of a clinical trial. In some embodiments, the agent is administered to the plurality of subjects as part of a phase I clinical trial. In some embodiments the method includes administering the agent to the plurality of subjects.

In some embodiments, the samples from each subject include one or more of blood, tissue, urine, secretion, sweat, sputum, stool, and mucous samples, and cultures thereof. In some embodiments, the samples from each subject include comprise two or more of blood, tissue, urine, secretion, sweat, sputum, stool, and mucous samples, and cultures thereof. In some embodiments, the blood sample is selected from the group consisting of whole blood, serum, plasma and buffy coat. In some embodiments, the tissue is obtained through biopsy. In certain embodiments, the tissue is a tumor tissue.

In some embodiments, the method further includes, for each subject, analyzing the plurality of samples obtained from subject to obtain the molecular profile data. Further description of methods to obtain the molecular profile data appears in the section below entitled “Generation of Molecular Profile Data.”

In some embodiments, processing the molecular profile data includes one or more of combining data collected at different time points over the course of the treatment for the plurality of subjects, filtering to remove infrequently measured variables, normalizing the data by removing systematic biases to ensure samples are comparable across different batches employed during measurement of the data, and imputing any variable not measured for a particular subject of the plurality of subjects. Additional description of processing of molecular profile data appears below in the section entitled “Omics Data Processing.”

At step 104, clinical records data, also referred to as “clinical data” herein, for the plurality of subjects is processed. The clinical records data for each subject includes data based on samples obtained from the subject and/or measurements made of the subject before, during, and/or after administration of the agent. For example, in some embodiments, the clinical records data includes data based on samples obtained before and during administration of the agent to the subject. In some embodiments, the clinical records data includes data based on samples obtained during and after administration of the agent to the subject. In some embodiments, the clinical records data includes data based on samples obtained before and after administration of the agent to the subject. In some embodiments, the clinical records data includes data based on samples obtained before, during, and after administration of the agent to the subject. In some embodiments, the clinical records data includes data based on measurements made of the subject before and during administration of the agent to the subject. In some embodiments, the clinical records data includes data based on measurements made of the subject during and after administration of the agent to the subject. In some embodiments, the clinical records data includes data based on measurements made of the subject before and after administration of the agent to the subject. In some embodiments, the clinical records data includes data based on measurements made of the subject before, during, and after administration of the agent to the subject.

The clinical records data includes clinical measurements made on samples obtained from subjects and/or clinical measurements made on subjects relevant to assessment of general health status of subjects or status of a disease or disorder of interest. For example, clinical measurements for general health status assessments include some or all of weight, height, body mass index (BMI), glucose level, cholesterol level, blood pressure, and changes thereof. For example, clinical measurements for assessment of cancer status include some or all of tumor size, PET scan, FDE-PET scan, cancer biopsy, pharmacokinetics of a potential or known cancer therapeutic agent, levels of blood glucose (GLUC), hematocrit (HCT), aspartate transaminase (AST) and alanine transaminase (ALT), and changes thereof. In some embodiments, the clinical records data includes medical history data and/or demographic data of subjects. Demographic data includes, but is not limited to, any or all of age, gender and ethnicity. The clinical records data includes clinical outcome data. In some embodiments, the clinical outcome data includes data related to the efficacy of the agent for treatment of a disease or disorder. For example, the clinical outcome data can include data regarding a state or status of a disease or a disorder in the subject at a particular time before, during and/or after treatment. In some embodiments, the clinical outcome data includes data related to adverse events associated with administration of the agent. For example, the clinical outcome data can include information related to the occurrence of an adverse event during or after administration of the agent. In some embodiments, the agent is a treatment or a potential treatment for a disease or disorder and the clinical outcome data includes data indicating whether a subject exhibited an overall clinical benefit or no clinical benefit in response to treatment with the agent. In embodiments, clinical records data is retrieved or obtained from conventional medical history records or a mobile wearable device.

In some embodiments, the clinical records data also includes one or more of pharmacokinetics data, medical history data, laboratory test data, demographic data and data from a mobile wearable device.

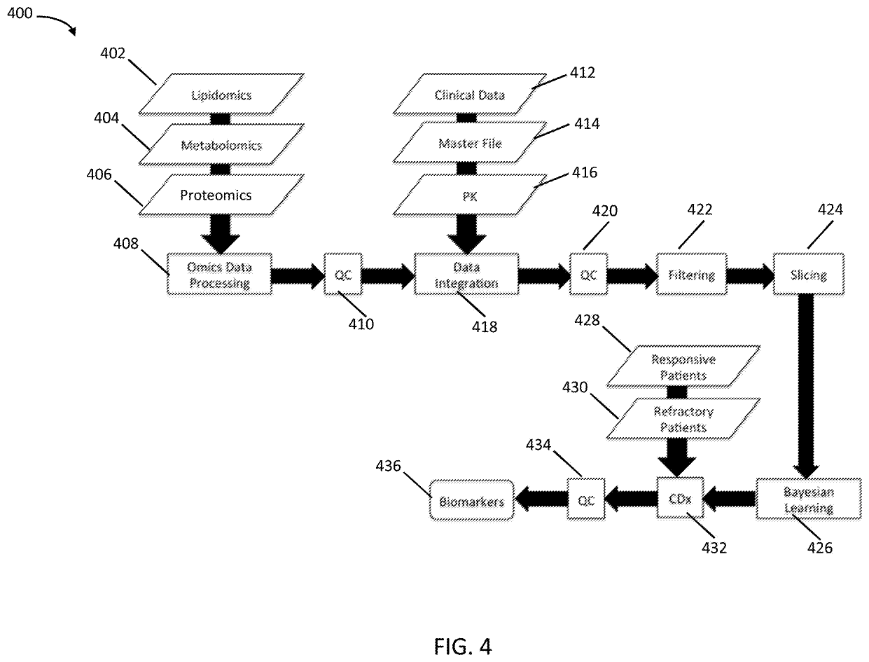

In some embodiments the clinical data is provided by clinical data monitors. Processing of the clinical data may enable efficient integration of the molecular profile data with the clinical records data. For example, the clinical data may be provided in multiple different formats (e.g., narrative, continuous, discrete, Boolean) that needs to be standardized for different subjects. Additional description of processing of clinical data appears below in the description of FIG. 4.

At step 106, the processed molecular profile data and the processed clinical records data are integrated, and stored in a database as merged data. In some embodiments, integration of the processed molecular profile data and the processed clinical records data includes reconciling duplicated clinical records data and resolving discrepancies. In some embodiments, integration of the processed molecular profile data and the processed clinical records data includes filtering the merged data to remove molecular data for which corresponding clinical records data is missing. In some embodiments, because data types are collected with different frequencies, all quantitative clinical records, such as tumor size, are matched to omics sample time points by interpolation (e.g., linear interpolation), as needed. In some embodiments, samples for pharmacokinetics (PK) and samples for molecular profile data are obtained at the same time points (e.g., on the same dates) for a particular subject, which aids integrating the clinical data and with the molecular profile data and avoids the need to determine interpolated PK values for time points corresponding to molecular profile sample collection.

Additional description of integration of the processed clinical data and the processed records data appears below in the description of FIG. 4.

At step 108, the merged data is sliced based on one or more criteria obtained from the clinical records data to generate two or more sliced data sets. As used herein, slicing refers to splitting the data into groups based on criteria or features. In some embodiments, the one or more criteria for slicing the merged data includes a phenotypic classification, such as age, gender, or ethnicity. In some embodiments, the one or more criteria for slicing the merged data includes clinical outcome data, such as apparent responsivity to the agent or occurrence of an adverse event. For example, in some embodiments the merged data is sliced based on a subject having experienced an adverse event to create two sliced data sets: one corresponding to data for subjects that experienced the adverse events and one corresponding to data for subjects that did not experience the adverse event. As another example, in some embodiments the data is sliced by criteria such as change in tumor size during treatment for a clinical trial for a cancer drug to create sliced data sets of subjects (e.g., patients) responsive to the agent (e.g., that exhibited an overall clinical benefit) and subject (e.g., patients) who were refractory (e.g., that exhibited no clinical benefit). In another embodiment, the merged data is sliced by subject to create a sliced data set for each individual subject (e.g., patient). In some embodiments, the data may be sliced by a demographic trait, such as age, gender or ethnicity. In some embodiments, the data may be sliced by criteria such as body mass index, presence of elevated glucose levels, presence of elevated blood pressure, certain events in the medical history, etc.

In some embodiments, the merged data is sliced multiple times based on different criteria. For example the merged data could be sliced in one slice that includes data for all subjects, and also sliced based on the clinical outcome data (e.g., into one slice including data from subjects that exhibited an overall clinical benefit in response to treatment with the agent and another slice including data from subjects that exhibited no clinical benefit in response to treatment with the agent).

At step 110, one or more of the sliced data sets are analyzed to identify one or more potential biomarkers for a clinical outcome related to administration of the agent. In some embodiments, the sliced data sets are analyzed using one or more of artificial intelligence methods (e.g., AI networks), statistical methods (e.g., differential expression), and machine learning methods to identify the potential biomarkers for the clinical outcome related to administration of the agent. In some embodiments, the sliced data sets are analyzed using two or more of artificial intelligence methods, statistical methods, and machine learning methods to identify the potential biomarkers for the clinical response related to administration of the agent. Examples of the use of artificial intelligence methods (e.g., generation of Bayesian causal relationship networks), statistical methods (e.g., statistical analysis of differentially expressed variables), and machine learning methods (e.g., regression analysis to select relatively uncorrelated potential biomarkers from sets of possible biomarkers produced from other techniques) to identify potential biomarkers for agent efficacy and adverse reactions are described below with respect to FIG. 4 and Examples 1 and 2.

In some embodiments, analyzing one or more of the sliced data sets to identify one or more potential biomarkers includes generation of one or more relationship networks (e.g., Bayesian causal relationship networks or Bayesian networks) based on one or more of the sliced data sets. A description of generation of Bayesian causal relationship networks is provided below in the section entitled “Generation of Bayesian Causal Relationship Networks using an AI-Based System.”

In embodiments employing the generation of one or more causal relationship networks, analysis of the generated one or more causal relationship networks identifies one or more nodes corresponding to one or more output drivers. In some embodiments, analysis of topological features of the causal relationship networks is used for identifying the one or more nodes corresponding to one or more output drivers In some embodiments, the identified one or more output drivers are the one or more potential biomarkers for the clinical outcome related to administration of the agent. In some embodiments, the output drivers are identified as possible biomarkers, and additional analysis is conducted to select the one or more potential biomarkers from a group of possible biomarkers. In such an embodiment, the one or more potential biomarkers are selected from a group of possible biomarkers that includes the one or more output drivers.

In some embodiments, analysis of the generated one or more causal relationship networks includes identifying as outcome drivers variables corresponding to nodes connected to a node corresponding to the clinical outcome in one or more of the generated causal relationship networks by relationship having a degree of connection of less than n. For example, if n is 1, outcome drivers are variables nodes directly connected to the outcome node by a relationship. As another example, if n is 2, outcome drivers are variables nodes connected to the outcome node by two relationships and an intervening node. In various embodiments, n is 1, 2, 3, 4, 5, 6, 7, 8, 9, or 10. In some embodiments, n is 3 or 2 or 1.

In some embodiments, the data is sliced by subject. In some embodiments, a first plurality of causal relationship networks is generated, each based on one of the first plurality of sliced data sets corresponding to subjects that exhibited the clinical outcome, and a second plurality of causal relationship networks is generated each based on one of the second plurality of sliced data sets corresponding to subjects that did not exhibit the clinical outcome. One or more first commonalities are identified among the first plurality of causal relationship networks and one or more second commonalities are identified among the second plurality of causal relationship networks. Comparison of the first commonalities and the second commonalities is used to identify the one or more outcome drivers.

In some embodiments, the merged data is sliced by clinical and the generated two or more sliced data sets include a first sliced data set including data corresponding to one or more subjects that exhibited the clinical outcome and a second sliced data set including data corresponding to one or more subjects that did not exhibit the clinical outcome. In some embodiments, a first causal relationship network is generated based on the first sliced data set corresponding to subjects that exhibited the clinical outcome, and a second causal relationship network is generated based on the second sliced data set corresponding to subjects that did not exhibit the clinical outcome. In some embodiments, the one or more outcome drivers are identified based on a comparison the first causal relationship corresponding to subjects that exhibited the clinical outcome and the second causal relationship corresponding to subjects that did not that did not exhibit the clinical outcome. In some embodiments, a differential (delta) network is generated based on the first causal relationship network and the second causal relationship network and the one or more outcome drivers are identified from the generated differential causal relationship network

In some embodiments, analyzing one or more of the sliced data sets to identify one or more potential biomarkers for a clinical outcome related to administration of the agent also includes identifying one or more variables differentially expressed between sliced data sets that were sliced based on a clinical outcome through a statistical analysis. In some embodiments, such a statistical analysis of differential expression employs a two-sample t-test or limma methodology. In some embodiments, such a statistical analysis of differentially expressed variables includes performing a regression analysis. In some embodiment, the statistical analysis produces a list of the variables showing the largest differential in expression between data sets sliced based on clinical outcome, which are identified as possible biomarkers from which subset of potential biomarkers are identified.

In some embodiments, many (e.g., tens to hundreds) of outcome drivers and many (e.g., tens to hundreds) differentially expressed variables may be identified as possible biomarkers; however, many of these possible biomarkers are likely strongly correlated with each other. For efficiency, it is advantageous to identify a set of biomarkers that are strongly predictive or correlated with the clinical outcome of interest, but are relatively uncorrelated with each other (e.g., orthogonal biomarkers) such that each additional biomarker provides additional information. In some embodiments, additional analysis is performed to determine one or more potential biomarkers that are relatively uncorrelated with each other (e.g., orthogonal) from the possible biomarkers identified.

In some embodiments, the outcome drivers identified from generated networks and the top differential expressed variables form a group of possible biomarkers and the one or more potential biomarkers are identified as a subset of the group of possible biomarkers using machine learning. For example, in some embodiments machine learning is used to analyze the identified outcome drivers and the one or more differentially expressed variables as possible biomarkers and, based on the analysis, selecting a subset of the possible biomarkers as the one or more potential biomarkers, wherein the machine learning penalizes possible biomarkers that are strongly correlated with other possible biomarkers and rewards possible biomarkers based on a level of correlation with the clinical outcome, thereby identifying one or more potential biomarkers for the clinical outcome. In some embodiments, the machine learning employed to analyze the possible biomarkers applies logistic regression with the elastic net penalty as described below in the section entitled “Determination of Potential Biomarkers (e.g., Companion Diagnostics CDx).”

In some embodiments, the one or more potential biomarkers are potential biomarkers for agent efficacy or for an adverse event. In some embodiments, the method 100 is a method for identifying one or more potential biomarkers for the occurrence of an adverse event related to administration of the agent.

When the agent is a potential treatment for a disease or a disorder, the method 100 may be a method for patient stratification to predict which patient would be responsive to treatment by the agent, to predict which patients would be likely have adverse events when treated with the agent, or both. In some embodiments, the method further includes employing the identified one or more potential biomarkers for patient stratification, e.g., in a subsequent clinical trial or for selecting patients for clinical treatment. In some embodiments, the potential biomarkers can be used for patient stratification to determine which patients are enrolled in the subsequent clinical trial. In some embodiments, the potential biomarkers can be used for patient stratification to determine the patients that receive the agent in the subsequent clinical trial.

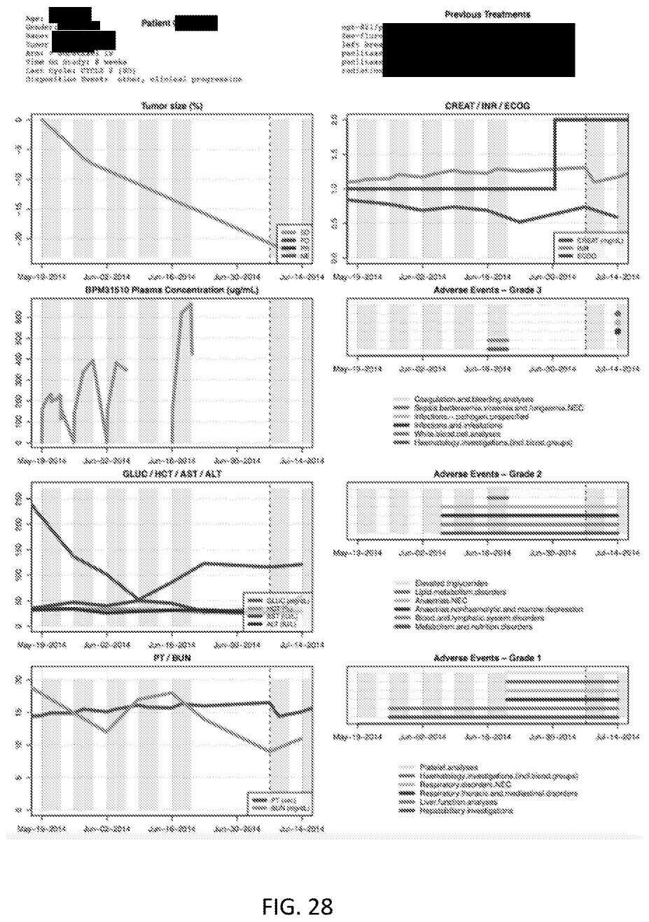

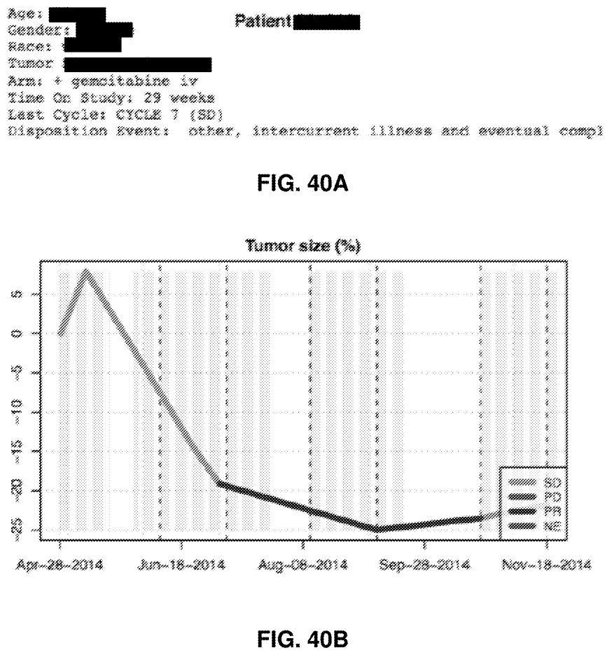

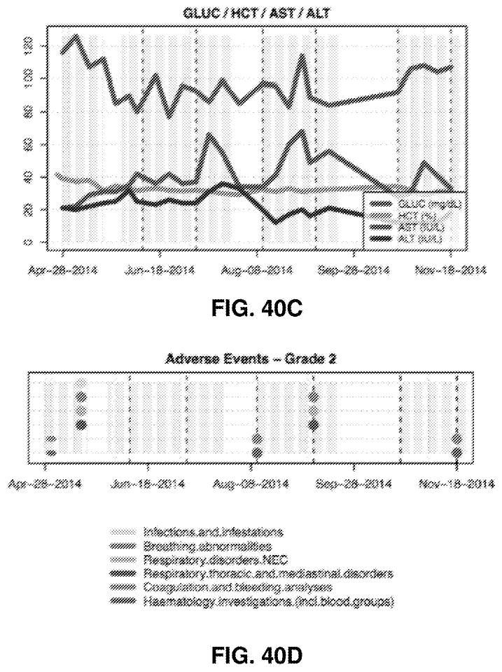

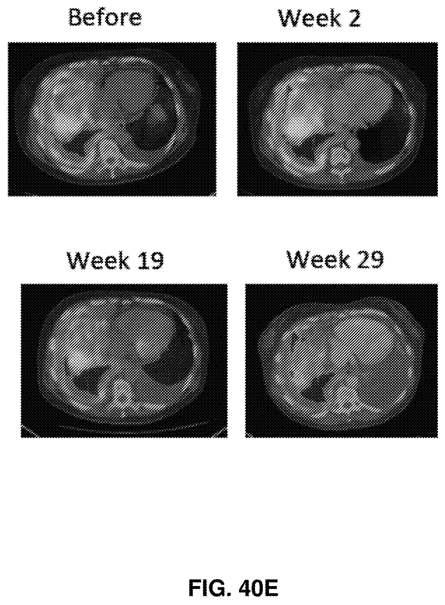

In some embodiment, the method 100 also includes displaying a subject-specific profile on a display device. The subject-specific profile comprises a graphical representation of clinical records data. The subject-specific profile comprises a graphical representation of demographic information for the subject and a graphical representation of outcome information for the subject. The graphical representation of outcome information for the subject may comprise a graphical representation of adverse event information for the subject, and a graphical representation of information regarding responsivity to the agent. A subject-specific profile in the form of a patient profile is shown and described with respect to FIG. 28 and another patient file is described below with respect to Example 1 and shown in FIGS. 40A-40D.

Some embodiments include a method of generating an in silico computational diagnostic patient map for determination of a subject response from analysis of topological features of a causal relationship network (e.g., a Bayesian causal relationship network) generated from a sliced merged data set of processed molecular profile data and processed clinical records performed according to method 100 described above.

In some embodiments, an in vitro cell model of a disease or disorder may be established and Bayesian causal relationship networks generated to identify molecular hubs related to a disease or disorder, or potential modulators of a disease or disorder. Details regarding methods and systems for identifying modulators of a disease or disorder using Bayesian causal relationship networks based on in vitro cells models appear in U.S. Patent Application Publication No. US2012/0258874A1, entitled, “Interrogatory Cell-Based Assays and Uses Therof,” the entire contents of which is incorporated by reference herein. In some embodiments, the potential modulators of a disease or disorder identified using the in vitro cell models can be compared with the potential biomarkers identified from analysis of the sliced data to obtain information regarding a mechanism of action for the potential biomarkers. The in vitro cell model may be analyzed using the Berg Interrogative Biology™ Informatics Suite, which is a tool for understanding a wide variety of biological processes, such as disease pathophysiology, and the key molecular drivers underlying such biological processes, including factors that enable a disease process. Some exemplary embodiments employ the Berg Interrogative Biology™ Informatics Suite to gain novel insights into disease interactions with respect to other diseases, medical drugs, biological processes, and the like. Some exemplary embodiments include systems that may incorporate at least a portion of, or all of, the Berg Interrogative Biology™ Informatics Suite.

FIG. 2 illustrates a network diagram depicting an example system 200 that can be used in part or in full in to implement methods described herein in accordance with an embodiment. The system 200 can include a network 205, a device 210, a device 215, a device 220, a device 225, a server 230, a server 235, a database(s) 240, and a database server(s) 245. Each of the devices 210, 215, 220, 225, servers 230, 235, database(s) 240, and database server(s) 245 is in communication with the network 205.

In an embodiment, one or more portions of network 205 may be an ad hoc network, an intranet, an extranet, a virtual private network (VPN), a local area network (LAN), a wireless LAN (WLAN), a wide area network (WAN), a wireless wide area network (WWAN), a metropolitan area network (MAN), a portion of the Internet, a portion of the Public Switched Telephone Network (PSTN), a cellular telephone network, a wireless network, a WiFi network, a WiMax network, any other type of network, or a combination of two or more such networks.

The devices 210, 215, 220, 225 may include, but are not limited to, work stations, personal computers, general purpose computers, Internet appliances, laptops, desktops, multi-processor systems, set-top boxes, network PCs, wireless devices, portable devices, wearable computers, cellular or mobile phones, portable digital assistants (PDAs), smartphones, tablets, ultrabooks, netbooks, multi-processor systems, microprocessor-based or programmable consumer electronics, mini-computers, and the like. Each of the devices 210, 215, 220, 225 may connect to network 205 via a wired or wireless connection.

In some embodiments, server 230 and server 235 may be part of a distributed computing environment, where some of the tasks/functionalities are distributed between servers 230 and 235. In some embodiments, server 230 and server 235 are part of a parallel computing environment, where server 230 and server 235 perform tasks/functionalities in parallel to provide the computational and processing resources necessary to generate the Bayesian causal relationship networks described herein.

In some embodiments, each of the server 230, 235, database(s) 240, and database server(s) 245 is connected to the network 205 via a wired connection. Alternatively, one or more of the server 230, 235, database(s) 240, or database server(s) 245 may be connected to the network 205 via a wireless connection. Although not shown, database server(s) 245 can be directly connected to database(s) 240, or servers 230, 235 can be directly connected to the database server(s) 245 and/or database(s) 240. Server 230, 235 comprises one or more computers or processors configured to communicate with devices 210, 215, 220, 225 via network 205. Server 230, 235 hosts one or more applications or websites accessed by devices 210, 215, 220, and 225 and/or facilitates access to the content of database(s) 240. Database server(s) 245 comprises one or more computers or processors configured to facilitate access to the content of database(s) 240. Database(s) 240 comprise one or more storage devices for storing data and/or instructions for use by server 230, 235, database server(s) 245, and/or devices 210, 215, 220, 225. Database(s) 240, servers 230, 235, and/or database server(s) 245 may be located at one or more geographically distributed locations from each other or from devices 210, 215, 220, 225. Alternatively, database(s) 240 may be included within server 230 or 235, or database server(s) 245.

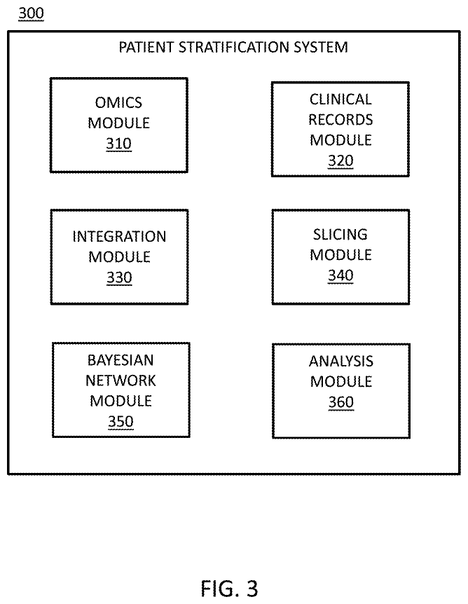

FIG. 3 is a block diagram showing a system 300 implemented in modules according to an example embodiment. In some embodiments, the modules include an omics module 310, a clinical records module 320, an integration module 330, a slicing module 340, a Bayesian network module 350, and an analysis module 360. In an example embodiment, one or more of modules 310, 320, 330, 340, 350 and 360 are included in server 230 and/or server 235 while other of the modules 310, 320, 330, 340, 350, and 360 are provided in the devices 210, 215, 220, 225.

In alternative embodiments, the modules may be implemented in any of devices 210, 215, 220, 225. The modules may comprise one or more software components, programs, applications, apps or other units of code base or instructions configured to be executed by one or more processors included in devices 210, 215, 220, 225.

Although modules 310, 320, 330, 340, 350, 360 are shown as distinct modules in FIG. 3, it should be understood that modules 310, 320, 330, 340, 350, and 360 may be implemented as fewer or more modules than illustrated. It should be understood that any of modules 310, 320, 330, 340, 350, and 360 may communicate with one or more external components such as databases, servers, database server, or other devices.

In some embodiments, the omics module 310 is a hardware-implemented module configured to receive and manage molecular profile data obtained from analysis of samples from the plurality of subjects. The omics module 310 may be configured to receive any of proteomics, metabolomics, lipidomics, genomics, transcripomics, microarray and sequencing data regarding the sample. In some embodiments, the omics module 310 is configured to receive the omics data from systems used to generate the omics data. The omics module 310 is also configured to process the molecular profile data to produced processed molecular profile data. In some embodiments, the omics module 310 is configured to combine data collected at different time points over the course of the treatment for the plurality of subjects. In some embodiments, the omics module 310 is configured to filter the data to remove infrequently measured variables. In some embodiments, the omics module 310 is configured to normalize the data by removing systematic biases to ensure samples are comparable across different batches employed during analysis of the samples to generate the data. In some embodiments, the omics module 310 is configured to impute any variable not measured for a particular subject of the plurality of subjects. In some embodiments, the omics module 310 is configured to combine data, filter data, normalize data and impute variables not measured.

In some embodiments, the clinical records module 320 is a hardware-implemented module configured to receive and manage clinical records data for the plurality of subjects. The clinical records module 320 is also configured to process the clinical records data.

In some embodiments, the integration module 330 is a hardware-implemented module configured to integrate the processed molecular profile data and the processed clinical records data for the plurality of subjects and store integrated data in a database as merged data.

In some embodiments, the slicing module 340 is hardware-implemented module configured to slice the merged data based on criteria obtained from the clinical records to generate two or more sliced data sets.

Some embodiments include a Bayesian network generation module 350 that may be a hardware-implemented module configured to generate Bayesian causal relationship networks from one or more of the sliced data sets. In some embodiments, the Bayesian network module 350 is also configured to identify outcome drivers from the generated Bayesian causal relationship networks.

The analysis module 360 may be a hardware-implemented module configured to identify biomarkers for prediction of a clinical outcome related to administration of an agent. In some embodiments, analysis of the generated Bayesian networks to identify the outcome drivers may be conducted by the analysis module 360 instead of the Bayesian network module 350, or in conjunction with the Bayesian network model. In some embodiments, the analysis module 360 may be configured to conduct statistical analysis for identification of differentially expressed variables. In some embodiments, the analysis module 360 may also be configured to manage and apply machine learning algorithms to possible biomarkers to identify potential biomarkers (predictors) for prediction of a clinical outcome related to administration of the agent. The analysis module 360 may also be configured to apply the identified potential biomarkers (predictors) to a subsequent clinical trial of the agent. In some embodiments, the analysis module 360 may include multiple different modules that perform different aspects of the analysis (e.g., an outcome driver identification module, a differential expression module and machine learning module).

FIG. 4 illustrates an example flow diagram for the clinical trial analytics workflow (CTAW) 400 for analyzing data obtained from a clinical trial, according to an embodiment. Although method 400 is described in the context of a clinical trial, one skilled in the art will appreciate that the method may be applied outside the context of a clinical trial in some other trial, experiment, or study in which an agent is administered to a plurality of subjects. Samples are collected from a plurality of subjects during the clinical trial before, during and/or after administration of an agent to the plurality of subjects. In an example embodiment, samples (e.g., blood, tissue, urine samples) are obtained from subjects (e.g., patients) and interrogated by omics profiling to produce lipidomics data 402, metabolomics data 404, and proteomics data 406. Further details on processing collected samples to produce lipidomics data 402, metabolomics data 404 and proteomics data 406 are provided below in the section entitled “Generation of Molecular Profile Data.” In some embodiments, additional data such as genomic data and transcriptomics data is also generated from analysis of the samples.

At step 408, omics data processing occurs taking the lipidomics data 402, metabolomics data 404 and proteomics data 406 as inputs. In embodiments including genomics data and/or transcriptomics data, this data is also included in omics data processing. Technology-specific pipelines convert these raw omics measurements into processed molecular profile data by merging to combine data collected at different times during the clinical trial. In some embodiments, this processing includes filtering to remove variables that are measured infrequently. The data is further normalized by removing systematic biases to ensure samples are comparable across batches, as needed. In some embodiments, imputation is used to infer the level of any variable that was not measured in a particular sample, as needed. Further details regarding the omics processing is included below under the section entitled “Omics Data Processing.”.

At step 410, in some embodiments, data processing reliability of the omics data processing is ensured by quality control steps including testing if raw data files follow expected formatting, and making intuitive visualizations that track each step of the omics data processing. To ensure traceability, all outputs from the quality control are written to a central log file (for example, by the omics module 310) in some embodiments.

Clinical data 412 is obtained. Additional information regarding the input of the clinical data is provided below in the section entitled “Clinical Records Data.” In some embodiments, a master file 414 is created or obtained that identifies which samples used for molecular profiling correspond to which patient and the point in time that the sample was taken. The point in time may be recorded relative to relevant starting time point for the particular subject (e.g., time 0 may correspond to the beginning of a treatment cycle). In some embodiments, pharmacokinetic data is also obtained 416. Pharmacokinetic data 416 is considered a type of clinical records data herein and in some embodiments, the pharmacokinetic data 416 is provided along with the clinical data 412. Additional information regarding the input of the clinical data and generation of the master file is provided below in the section entitled “Clinical Records Data.”

At step 418, the processed molecular profile data is integrated with the clinical data. In some embodiments, the processed molecular profile data (e.g., omics data) is merged with clinical records by means of the Master File 414, which specifies the subject (e.g., by a patient ID) and a time point corresponding to each sample collected. Clinical data 412 in the form of clinical records provided by clinical data monitors, which can include pharmacokinetic data 416, is then merged with the processed molecular profile data, and the merged data is stored in a database. Given the patient ID and time of collection, available clinical records may be matched in time to omics data to generate an integrated data set containing omics data and clinical records. The resulting merged data in the database can include any or all of demographics, treatments, disease status or disorder status, clinical outcome data (e.g., such as tumor size measurements in clinical trials for cancer treatments, adverse events, etc.), lab measurements, pharmacokinetics data, proteomics, lipidomics, and metabolomics collected across time for all subjects (e.g., patients participating in the clinical trial). As noted above, interpolation (e.g., linear interpolation) may be employed to match quantitative clinical records, such as tumor size, to omics sample time points.

At step 420, quality control steps are performed on the merged data in some embodiments. The quality control steps can include some or all of reconciling duplicated clinical records and resolving discrepancies across data sources. In some embodiments, all such inconsistences and their resolutions are recorded in log files (for example, by the integration module 330). In some embodiments, this step may be omitted or combined with other quality control steps.

At step 422, the merged data is filtered, where samples for time points in which corresponding clinical information is missing are identified and removed from the merged data. In some embodiments this step may be omitted or combined with other steps.

At step 424, the merged data is sliced to generate two or more data sets (slices) using one or more criteria based on the clinical data to form sliced data sets. The data may be sliced multiple times to form multiple sliced data sets using different criteria. Various criteria for slicing are described above with respect to step 108 of FIG. 1. Exemplary data slices are listed below in Example 2.

At step 426, Bayesian causal relationship networks are generated that represent data underlying the sliced data sets. This can be described as “learning” a Bayesian network based on input data. Bayesian networks are cause-and-effect graphs that best describe the underlying correlation structure in the input data. These networks are composed of nodes and edges. Network nodes represent molecular features (proteins, lipids, metabolites), clinical variables (lab tests, tumor response), and patient demographics (treatment arm, age, race). Edges represent cause-and-effect relationships between network nodes.

Prior to Bayesian learning, each variable in the data slice is specified as middle, top, or bottom. This definition refers to the type of connections allowed for each variable. Middle variables are unconstrained in that they may serve as child or parent nodes. Top variables may only be parent nodes, thus they are constrained from serving as a child node. Conversely, bottom variables may be only child nodes, thus they are constrained from serving as parent nodes. In an example embodiment, the top variables consist of patient demographics and clinical interventions, such as trial arm assigned for Examples 1 and 2 discussed below. Bottom variables include features related to clinical outcome, such as tumor size and tumor response for Examples 1 and 2 discussed below. Lab tests and omic variables are considered as middle variables, thus allowing them to serve as parent or child nodes.

In some embodiments, the Bayesian network algorithm employed by the CTAW learns an ensemble of networks from each data slice with the ensemble of networks collectively representing the Bayesian network for the data slice. The number of networks to learn, in an example ensemble, may include 500 networks. In other embodiments, the number of networks learned by the CTAW in an ensemble may include 500-1000 networks. In yet other embodiments, the number of networks learned by the CTAW may include over 1000 networks. In some embodiments, Reconstructing Integrative Molecular Bayesian Networks (RIMBANet) is used as the platform for generating Bayesian Networks.

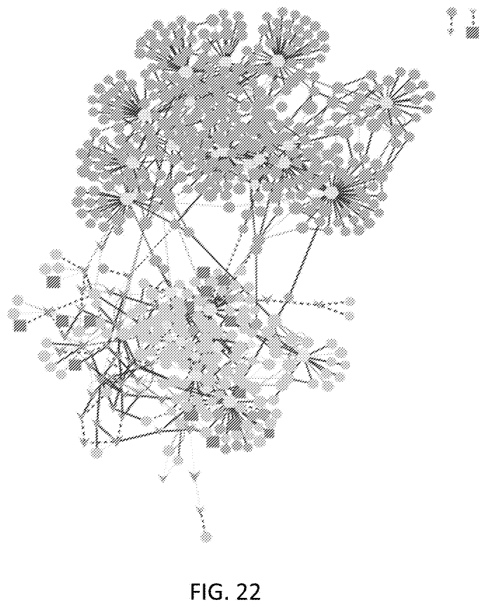

In some embodiments, following Bayesian learning, the following post-processing steps are applied. Any network in the ensemble in which fewer than 300 of the 500 networks converged is disregarded. Edges contained in any of the ensemble networks are combined, and the frequency of their occurrence is calculated. Edges that occurred infrequently across the ensemble of networks are removed by imposing an edge frequency requirement of 20%. The directionality of each edge is assigned for continuous variables by computing the Pearson correlation coefficient relating the parent node data set to the childe node data set. Edges that connect one or more discrete variables are considered “discrete.” Correlation coefficients greater than 0.2 are considered “direct”, while correlation coefficients less than −0.2 are considered “reverse.” Correlation coefficients that fail to be either “direct” or “reverse” are considered to be “causal.” A graphical representation of a network from an exemplary dataset is shown in FIG. 22. Further details regarding generation of the Bayesian causal relationship networks appears below in the section entitled “Generation of Bayesian Causal Relationship Networks using an AI-based System.” Further discussion and examples of generated Bayesian networks appear below in the section entitled “Output AI-Networks.”

In some embodiments, outcome drivers that are possible or potential biomarkers are identified by analyzing the topological features of each network learned by the CTAW 400. After a Bayesian causal relationship network is generated from a sliced data set, the topology of the network may be analyzed to indicate potential biomarkers for an outcome of interest. For example, a sliced data set including all patients may be used for generation of a Bayesian causal relationship network. In the Bayesian causal relationship network, a sub-network around an outcome variable of interest may be identified. For example, if the administered agent is intended to treat a condition causing solid tumors, the outcome variable of interest may be tumor size. The sub-network includes variables having a first degree relationship with the outcome variable of interest (e.g., variables directly connected to the tumor size variable by a relationship, which is shown as a variable connected to the tumor size variable by an “edge” in a graphical representation). The sub-network may also include variables having a second degree relationship with the outcome variable of interest (e.g., a variables connected by a relationship to a variable connected by a relationship with the tumor size variable). In some embodiments, the sub-network may also include variables having a third degree relationship with the outcome variable of interest. The variables in the sub-network are then analyzed as possible or potential biomarkers for the outcome of interest (e.g., for responsivity to treatment by the agent). For example, simulation may be employed using the Bayesian causal relationship network to probe the effect of the variables in the sub-network on the outcome variable of interest (e.g., tumor size).

In some embodiments, the data may be sliced by responsive and non-responsive patients and Bayesian causal relationship networks generated based on these sliced data sets. A sub-network may be identified around an outcome variable of interest in the Bayesian causal relationship network based on the responsive patient data. For example, a local network may be identified around the tumor size variable for the Bayesian causal relationship network based on responsive patient data.

The Bayesian relationship networks for responsive patients and for non-responsive patients may be compared with differences highlighting potential biomarkers for responsivity. In some embodiments, such a comparison may include the formation of a differential (delta) network based on the Bayesian relationship networks for the responsive patients and for the non-responsive patients. Further details regarding generation differential (delta) networks appear in the section below entitled “Generation of Bayesian Causal Relationship Networks using an AI-based System.”

Additionally, in some embodiments, a literature search is performed for each node by itself and in combination with the terms “cancer” or “mitochondria.” In some embodiments, nodes with more than 200 publications are removed from the sets of possible biomarkers because these nodes will not contribute to discovery of novel drug treatments or interactions.

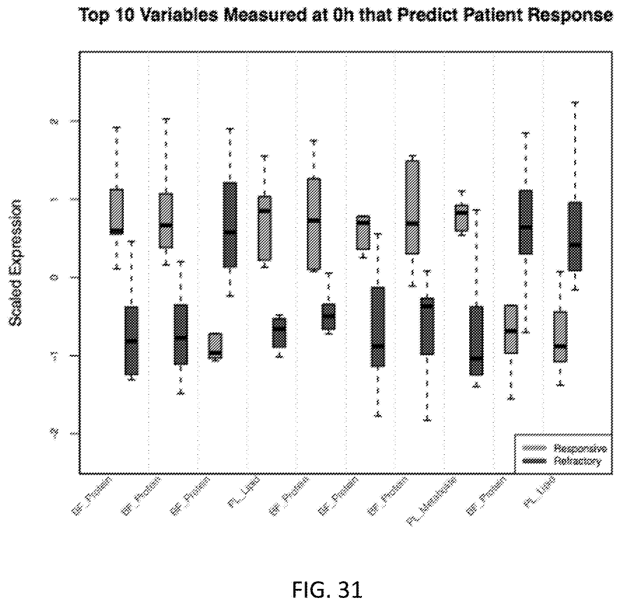

At step 432, companion diagnostic markers (CDx) are identified. CDx are biomarkers or potential biomarkers for a clinical outcome related to administration of an agent. CDx may be measured at any time prior to therapy or after the trial begins to predict patient outcome. Specifically, CDx markers are a panel of molecular features and/or lab tests that may be used to make predictions regarding the outcome of patients treated with an agent. Ideally, CDx used in a panel will be predictive or highly correlated with the outcome of interest and relatively uncorrelated with each other (e.g., orthogonal). CDx markers have three components (1) a set of which features that should be measured, (2) a time point in which the features are to be measured, and (3) a clinical output to predict. For example, a scenario in which CDx markers are derived to predict patient outcome is as follows. The panel of markers to be measured consists of the levels of seven proteins measured in buffy coat, two lipids measured in plasma, and one metabolite measured in plasma. The time point of measurement is immediately before beginning the first administration of an agent (e.g., immediate before a first infusion of CoQ10). The predictive power for these CDx markers are to use these molecular features to predict if patients would be responsive or refractory to treatment, where length of time enrolled on trial is taken to be a surrogate for patient response. The resulting set of CDx markers may be visualized as a boxplot, as shown in FIG. 31.

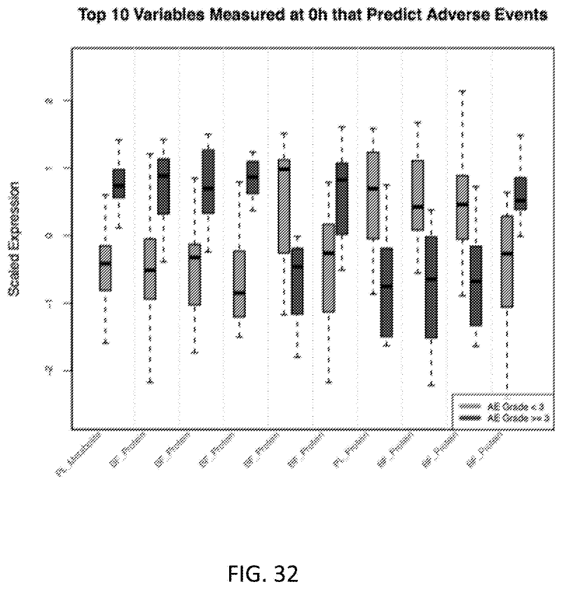

Similarly, CDx markers may be found to predict severe adverse events. Here, the panel of CDx markers may consist of one protein measured in plasma, one metabolite measured in plasma, and eight proteins measured in buffy coat. By measuring these CDx markers prior to the start of therapy, a set of patients who experience severe adverse events may be predicted as well as the remaining patients who are predicted not to experience severe adverse events. FIG. 32 shows CDx markers that predict adverse events.

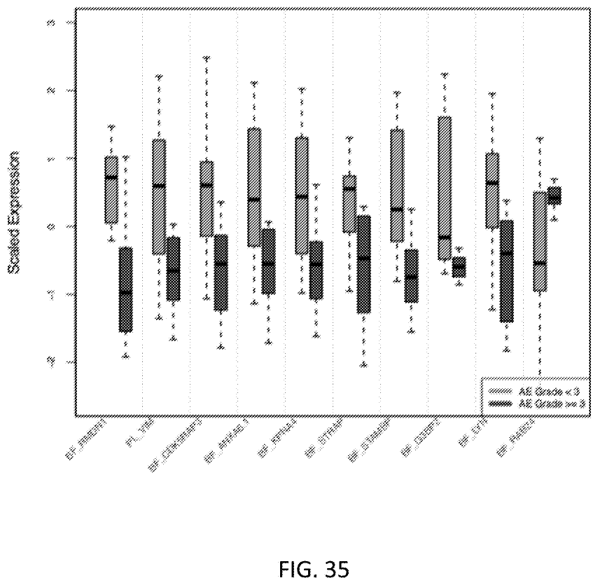

As used herein, companion diagnostics (CDx) are potential biomarkers or biomarkers for a clinical outcome related to administration of an agent. Patient outcome may be defined for example by differentiating patients that had an overall clinical benefit from patients that exhibited no clinical benefit, or by differentiating patients who experienced adverse events from those who do not. In this example method 400, analysis of data sets sliced by patients that exhibited an overall clinical benefit 428 and patients that exhibited no clinical benefit 430 is used to identify CDx biomarkers that predict patient response to administration of the agent. The CTAW may be used to identify a set of CDx markers that predict patient outcome prior to the start of therapy. In some embodiments, CDx or candidate CDx are identified using topological features of the generated causal relationship networks. In some embodiments, candidate CDx are identified using a combination of network topological features and statistical analysis. Candidate CDx markers are possible biomarkers, from which CDx potential biomarkers are identified. For example, candidate CDx markers may be found to predict if patients experience severe adverse events. FIG. 35 illustrates a boxplot for the top 10 candidate CDx markers determined from differential expression.

In some embodiments CDx are identified using a combination of network topological features (e.g., to determine outcome drivers), statistical analysis (e.g., to find differentially expressed variables), and machine learning methods.

In some embodiments, network topological features and statistical analysis are used to identify sets of possible biomarkers (e.g., candidate CDx markers) and machine learning is used to analyze the sets of possible biomarkers to select a subset that are relatively uncorrelated with each other, but strongly correlated or predictive of the outcome, which are the CDx markers. For example, in one such embodiment, the steps involved in identifying CDx markers are (1) harvest variables that are drivers of key outputs related to the prediction objective in the relevant AI networks; (2) identify differentially expressed variables between the patient stratification groups at the specified time point; and (3) input the results from steps (1) and (2) into a machine learning algorithm (e.g., regression using an elastic net) that determines which features robustly predict phenotypic outcome. Further discussion of the analysis to determine the companion diagnostics is presented below in the section “Determination of Potential Biomarkers (e.g., Companion Diagnostics).”

Turning again to FIG. 4, following the CDx pipeline, at step 434, quality control steps ensure the reliability of the identified biomarkers by confirming their measured values in the processed data set that was input to the CDx pipeline. In some embodiments these quality control steps 434 may be omitted or combined with other steps. In some embodiments, the first step in the quality control procedure is to randomly select ten candidate CDx markers. For the candidate CDx markers selected for quality control, summary statistics (mean and standard deviation) are computed for the patient stratification groups (such as patients who experienced adverse events, and patients who did not experience adverse event). The calculated summary statistics are then compared to the values computed previously by the CTAW pipeline to ensure that the correct data points are being selected and the proper processing steps are being applied. In addition, a detailed quality control report is generated for a given CDx analysis.

Omics Data Processing

Buffy Coat and Plasma Proteomics Data Processing

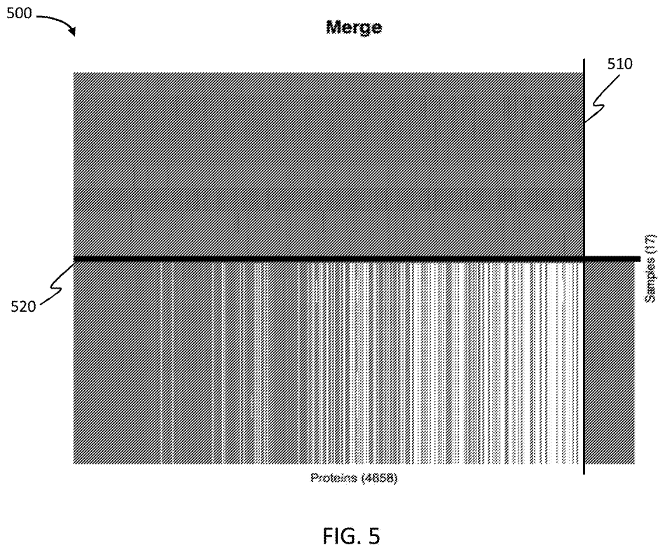

In some embodiments, buffy coat and plasma proteomics data files are processed according to the following methodology, which will use the term “proteomics” as referring to either sample type. In some embodiments, the processed buffy coat and plasma proteomics data are provided as proteomics data 406 to the CTAW 400. In some embodiments, data processing begins with proteomics data files that have been annotated by a parsing tool to ensure compatibility with the CTAW 400. Annotated data collected across multiple batches are then merged to create a single data frame 500, as shown in FIG. 5, containing all proteins measured in any of the collected samples. In FIG. 5 samples present in two raw data files are separated by horizontal line 520. Proteins measured uniquely in one raw data file but not the other separated by the vertical line 510.

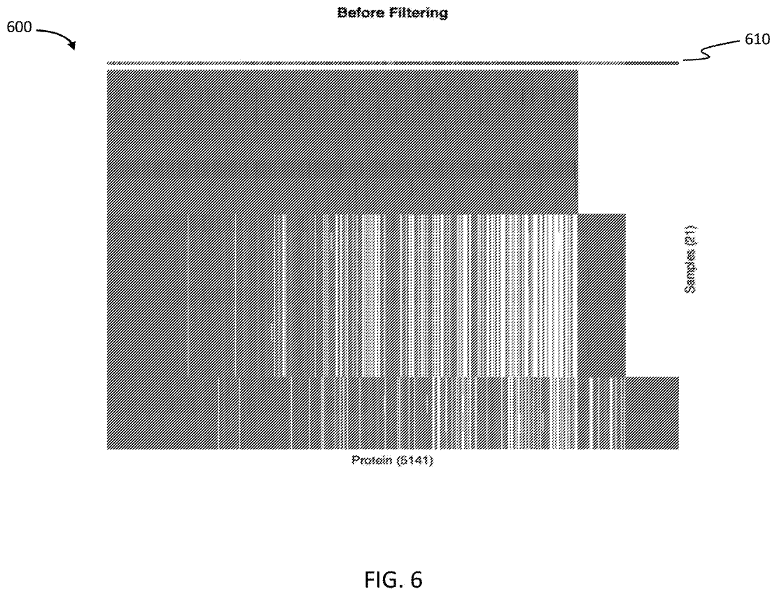

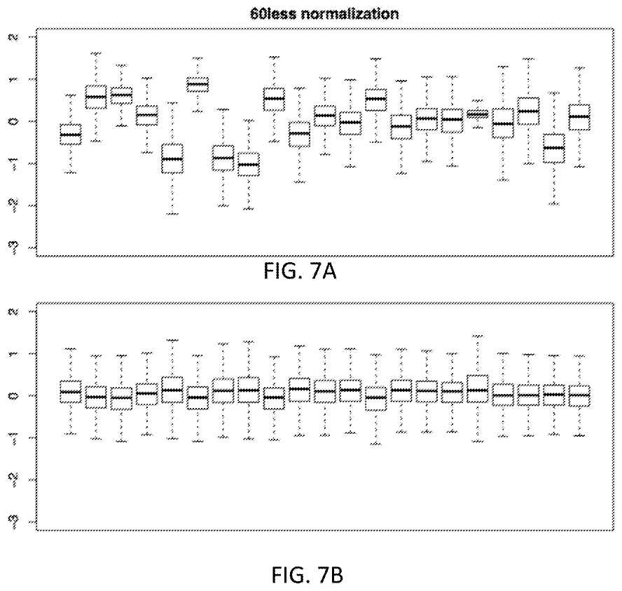

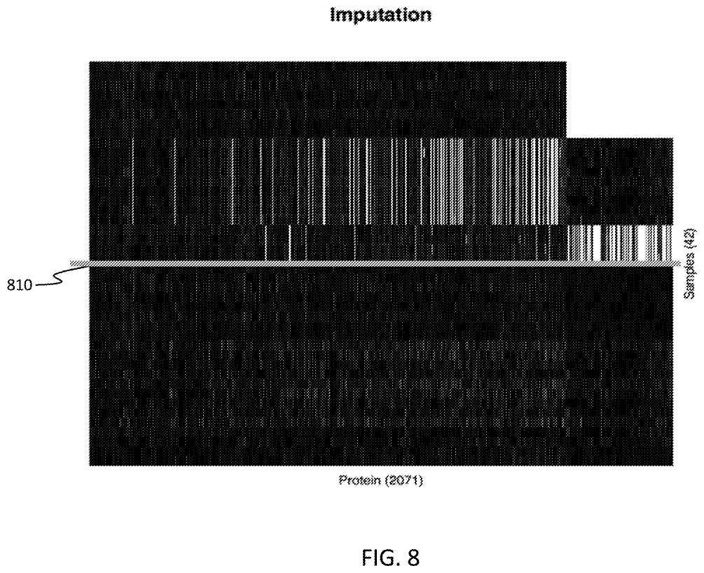

In some embodiments, proteomics data is transformed by applying log₂ transformation. Protein identifiers that had been measured more than once are summarized by their median value, ensuring that only unique protein identifiers remain. In some embodiments, proteins that had missing values in more than 60% of samples were considered unreliable, and therefore removed from further analysis, as shown in the data representation 600 in FIG. 6. In FIG. 6, retained and removed proteins are indicated by lighter and darker shades of gray in the top row 610, respectively. In some embodiments, when processing buffy coat proteomics samples, an additional filtering step (QCP filtering) is applied that ensures protein levels are measured relative to their QCP samples consistently. In some embodiments, data is normalized by an approach called 60-less that involves first, computing the coefficient of variation for each feature, and next, considering features in the bottom 60% coefficient of variation to be invariant. Then each sample is centered by the median of the invariant proteins, and scaled by mean interquartile range (IQR) divided by the inter quartile range for each sample. The protein distribution across samples is shown in FIG. 7A before the normalization process (60-less approach). FIG. 7B illustrates the protein distribution across samples after the normalization process is applied. Missing values are imputed using a script, program or software code that automatically samples uniformly from two standard deviations below its mean and two standard deviations above its mean. FIG. 8 illustrates a data set before and after imputation, where missing data in the normalized proteomics data set is imputed. A data set before imputation is presented above line 810, and the corresponding data set after imputation is presented below line 810.

Structural Lipidomics

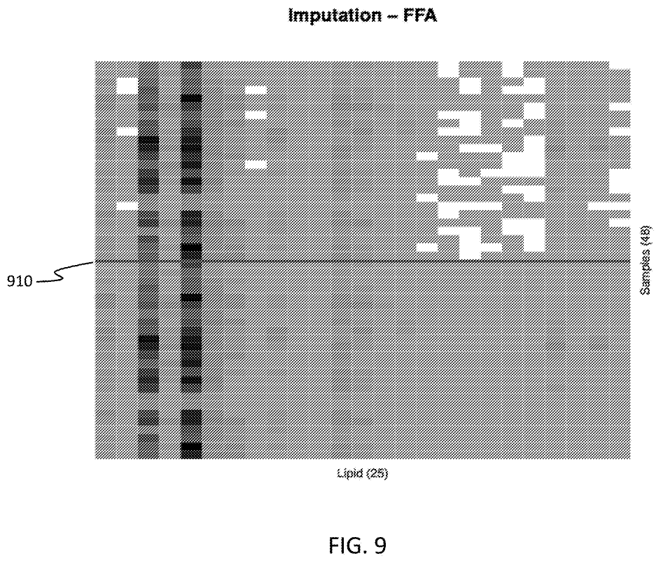

In some embodiments, structural lipidomics data files are annotated by a parsing tool to convert the raw data to a format that is compatible with the CTAW 400. The processed lipidomics data may be provided to the CTAW 400 as lipidomics data 402. In some embodiments, data processing begins by performing imputation on missing data found in individual lipidomics data files. In some embodiments, missing values are imputed by sampling uniformly between the lowest value observed in any lipid class and half its value. FIG. 9 illustrates a data set before and after imputation. The data set before imputation is shown above horizontal line 910, and the data set after imputation is shown below the horizontal line 910. In some embodiments, imputation is performed on a per-data file basis so that imputation is relative to the minimum values observed in each lipidomics data run.

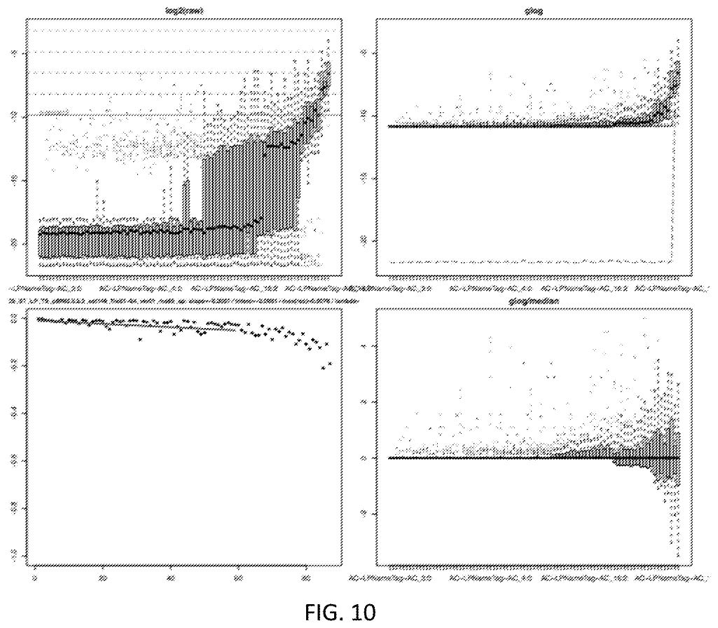

Following imputation, data files are merged into a single list of lipid classes, and log₂ transformed. In some embodiments, normalization is undertaken per-lipid class where an optimal lambda (λ) value is determined for each class, lipid values in this class are transformed by glog transformation, and transformed lipids are median centered. Data sets after each step of the normalization process are illustrated in FIG. 10. Next, any lipid that contains missing data is removed because the presence of missing data indicates lipids that were not detected consistently across batches. Finally, any lipids that were previously found to be unstable are removed thus ensuring the robustness of the processed data set.

Plasma Signaling Lipidomics

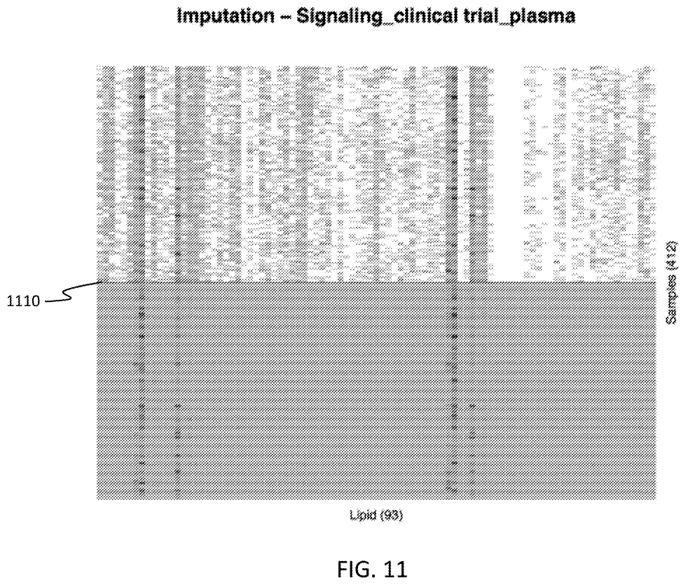

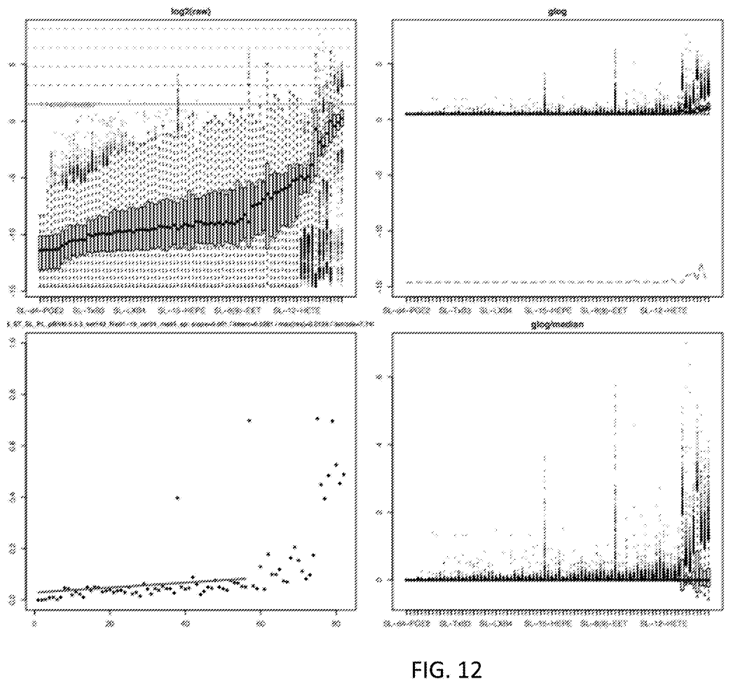

In some embodiments, signaling lipidomics files are annotated by a parsing tool to convert the raw data to a format that is compatible with the CTAW 400. The processed lipidomics data may be provided to the CTAW 400 as lipidomics data 402. In some embodiments, any missing data present in individual lipid files is imputed by uniform sampling between the lowest value observed in each file, and half this value. The imputed data set is illustrated in FIG. 11, in which, the data set before imputation is shown above the horizontal line 1110, and the data set after imputation is shown below the horizontal line 1110. This imputation is performed on a per-data file basis, ensuring that the imputed data lies within the range appropriate to each lipidomics run. In some embodiments, after imputation, data is merged and any lipid not measured in across all samples in a batch is removed. In some embodiments, data is then log₂ transformed, and normalized by determining an optimal lambda (λ) value, applying glog transformation, and median centering. Data sets after each step of the normalization process are illustrated in FIG. 12. In some embodiments, following normalization, any lipids that were previously flagged as unstable are removed.

Urine Proteomics

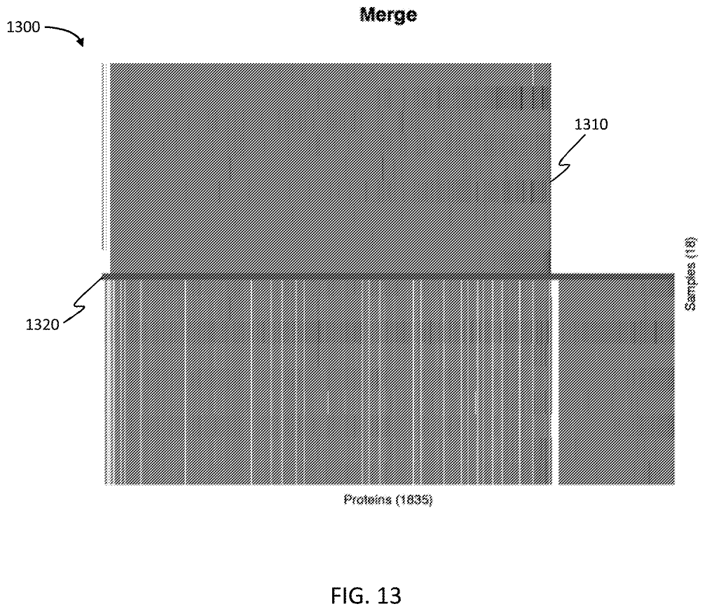

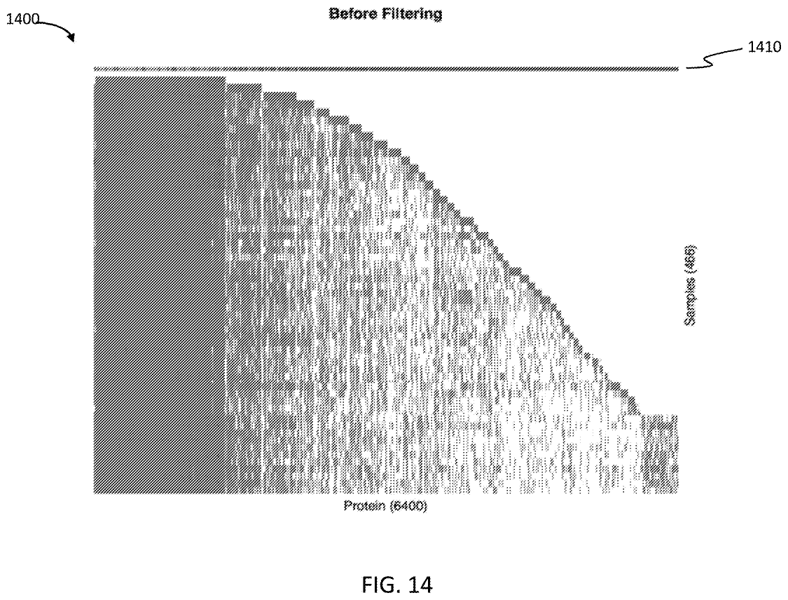

In some embodiments, data processing begins with proteomics data files that have been annotated by a custom parsing tool to ensure compatibility with the CTAW 400. The processed proteomics data may be provided to the CTAW 400 as proteomics data 406. In some embodiments, annotated data collected across multiple batches are then merged to create a single data frame 1300, as shown in FIG. 13, containing all proteins measured in any of the collected samples. In FIG. 13, samples present in two raw data files are separated by the horizontal line 1320. Proteins measured uniquely in one raw data file but not the other are separated by the vertical line 1310. In some embodiments, proteins that had missing values in more than 75% of samples are considered unreliable, and therefore removed from further analysis as shown in the data representation 1400 in FIG. 14. In FIG. 14, retained and removed proteins are indicated by the light gray and the dark gray in the top row 1410, respectively.

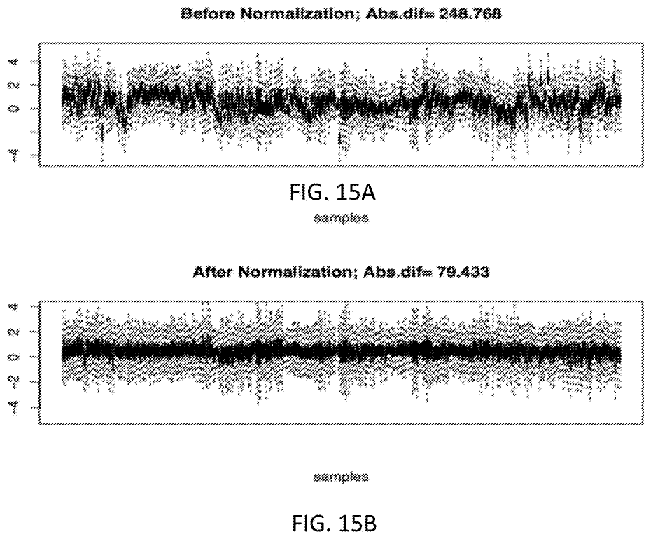

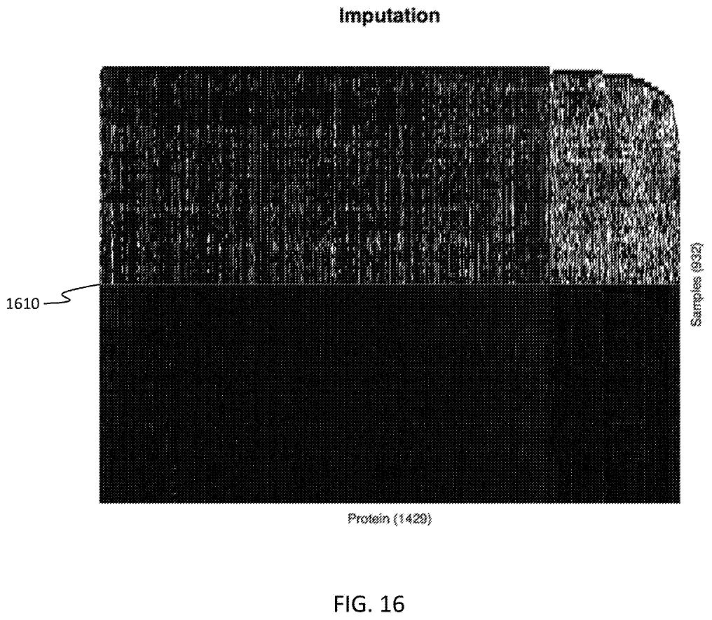

In some embodiments, urine proteomics data is normalized by a procedure designed to reduce the variability arising from differences in hydration. This is accomplished by identifying stable proteins whose values depend on dilution level only, and are thus highly correlated with each other and detectable in each urine sample. The first step in identifying stable proteins is to consider proteins that are present in more than 97% of urine samples. Next, hierarchical clustering is applied to this set of candidate stable proteins using multiscale bootstrap resampling to estimate the significance of each cluster in the clustering result. Clusters are then combined, and their members' ability to serve as a set of stable urine proteins is evaluated by computing the sum of absolute deviation between the normalized values and the average normalized value. The optimal set of stable urine proteins is selected to be the set that produced the smallest sum of absolute deviation. Given this set of stable urine proteins, a multiplier is calculated by computing the median value of stable proteins across samples, dividing the expression level of each stable protein by this value, and computing the average expression of stable proteins per sample. The resulting value serves as a divisor to be applied per-sample to all urine protein values, which produces the normalized urine proteomics data. The protein distribution across samples is shown in FIG. 15A before the normalization process. FIG. 15B illustrates the protein distribution across samples after the normalization process is applied. The “abs. dif” value in FIGS. 15A and 15B refers to the sum of absolute deviation between the values and the average value for the raw data and normalized data, respectively. Following normalization, protein values are log₂ transformed. In some embodiments, the missing data in the normalized proteomics data flow is then imputed. FIG. 16 illustrates a data set before and after imputation, where missing values are imputed by sampling uniformly from two standard deviations below its mean and two standard deviations above its mean. The data set before imputation is presented above line 1610, and the data set after imputation is presented below line 1610.

Plasma Metabolomics

In some embodiments, plasma metabolomics data is obtained via three different techniques, depending upon the procedure (chromatography) performed on the sample before it is analyzed using a spectrometer. These three techniques are liquid chromatography-tandem mass spectrometry (LCMSMS), liquid chromatography-mass spectrometry (LCMS) and gas chromatography-mass spectrometry (GCMS). Plasma metabolomics data files from each of the techniques are processed independently according to following methodology and merged in the end. The processed metabolomics data may be provided to the CTAW 400 as metabolomics data 404. Data processing begins with metabolomics data files that have been annotated by custom parsing tools to ensure compatibility with the CTAW 400.

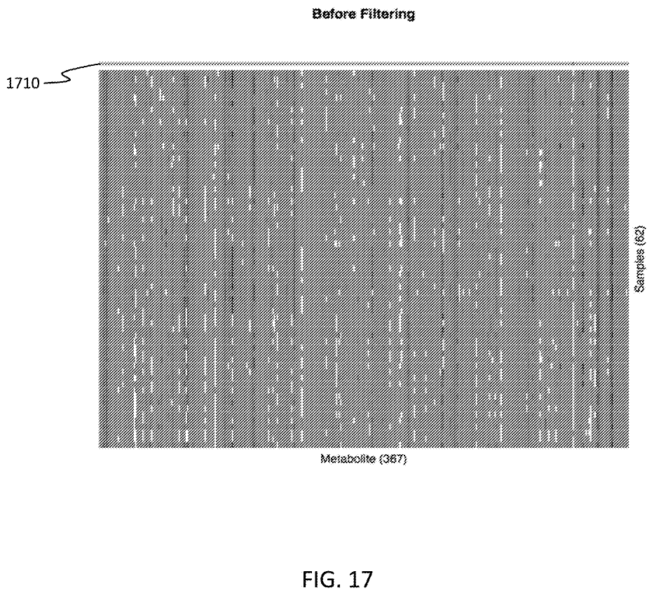

In some embodiments, annotated data collected across multiple batches are then merged to create a single data frame containing all metabolites measured in any of the collected samples for a particular procedure. In some embodiments, metabolite names are replaced with a unique identifier which may be retrieved from a metabolomics database. In some embodiments, metabolites having missing values in more than 60% of samples are considered unreliable, and therefore removed from further analysis, as shown in the data representation 1700 in FIG. 17. In FIG. 17, retained and removed metabolites are indicated by the light gray and dark gray in the top row 1710, respectively.

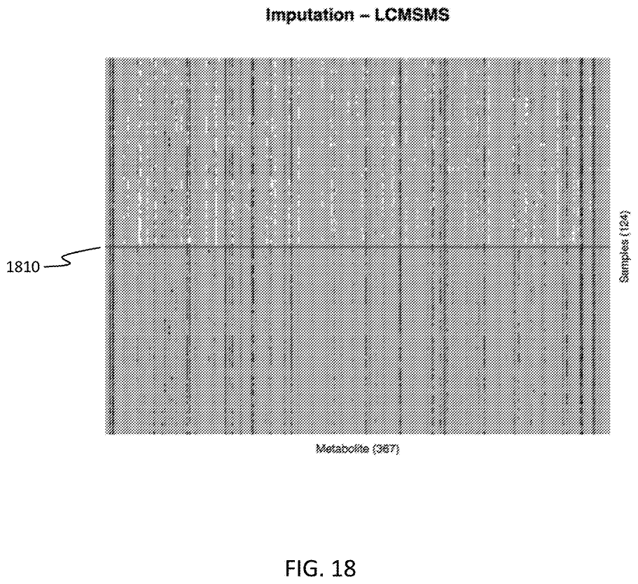

In some embodiments, any metabolite that contains missing values has its missing values imputed by sampling uniformly from two standard deviations below its mean and two standard deviations above its mean. The imputed data set is illustrated in FIG. 18, in which the data set before imputation is shown above the horizontal line 1810, and the data set after imputation is shown below the horizontal line 1810.

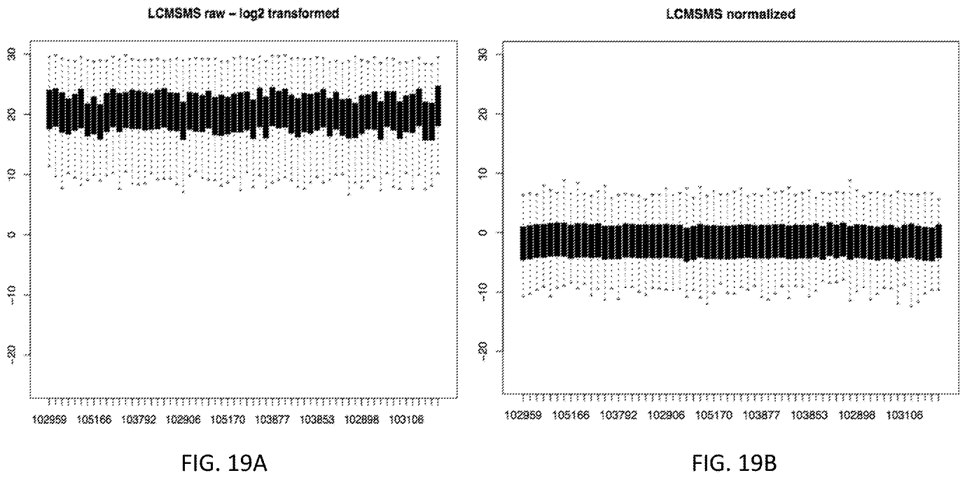

In some embodiments, metabolomics data is transformed by applying log₂ transformation. In some embodiments, data is normalized using an approach called 60-less that involves first, computing the coefficient of variation for each feature, and next considering features in the bottom 60% coefficient of variation to be invariant. Then, each sample is centered by the median of the invariant metabolite, and scaled by mean interquartile range (IQR) divided by the inter quartile range for each sample. The metabolite distribution across samples is shown in FIG. 19A before the normalization process (60-less approach). FIG. 19B illustrates the metabolite distribution across samples after the normalization process is applied.

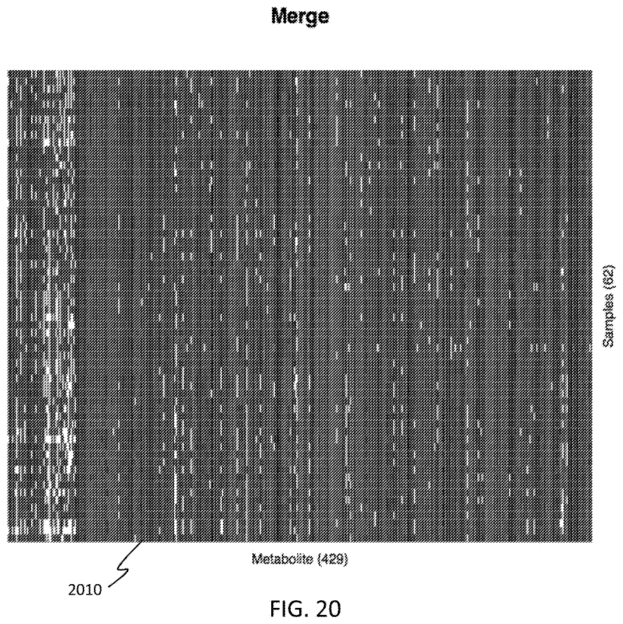

After normalization, metabolite data from all three techniques are merged together. The resulting data set is illustrated in FIG. 20, in which samples present in two normalized data files are separated by the vertical line 2010. Metabolites measured uniquely in one raw data file but not the other separated by the vertical line 2010. In some embodiments, a metabolite identifier/metabolite measured in more than one technique is filtered according to priority. The priority for metabolites across techniques is as follows: LCMSMS>LCMS>GCMS. Thus, if a metabolite identifier/ metabolite is present in LCMSMS and LCMS dataset then its LCMS values are filtered ensuring that only one set of value per metabolite identifier exists.

Omics Data Consolidation

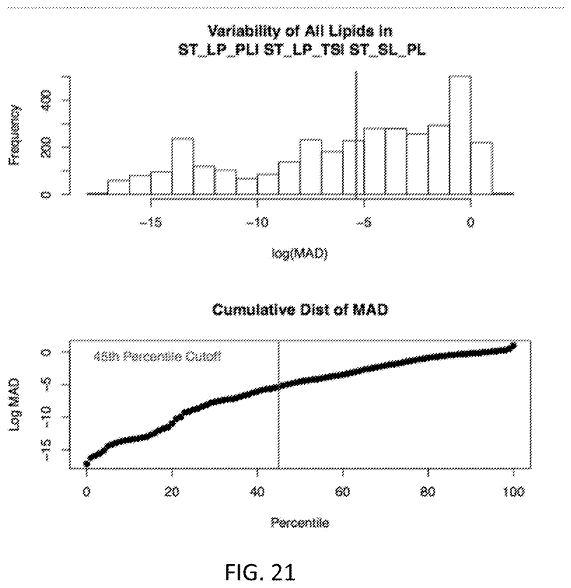

In some embodiments, processed-molecular features measured by omics technologies are combined into a list. Replicated samples are averaged so that only unique samples are retained. To avoid including lipids with a low variability due to excessive missing data, invariant lipids are removed, as illustrated in FIG. 21. Following this filtering, omics samples are annotated with phenotypic information regarding the time of collection and merged into a single data frame.

Input of Raw Omics Data

In some embodiments, users (e.g., clinical trial administrators) deposit raw omic data into a secure shared drive, and these data files are evaluated for processing by the CTAW 400. The system described herein identifies which files contain data and annotates the data files with their omic technology, sample type and batch. The approach begins by assuming that all files present in the shared drive are valid data files, unless their file name contains any blacklisted keywords. Table 1 (below) lists the file names containing blacklist terms that are excluded. Additionally, merged proteomics raw file, designated by the suffix “all” or “all-annotated,” is disregarded if the individual files are also present.

TABLE 1
File names containing blacklist terms are excluded.

| Key Words | Rationale |
|---|---|
| .docx, .db, .tmp, .zip | Raw omic data files do not contain these |
| | file extensions |
| Condition reference, | Descriptive files that do not contain data |
| sample list, definition | |
| DoD, BP0312-01 | Data corresponding to other omics projects |
| Peptide, Protein peptide | Peptide-level proteomics are not processed |

After valid raw omic data files are identified, symbolic links are created with coded names that specify the omics technology used and the sample type corresponding to each raw data file. The omic technology corresponding to each file is identified according to keywords present in the original file name or by the presence of features unique to individual technologies; whereas, the sample type is determined primarily by the presence of key words in the file name (urine, plasma, tissue, or buffy coat). In instances where the sample type cannot be determined from the file name, the sample type is identified by looking up the present samples in the master file. Following the data-type identification, symbolic links are created. Table 2 (below) illustrates an exemplary symbolic link analyzed by the system described herein. The exemplary symbolic link is 105_ST_LP_CT_UR_169_02_01.xlsx.

TABLE 2
Nomenclature of symbolic links. A symbolic link, such as
105_ST_LP_CT_UR_169_02_01.xlsx, contains
eight positions of annotation information
delimited by underscores.

| Position | Value | Description | Constant |
|---|---|---|---|
| 1 | 105 | Analysis number | Yes |
| 2 | ST | Solid tumor | Yes |
| 2 | PT (proteomics), LP | Omic technology | No |
| | (lipidomics), SL | | |
| | (signaling lipidomics), | | |
| | MG (metabolomics) | | |
| 4 | CT | Clinical trial | Yes |
| 5 | PL (plasma), BF (buffy | Sample type | No |
| | coat), TS (tissue), UR | | |
| | (urine) | | |
| 6 | Integer, one to the | Folder number | No |
| | number of data folders | | |
| 7 | Integer, one to the | File number | No |
| | number of files present | | |
| | in folder | | |
| 8 | 01 | Version | Yes |

Input Clinical Records Data

In some embodiments, clinical data is input into the CTAW 400 as a series of comma-separated value (CSV) files. Table 3 below illustrates exemplary input clinical data files. The input data files follow the Study Data Tabulation Model (SDTM) defined by the Clinical Data Interchange Standards Consortium (CDISC).

TABLE 3
Clinical Data Files as inputs into the Clinical Trial Analytics Workflow.

| | | | Analyzed |
|---|---|---|---|
| CDISC Domain | | | by |
| model | File Name | Description | CTST |
| Events | ae.csv | Adverse Events | Yes |
| Interventions | cm.csv | Concomitant | No |
| | | Medications | |
| Special-purpose | co.csv | Comments | No |
| Special-purpose | dm.csv | Demographics | Yes |
| Events | ds.csv | Disposition | Yes |
| Events | dv.csv | Protocol Deviations | No |
| Interventions | ex.csv | Exposure | Yes |
| Findings | fa.csv | Findings About Events | Yes |
| | | or Interventions | |
| Findings | ie.csv | Inclusion/Exclusion | No |
| | | Exceptions | |
| Findings | lb.csv | Laboratory Tests | Yes |
| Events | mh.csv | Medical History | No |
| Findings | pc.csv | Pharmacokinetics | No |
| | | Concentrations | |
| Findings | pe.csv | Physical Examinations | No |
| Findings | qs.csv | Questionnaires | Yes |
| Special-Purpose | relrec.csv | Relate Records | No |
| Relationship | | | |
| Oncology | rs.csv | Tumor Response | Yes |
| Findings | sc.csv | Subject Characteristics | Yes |
| Findings | suppe.csv | Supplement to | No |
| | | Physical Examinations | |
| Interventions | suppcm.csv | Supplement to | No |
| | | Concomitant | |
| | | Medications | |
| Special-purpose | suppdm.csv | Supplement to | No |
| | | Demographics | |
| Events | suppds.csv | Supplement to | No |
| | | Disposition Events | |
| Events | suppdv.csv | Supplement to | No |
| | | Protocol Deviations | |
| Interventions | suppex.csv | Supplement to | No |
| | | Exposure | |
| Findings | suppfa.csv | Supplement to | No |
| | | Findings About | |
| Findings | supplb.csv | Supplement to | No |
| | | Laboratory Exams | |
| Events | suppmh.csv | Supplement to Medical | No |
| | | History | |
| Events | suppae.csv | Supplement to Adverse | No |
| | | Events | |
| Oncology | supptr.csv | Supplement to Tumor | No |
| | | Results | |
| Oncology | supptu.csv | Supplement to Tumor | No |
| | | Identification | |
| Special-Purpose | sv.csv | Subject Visits | No |
| Oncology | tr.csv | Tumor Results | Yes |
| Trial Design | ts.csv | Trial Summary | No |
| Oncology | tu.csv | Tumor Identification | No |
| Findings | vs.csv | Vital Signs | No |

Generation of Molecular Profile Data

Systems and methods for generating molecular profile data from patient samples may include systems and methods for mass spectrometry based proteomics, microarray gene expression, qPCR gene expression, mass spectrometry based metabolomics, and mass spectrometry based lipidomics, SNP microarrays, and other platforms and technologies. Large-scale high-throughput quantitative proteomic analysis may be employed to analyze the patient samples.

In some embodiments, quantitative polymerase chain reaction (qPCR) and proteomics are performed to profile changes in cellular mRNA and protein expression by quantitative polymerase chain reaction (qPCR) and proteomics. Total RNA can be isolated using a commercial RNA isolation kit. Following cDNA synthesis, specific commercially available qPCR arrays (e.g., those from SA Biosciences) for disease area or cellular processes such as angiogenesis, apoptosis, and diabetes, may be employed to profile a predetermined set of genes by following a manufacturer's instructions. For example, the Biorad cfx-384 amplification system can be used for all transcriptional profiling experiments. Following data collection (Ct), the final fold change over control can be determined using the δCt method as outlined in manufacturer's protocol. Proteomic sample analysis can be performed as described in subsequent sections.

There are numerous art-recognized technologies suitable for this purpose. An exemplary technique, iTRAQ analysis in combination with mass spectrometry, is briefly described below.

The quantitative proteomics approach is based on stable isotope labeling with the 8-plex iTRAQ reagent and 2D-LC MALDI MS/MS for peptide identification and quantification. Quantification with this technique is relative: peptides and proteins are assigned abundance ratios relative to a reference sample. Common reference samples in multiple iTRAQ experiments facilitate the comparison of samples across multiple iTRAQ experiments.

For example, to implement this analysis scheme, six primary samples and two control pool samples can be combined into one 8-plex iTRAQ mix according to the manufacturer's suggestions. This mixture of eight samples then can be fractionated by two-dimensional liquid chromatography; strong cation exchange (SCX) in the first dimension, and reversed-phase HPLC in the second dimension, then can be subjected to mass spectrometric analysis.

A brief overview of exemplary laboratory procedures that can be employed is provided herein.

Protein extraction: Cells can be lysed with 8 M urea lysis buffer with protease inhibitors (Thermo Scientific Halt Protease inhibitor EDTA-free) and incubate on ice for 30 minutes with vertex for 5 seconds every 10 minutes. Lysis can be completed by ultrasonication in 5 seconds pulse. Cell lysates can be centrifuged at 14000×g for 15 minutes (4° C.) to remove cellular debris. Bradford assay can be performed to determine the protein concentration. 100 μg protein from each samples can be reduced (10 mM Dithiothreitol (DTT), 55° C., 1 h), alkylated (25 mM iodoacetamide, room temperature, 30 minutes) and digested with Trypsin (1:25 w/w, 200 mM triethylammonium bicarbonate (TEAB), 37 oC, 16 h).

iTRAQ 8 Plex Labeling: Aliquot from each tryptic digests in each experimental set can be pooled together to create the pooled control sample. Equal aliquots from each sample and the pooled control sample can be labeled by iTRAQ 8 Plex reagents according to the manufacturer's protocols (AB Sciex). The reactions can be combined, vacuumed to dryness, re-suspended by adding 0.1% formic acid, and analyzed by LC-MS/MS.

2D-NanoLC-MS/MS: All labeled peptides mixtures can be separated by online 2D-nanoLC and analysed by electrospray tandem mass spectrometry. The experiments can be carried out on an Eksigent 2D NanoLC Ultra system connected to an LTQ Orbitrap Velos mass spectrometer equipped with a nanoelectrospray ion source (Thermo Electron, Bremen, Germany)

The peptides mixtures can be injected into a 5 cm SCX column (300 μm ID, 5 μm, PolySULFOETHYL Aspartamide column from PolyLC, Columbia, Md.) with a flow of 4 μL/min and eluted in 10 ion exchange elution segments into a C18 trap column (2.5 cm, 100 μm ID, 1 μm, 300 Å ProteoPep II from New Objective, Woburn, Mass.) and washed for 5 min with H2O/0.1% FA. The separation then can be further carried out at 300 nL/min using a gradient of 2-45% B (H2O/0.1% FA (solvent A) and ACN/0.1% FA (solvent B)) for 120 minutes on a 15 cm fused silica column (75 μm ID, 5 μm, 300 Å ProteoPep II from New Objective, Woburn, Mass.).

Full scan MS spectra (m/z 300-2000) can be acquired in the Orbitrap with resolution of 30,000. The most intense ions (up to 10) can be sequentially isolated for fragmentation using High energy C-trap Dissociation (HCD) and dynamically exclude for 30 seconds. HCD can be conducted with an isolation width of 1.2 Da. The resulting fragment ions can be scanned in the orbitrap with resolution of 7500. The LTQ Orbitrap Velos can be controlled by Xcalibur 2.1 with foundation 1.0.1.

Peptides/proteins identification and quantification: Peptides and proteins can be identified by automated database searching using Proteome Discoverer software (Thermo Electron) with Mascot search engine against SwissProt database. Search parameters can include 10 ppm for MS tolerance, 0.02 Da for MS2 tolerance, and full trypsin digestion allowing for up to 2 missed cleavages. Carbamidomethylation (C) can be set as the fixed modification. Oxidation (M), TMT6, and deamidation (NQ) can be set as dynamic modifications. Peptides and protein identifications can be filtered with Mascot Significant Threshold (p<0.05). The filters can be allowed a 99% confidence level of protein identification (1% FDA).

The Proteome Discoverer software can apply correction factors on the reporter ions, and can reject all quantitation values if not all quantitation channels are present. Relative protein quantitation can be achieved by normalization at the mean intensity.

Generation of Bayesian Causal Relationship Networks using an AI-based System

Generating Bayesian causal relationship networks is explained in greater detail below with respect to an AI-based informatics system solely for illustrative purposes. However, one of ordinary skill in the art will recognize that other systems employing Bayesian analysis could be employed.

Generation of Bayesian causal relationship networks based on sliced data sets may be performed using an artificial intelligence (AI)-based informatics system or platform. In an example embodiment, the AI-based system employs mathematical algorithms to establish causal relationships among the input variables (e.g., the processed clinical records data and the processed molecular profile data). This process is based only on the input data alone, without taking into consideration prior existing knowledge about any potential, established, and/or verified biological relationships. As noted above, further details regarding generation of Bayesian causal relationship networks from biological data appears in U.S. Patent Application Publication No. US2012/0258874A1 entitled, “Interrogatory Cell-Based Assays and Uses Therof,” the entire contents of which is incorporated by reference herein.

In some embodiments, a significant advantage of such AI-based systems for generation of Bayesian causal relationship networks is that the resulting networks are based solely on the sliced data without resorting to or taking into consideration any existing knowledge in the art concerning the biological process. Further, preferably, no data points are statistically or artificially cut-off and, instead, all sliced data is fed into the AI-system for determining associations among the variables. Accordingly, the resulting statistical models in the form of Bayesian causal relationship networks generated are unbiased, because they do not take into consideration any known biological relationships among the input data.

Specifically, a sliced data set is input into the AI-based information system, which builds a statistical model based on data associations. Simulation-based networks are then derived from the statistical model.

The sliced data is normalized, if needed, and input into the AI-based informatics system (e.g., Bayesian network module 350) as an input data set. In some embodiments, the AI-based informatics system uses input data is used to construct a library or list of potential network fragments that define quantitative relationships among small sets (e.g., 2-3 member sets or 2-4 member sets) of input data. The different types of input data are termed “variables” regardless of whether they may vary in an individual patient. For example, gender, age, ethnicity, blood pressure, and expression level of a particular protein would all be termed “variables” in this context. The relationships between the variables in a network fragment may be linear, logistic, multinomial, dominant or recessive homozygous, etc. The relationship in each fragment is assigned a Bayesian probabilistic score that reflects how likely the candidate relationship is given the input data, and also penalizes the relationship for its mathematical complexity. The most likely fragments in the library can be identified (the likely fragments) based on the score. Various model types may be used in fragment enumeration including but not limited to linear regression, logistic regression, (Analysis of Variance) ANOVA models, (Analysis of Covariance) ANCOVA models, non-linear/polynomial regression models and even non-parametric regression. The prior assumptions on model parameters may assume Gull distributions or Bayesian Information Criterion (BIC) penalties related to the number of parameters used in the model.

In a network inference process, an ensemble of initial trial networks is constructed with each network in the ensemble constructed from a subset of fragments in the fragment library or in a list of fragments and the initial trial networks are evolved. In some embodiments, each initial trial network in the ensemble of initial trial networks is constructed with a different subset of the fragments from the fragment library or the fragment list. Eventually an ensemble of initial trial networks is created (e.g., 500 networks or 1000 networks) from different subsets of network fragments in the library. This process may be termed parallel ensemble sampling. In some embodiments, each trial network in the ensemble is evolved or optimized by adding, subtracting and/or substitution additional network fragments from the library. In some embodiments, if additional data is obtained, the additional data may be incorporated into the network fragments in the library or on the list and may be incorporated into the ensemble of trial networks through the evolution of each trial network. After completion of the optimization/evolution process, the ensemble of trial networks may be described as the generated networks.

An overview of the mathematical representations underlying the Bayesian networks and network fragments, which is based on Xing et al., “Causal Modeling Using Network Ensemble Simulations of Genetic and Gene Expression Data Predicts Genes Involved in Rheumatoid Arthritis,” PLoS Computational Biology, vol. 7, issue. 3, 1-19 (March 2011) (e100105), is presented below.

A multivariate system with random variables X=X₁, . . . , Xₙ may be characterized by a multivariate probability distribution function P(X₁, . . . , Xₙ; Θ), that includes a large number of parameters Θ. The multivariate probability distribution function may be factorized and represented by a product of local conditional probability distributions:

$$P ( {X}_{1} , … , {X}_{n} ; Θ ) = {\prod }_{i - 1}^{n} {P}_{i} ( {X}_{i} \vert {Y}_{j 1} , … , {Y}_{{jK}_{i}} ; {Θ}_{i} ) ,$$

in which each variable Xᵢ is independent from its non-descendent variables given its Kᵢ parent variables, which are Yⱼ₁, . . . , Y_{jK}_{ᵢ} . After factorization, each local probability distribution has its own parameters Θᵢ.

The multivariate probability distribution function may be factorized in different ways with each particular factorization and corresponding parameters being a distinct probabilistic model. Each particular factorization (model) can be represented by a Directed Acrylic Graph (DAC) having a vertex for each variable Xᵢ and directed edges between vertices representing dependences between variables in the local conditional distributions Pᵢ(Xᵢ|Yⱼ₁, . . . , Y_{jK}_{ᵢ}). Subgraphs of a DAG, each including a vertex and associated directed edges are network fragments.

A model is evolved or optimized by determining the most likely factorization and the most likely parameters given the input data. This may be described as “learning a Bayesian network,” or, in other words, given a training set of input data, finding a network that best matches the input data. This is accomplished by using a scoring function that evaluates each network with respect to the input data.

A Bayesian framework is used to determine the likelihood of a factorization given the input data. Bayes Law states that the posterior probability, P(D|M), of a model M, given data D is proportional to the product of the product of the posterior probability of the data given the model assumptions, P(D|M), multiplied by the prior probability of the model, P(M), assuming that the probability of the data, P(D), is constant across models. This is expressed in the following equation:

$$P ( M \vert D ) = \frac{P ( D \vert M ) * P ( M )}{P ( D )} .$$

The posterior probability of the data assuming the model is the integral of the data likelihood over the prior distribution of parameters:

P(D|M)=∫P(D|M(Θ))P(Θ|M)dΘ.

Assuming all models are equally likely (i.e., that P(M) is a constant), the posterior probability of model M given the data D may be factored into the product of integrals over parameters for each local network fragment M, as follows:

$$P ( M \vert D ) = {\prod }_{i = 1}^{n} \int {P}_{i} ( {X}_{i} \vert {Y}_{j 1} , … , {Y}_{{jK}_{i}} ; {Θ}_{i} ) .$$

Note that in the equation above, a leading constant term has been omitted. In some embodiments, a Bayesian Information Criterion (BIC), which takes a negative logarithm of the posterior probability of the model P(D|M) may be used to “Score” each model as follows:

$${S}_{tot} ( M ) = - log P ( M \vert D ) = {\sum }_{i = 1}^{n} S ( {M}_{i} ) ,$$

where the total score Sₜₒₜ for a model M is a sum of the local scores Sᵢ for each local network fragment. The BIC further gives an expression for determining a score each individual network fragment:

$$S ( {M}_{i} ) \approx {S}_{BIC} ( {M}_{i} ) = {S}_{MLE} ( {M}_{i} ) + \frac{κ ( {M}_{i} )}{2} log N$$

where κ(Mᵢ) is the number of fitting parameter in model Mᵢ and N is the number of samples (data points). S_{MLE}(Mᵢ) is the negative logarithm of the likelihood function for a network fragment, which may be calculated from the functional relationships used for each network fragment. For a BIC score, the lower the score, the more likely a model fits the input data.

The ensemble of trial networks is globally optimized, which may be described as optimizing or evolving the networks. For example, in some embodiments, the trial networks may be evolved and optimized according to a Metropolis Monte Carlo Sampling algorithm. Simulated annealing may be used to optimize or evolve each trial network in the ensemble through local transformations. In an example simulated annealing processes, each trial network is changed by adding a network fragment from the library, by deleted a network fragment from the trial network, by substituting a network fragment or by otherwise changing network topology, and then a new score for the network is calculated. Generally speaking, if the score improves, the change is kept and if the score worsens the change is rejected. A “temperature” parameter allows some local changes which worsen the score to be kept, which aids the optimization process in avoiding some local minima The “temperature” parameter is decreased over time to allow the optimization/evolution process to converge.

All or part of the network inference process may be conducted in parallel for the trial different networks. Each network may be optimized in parallel on a separate processor and/or on a separate computing device. In some embodiments, the optimization process may be conducted on a supercomputer incorporating hundreds to thousands of processors which operate in parallel. Information may be shared among the optimization processes conducted on parallel processors.

The optimization process may include a network filter that drops any networks from the ensemble that fail to meet a threshold standard for overall score. The dropped network may be replaced by a new initial network. Further any networks that are not “scale free” may be dropped from the ensemble. After the ensemble of networks has been optimized or evolved, the result may be termed an ensemble of generated networks, which may be collectively referred to as the generated consensus network.

Simulation to Extract Quantitative Relationship Information and for Prediction

The ensemble of generated networks may be used to simulate the behavior of the biological system. Quantitative parameters of relationships in the generated networks may be extracted by applying simulated perturbations to each node individually while observing the effects on the other nodes in the generated networks. For example, the simulation for quantitative information extraction may involve perturbing (increasing or decreasing) each node in the network by 10 fold and calculating the posterior distributions for the other nodes (e.g., proteins) in the models. The endpoints are compared by t-test with the assumption of 100 samples per group and the 0.01 significance cut-off. The t-test statistic is the median of 100 t-tests. Through use of this simulation technique, an AUC (area under the curve) representing the strength of prediction and fold change representing the in silico magnitude of a node driving an end point are generated for each relationship in the ensemble of networks.

A relationship quantification module of a local computer system may be employed to direct the AI-based system to perform the perturbations and to extract the AUC information and fold information. The extracted quantitative information may include fold change and AUC for each edge connecting a parent note to a child node. In some embodiments, a custom-built R program may be used to extract the quantitative information.

In some embodiments, the ensemble of generated cell model networks can be used through simulation to predict outcomes.

The output of the AI-based system may be quantitative relationship parameters and/or other simulation predictions.

Resulting Bayesian Causal Relationship Networks

The resulting ensemble of generated networks with or without quantitative relationship information obtained from simulation may be termed a Bayesian causal relationship network representing the sliced data set. This network includes nodes representing variables for the sliced data set and directional edges representing relationships among the variables.

The network connections between the nodes representing data for different variables in the sliced data set are “probabilistic,” partly because the connection may be based on correlations between the observed data sets “learned” by the computer algorithm. For example, if the expression level of protein X and that of protein Y are positively or negatively correlated, based on statistical analysis of the data set, a causal relationship may be assigned to establish a network connection between proteins X and Y. The reliability of such a putative causal relationship may be further defined by a likelihood of the connection, which can be measured by p-value (e.g., p<0.1, 0.05, 0.01, etc.).

The network connections between the nodes representing data for different variables in the sliced data set are “directional” or “causal” partly because the network connections, as determined by the reverse-engineering process, reflect the cause and effect of the relationship between the connected variables, such that raising the expression level of variable may cause the expression level of the other to rise or fall, depending on whether the connection is stimulatory or inhibitory.

The network connections between the nodes representing data for different variables in the sliced data are “quantitative,” partly because the network connections, as determined by the process, may be simulated in silico, based on the existing data set and the probabilistic measures associated therewith. For example, in the established network connections, it may be possible to theoretically increase or decrease (e.g., by 1, 2, 3, 5, 10, 20, 30, 50,100-fold or more) the expression level of a given protein (or a “node” in the network), and quantitatively simulate its effects on other connected proteins in the network.

The network connections between the nodes representing data for different variables in the sliced data are “unbiased,” at least partly because no data points are statistically or artificially cut-off, and partly because the network connections are based on input data alone, without referring to pre-existing knowledge about the biological process in question.

The network connections between the molecular measurements in the data are “systemic” and (unbiased), partly because a broad range of potential connections among all input variables have been systemically explored un an unbiased fashion. The reliance on computing power to execute such systemic probing exponentially increases as the number of input variables increases.

In general, an ensemble of ˜500-1,000 networks is usually sufficient to predict probabilistic causal quantitative relationships among all of the variables in the sliced data set. The ensemble of networks captures uncertainty in the data and enables the calculation of confidence metrics for each model prediction. Predictions generated using the ensemble of networks together, where differences in the predictions from individual networks in the ensemble represent the degree of uncertainty in the prediction. This feature enables the assignment of confidence metrics for predictions of clinical outcome based on the networks.

Once the models are reverse-engineered, further simulation queries may be conducted on the ensemble of models to determine potential biomarkers for a clinical outcome of interest.

Generation of Differential (Delta) Networks

A differential network creation module may be used to generate differential (delta) networks between Bayesian causal relationship networks for different sliced data sets. The differential network compares all of the quantitative parameters of the relationships in the Bayesian causal relationship networks for different sliced data sets. The quantitative parameters for each relationship in the differential network are based on the comparison. In some embodiments, a differential may be performed between various differential networks, which may be termed a delta-delta network.

Such a differential networks highlights how relationships are changed in one sliced data set as compared with another sliced data set. For example, a differential network between Bayesian causal relationship networks based on sliced data for responsive patients (e.g. that exhibited an overall clinical benefit) and based on sliced data for refractory patients (e.g. that exhibited no clinical benefit) can be used to highlight differences in relationships between variables in the two patient groups.

Visualization of Networks

The relationship values for the ensemble of networks and for the differential networks may be visualized using a network visualization program (e.g., Cytoscape open source platform for complex network analysis and visualization from the Cytoscape consortium). In the visual depictions of the networks, the thickness of each edge (e.g., each line connecting the proteins) represents the strength of fold change. The edges are also directional indicating causality, and each edge has an associated prediction confidence level.

Output of CTAW

The results from the statistical analysis of the clinical trial are stored as various files. In some embodiments, the stored files includes results that are the complete outputs of regression analysis that identifies molecular correlates of time on trial and administration of agent within each enrolled patient. The regression procedure is undertaken as follows. First, the available omics data for all patient samples is determined. Next, regression analysis is performed within each patient. Following regression analysis, significant results are identified and compiled into spreadsheets. In some embodiments, in addition to spreadsheets, the significant results are visualized as heatmaps.

In some embodiments, word clouds are generated to visualize the frequency of pathway members identified by proteomics regression analysis. This approach first considers a pathway to be a set of proteins performing a biological function. Pathway membership is taken from publically available databases such as BioCarta and KEGG. Given this prior knowledge of pathway membership, the occurrence of pathway proteins in regression hits from clinical trial patients is computed. Word clouds represent this information in visual form by showing the pathway proteins found most frequently in the largest text; whereas, pathway proteins found infrequently are shown in smaller text. The directionality of proteomics regression hits is indicated on the word clouds by using color. Regression hits that are consistently up-regulated in patient samples are shown in red, while down-regulated proteins are indicated in green. Any regression hit that is up-regulated in patients as often as down-regulated is shown in black.

In some embodiments, patient reports are generated automatically following completion of the statistical analysis pipeline. The patient report may describe the methodology used in the analysis, the available omic data, and the up-regulated and down-regulated omic hits. In addition, heatmap and pathway map visualizations may be included in the patient reports in some embodiments.

Output AI-Networks

In some embodiments, one output from the CTAW 400 is a set of artificial intelligence (AI) networks generated by Bayesian Learning. AI networks, which are generated for each data slice that has been created, reveal the cause-and-effect relationships between clinical and molecular variables. For example, in the case of severe adverse events, two data slices are made: (1) data in which patients experienced adverse events of toxicity grade three and (2) data in which patients did not experience adverse events of toxicity grade three. By applying Bayesian learning, networks are learned to represent the patient data from toxicity grade three or higher adverse events, and the patient data without these severe adverse events.

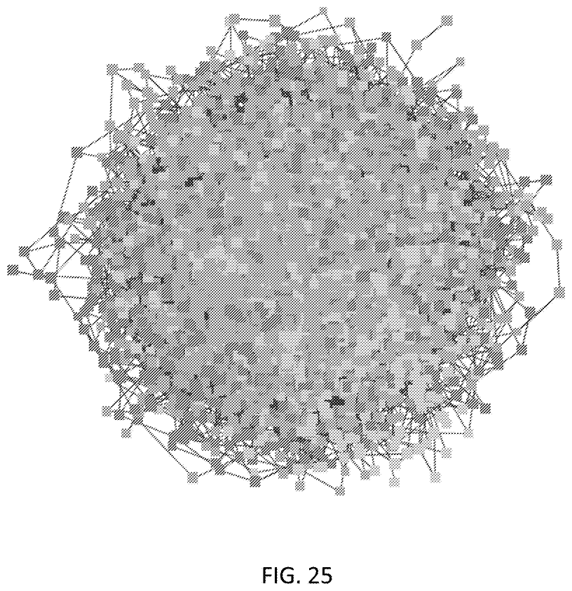

FIG. 25 illustrates an AI network that is an ensemble of networks representing data collected from patients while they had been experiencing severe adverse events related to blood and lymphatic system disorders. Severe adverse events are defined as having toxicity grade three. Any network edge with frequency less than 40% in the ensemble was removed prior to network visualization.

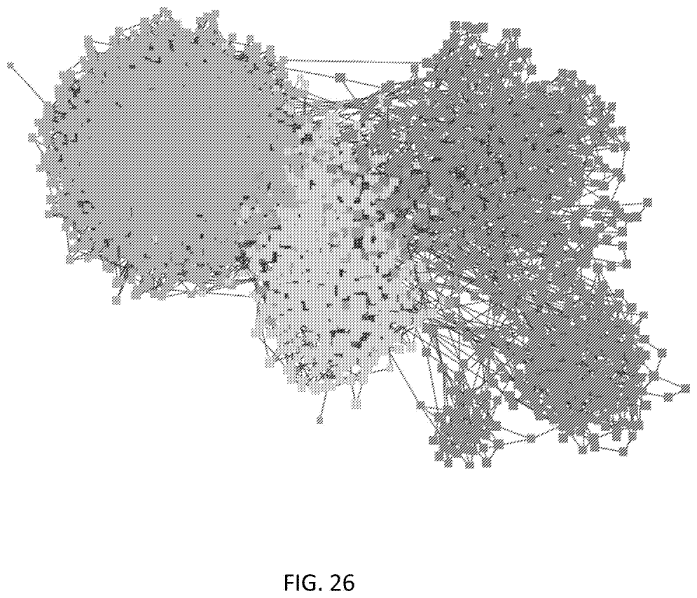

FIG. 26 illustrates an AI network that is an ensemble of networks representing data collected from patients while they had not been experiencing severe adverse events related to blood and lymphatic system disorders. As before, severe adverse events are defined as having toxicity grade three. Any network edge with frequency less than 40% in the ensemble of networks was removed prior to network visualization.

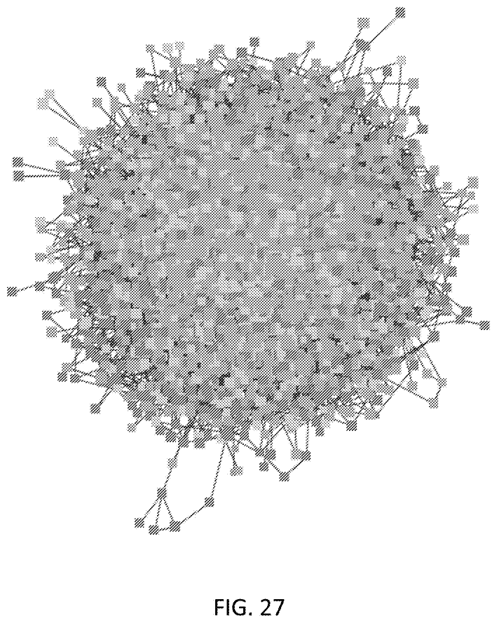

In addition to the networks learned from individual data slices, networks may be combined to gain further insight into the topological differences between phenotypic states. For instance, delta networks may be generated from a pair of two networks. Delta networks are networks composed of edges present in one network but absent from the other network, or that have a significantly different parameter in one network as opposed to the other network. For the pair of adverse events networks described above with respect to FIGS. 25 and 26, a delta network may be generated that would contain edges present in the network representing adverse events of toxicity grade three, and absent in the network representing lack of adverse events of toxicity grade three. FIG. 27 illustrates the delta network created from the pair of networks arising from the presence or absence of severe adverse events related to blood and lymphatic systems disorders. This network is limited to the edges that are present in the adverse event network and that are not present in the network learned from data in which patients had not experienced severe adverse events.

Logs

In some embodiments, as the CTAW 400 is executed, log files are generated automatically. As the workflow is running, log files allow users to monitor its progress. By checking log files, users gain confidence that data processing and later steps are proceeding in a timely fashion without encountering any unexpected input that would have caused the workflow execution to halt. In addition, monitoring log files allows the user to estimate how much time remains until the workflow execution has completed. The log files also provide records documenting actions taken during the execution of the CTAW 400. Documentation allows for users to audit retrospectively the reliability of the results generated by the CTAW.

Patient Dashboard

In some embodiments, a patient dashboard, which provides an intuitive visualization of clinical data, is output from the CTAW. FIG. 28 shows an exemplary patient dashboard. Along with demographic information, the patient dashboard provides static information regarding the initial tumor location, trial arm assigned, prior therapies, length of time enrolled, and disposition event. Clinical information that is collected throughout trial enrollment is plotted longitudinally. Examples of dynamic clinical information included in plot are tumor size, tumor response, lab measurements, and presence of adverse events. Additionally, agent infusions and cycle start dates are indicated on the patient profile. In an example embodiment, patients are plotted in the patient dashboard in order of current tumor size, such that the patients with the largest reduction in tumor size are plotted first.

Sample Map

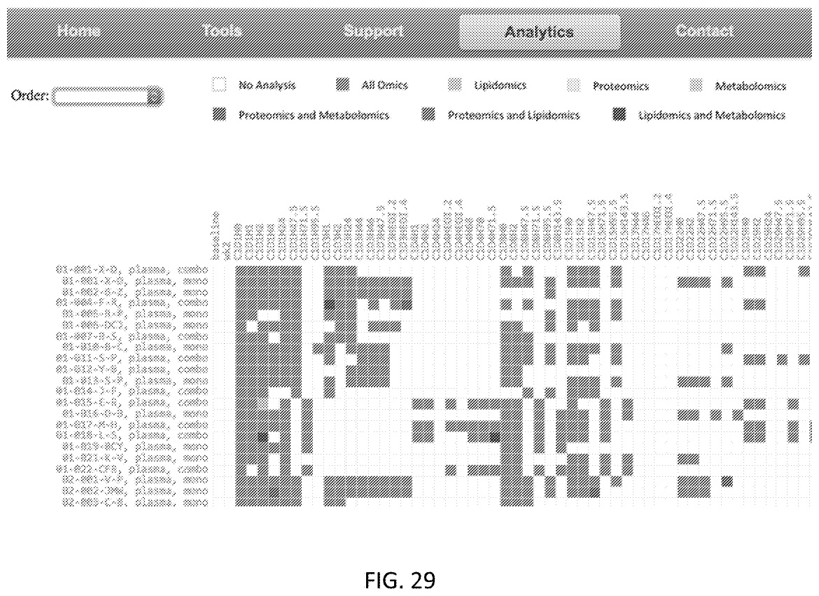

In some embodiments, a sample map, which enables interactive visualization sample data, is output from the CTAW. FIG. 29 shows an exemplary sample map. This visualization shows the available omics data for each patient sample in an interactive grid. As described above, in some embodiments, each patient has plasma, buffy coat, urine, and tissue samples collected throughout their trial enrollment. In this visualization, patient samples are represented by rows, whereas time points are represented as columns. The availability of omics data is indicated by color, with eight color levels representing the presence or absence of three omics technologies: lipidomics, proteomics, and metabolomics.

The sample map allows the user to interact with the visualized data in the following manner. Data rows may be reordered according to sample type, patient, or other criteria. Ordering by sample type shows the buffy coat samples at the top, followed by plasma, tissue, and urine. Ordering by patient lists all samples for the first patient, followed by all samples for the second patient, and so forth until the last patient. The sample map also allows for the visualization to be ordered by a particular row (patient sample) and column (time point).

Patient Map

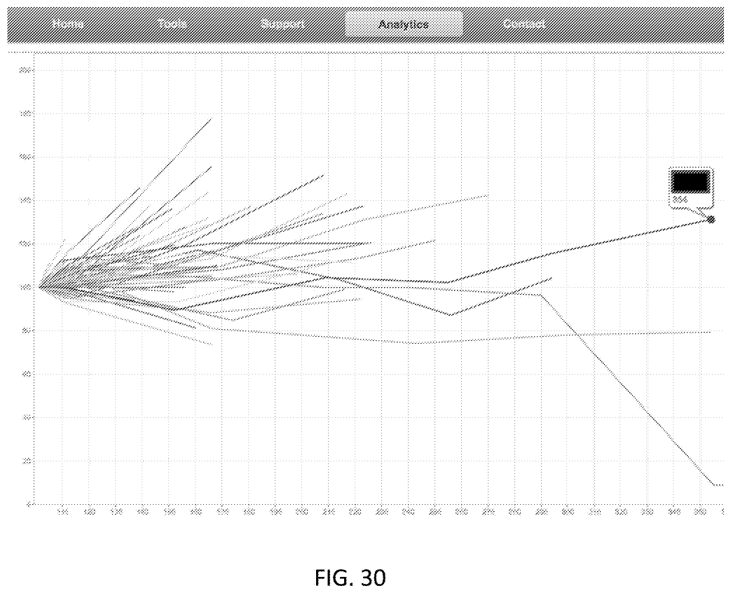

In an example embodiment, a patient map webpage provides an interactive visualization of tumor measurements made for all patients enrolled in the clinical trial. FIG. 30 shows an exemplary patient map webpage. This visualization is generated automatically as part of the CTAW. Interacting with the patient map webpage allows users to view the tumor growth of patient subsets of interest.

To be included in this patient map webpage, a patient must have had at least one tumor measurement made prior to trial start and at least one tumor measurement made following trial start. Tumor sizes are taken to be the geometric averages across tumor sites. Patient trial arm and demographic information is taken from the clinical records. Any patient with undefined treatment arm is omitted from this visualization. Patients who lack race information are given placeholder values of “Not specified.”

Users may interact with the patient map by selecting a color scheme used to color the patient tumor responses. The option to color by “Treatment,” or “Study Arm” allows the user to see which patients were assigned to the monotherapy treatment arm, or specific chemotherapeutic agents used in the combination treatment arm. Additionally, line colors may indicate patients' sex, race, age, or ethnicity. Selecting “Outcome” results in the lines being colored by the reasons for patients leaving the trial.

Determination of Potential Biomarkers (e.g., Companion Diagnostics)

As described above, in some embodiments, determination of potential biomarkers (e.g., companion diagnostic markers CDx) includes some or all of analysis of AI-networks (e.g., Bayesian networks) to identify outcome drivers, statistical analysis to identify differential expressed variables, and machine learning. As noted above, in some embodiments this includes the steps of (1) harvest variables that are drivers of key outputs related to the prediction objective in the relevant AI networks; (2) identify differentially expressed variables between the patient stratification groups at the specified time point; and (3) input the results from steps (1) and (2) into machine learning algorithm that determines which features robustly predict phenotypic outcome.

Identification of Outcome Drivers from AI Networks (e.g., Bayesian Networks)

As described in previous sections, CDx markers may be used to stratify patients on the basis of clinical response, presence of adverse events, or other criteria. One method for selecting candidate CDx markers is by finding outcome drivers. An outcome drivers is defined as a node that has a high probability of driving clinical outcome, as inferred by the AI networks. In an example embodiment, determining outcome drivers is done specifically for the desired patient stratification, and requires three specifications to be made.

The first specification is the set of clinical outcome variables related to the stratification of interest. For instance, stratifying patients in terms of clinical response may lead to a choice of clinical outcome variables to be the tumor size, tumor response, and relative tumor size. If the stratification were made according to the presence or absence of adverse events, clinical outcome variables would include appropriate adverse event variables.

The second specification is the set of AI networks from which outcome drivers should be harvested. A CDx panel with the objective of predicting patient outcome by measuring features prior to administration of an agent may consider outcome drivers derived from AI networks from individual patients during a first treatment cycle (e.g., Cycle 1).

The final specification is the type of connections to be made between outcome drivers and clinical outcome variables. Connection types include their degree and their directionality. Direct connections, which are first-degree neighbors, imply a direct causal correlation between outcome drivers and clinical outcome variables. Second-degree or higher connections include additional variables that connect indirectly. Directionality specifies if a user requires outcome drivers to influence clinical outcome variables in terms of parent to child nodes, or if the user also allows for outcome drivers to be influenced by clinical outcome variables in the reverse manner.

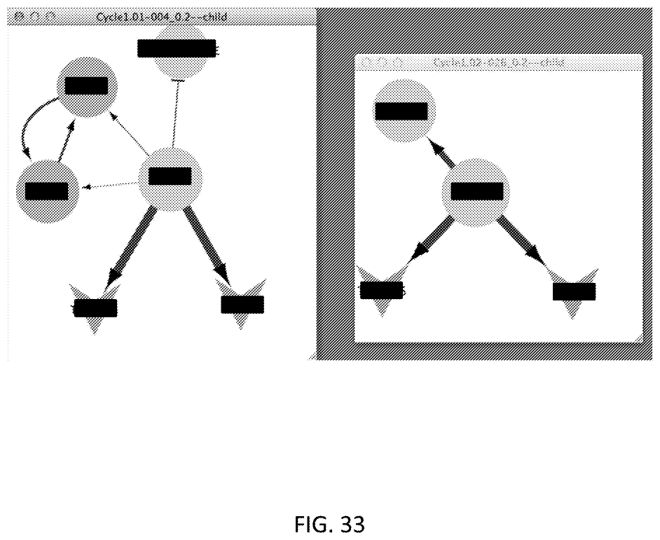

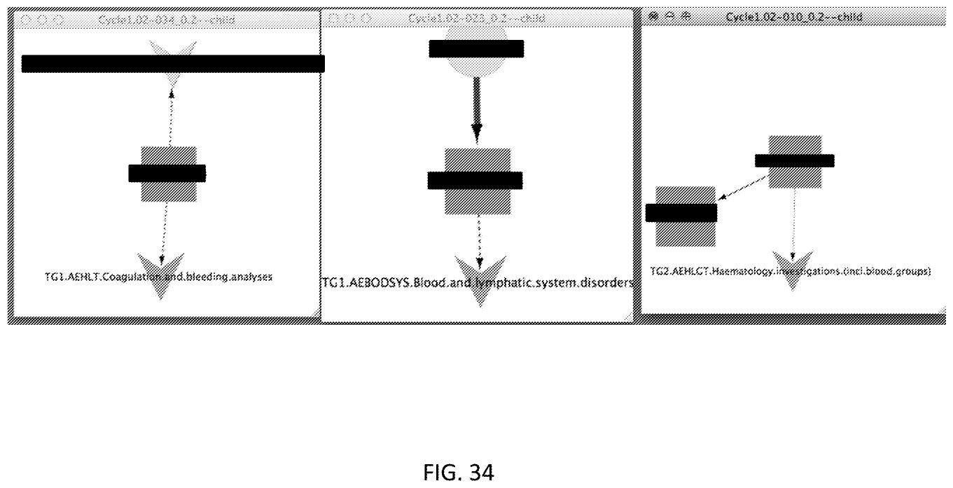

The procedure for determining outcome drivers is illustrated by two case studies: (1) stratifying patients by their response to therapy, and (2) stratifying patients based on the presence of severe adverse events. For the first case study to predict CDx markers related to patient response, 68 outcome drivers are found that serve as first-order parent nodes to clinical outcome variables in at least one of the 32 AI networks representing patient data collected during Cycle 1, as shown in FIG. 33. For the second case study to predict patient adverse events, 115 outcome drivers are found that serve as first-order parent nodes to adverse event related outcome variables, as shown in FIG. 34. In both case studies, the set of networks from which to harvest outcome drivers in the 32 AI networks representing patient data collected during Cycle 1.

Identification of Differentially Expressed Variables

In some embodiments regression analysis is employed to find omics features (proteins, lipids, and metabolites) whose abundances change in response to an agent administered during the clinical trial. The regression analysis is implemented as part of the CTAW in three main steps: (1) housekeeping, (2) statistical modeling, and (3) summarizing results.

In some embodiments, prior to beginning regression analysis, housekeeping steps are taken to archive previous results and create empty results directories. To map appropriate data sets for regression, samples in omics data are linked with annotations in the updated master file. Regression analysis is then undertaken for each combination of patient, sample type, and treatment regimen. For example, for a study with two different treatment regimens and a patient who started on one treatment regimen and then crossed over to another treatment regimen, a regression is performed using the data from when the patient was on the first regimen and another is performed regression is performed using the data from when the patient was on the second regimen Each of these regressions is further divided based on the availability of omics data sets.

Regression analysis can be based on multiple different models for a given data set. For example, a given data set may be the plasma metabolomics samples measured for patient 01-001 during a particular regimen (e.g., monotherapy). The first two models consider available samples collected during Cycle 1. Model one is a regression that relates the omics features to the fixed terms week, and hour within week. Model two is limited to week one and thus relates the omics features to the fixed term hour. The third model is a regression on pre-dose samples, and relates omic features to the fixed terms cycle and day (e.g., either Day 1 or Day 15). The fourth model is a regression on end cycle samples (e.g., Day 22 Hour 95.5) and relates omic features to the fixed term cycle. The fifth regression uses all available data to compare the effect of infusion on omic features. Finally, the sixth regression is used only for tissue samples to compare week two to baseline levels of omic features.

Following regression modeling, analysis results are summarized for individual patients. This sums the occurrences of significant features to be included in statistical analysis reports for each patient (statistical analysis reports section). In addition, arm specific summaries are generated for significant features. Finally, pathway analysis is applied to significant features using pathway membership information from KEGG, BioCarta, Reactome, and NCI.

An additional regression is performed to test hour and dose using all patient samples. This regression uses a mixed model within hour and dose considered as fixed effects and patient as a random effect.

An additional method for selecting candidate CDx markers (possible biomarkers) is to identify statistically significant omic variables or lab tests. Statistically significant features are defined as those that are either differentially expressed in the desired patient stratification or have been identified previously by regression analysis. Identifying statistically significant features as potential CDx markers requires two specifications to be made. The first specification is which statistical analysis methodology to utilize. The classic statistical analysis approach to identify differentially expressed markers between the two patient stratifications is to perform a two-sample t-test. Alternatively, limma, a methodology established by the bioinformatics community, may be used for differential expression analysis instead. The previous results from regression analysis may be mined to find statistically significant features for candidate CDx markers. This approach considers any regression hit to be statistically significant; therefore, all regression hits are evaluated as candidate CDx markers.

In an example embodiment, the second specification required to identify statistically significant candidate CDx markers is how to define statistical significance. In instances where the differential expression methodology is utilized, significance may be defined in terms of a p-value or false discovery rate (FDR) cutoff, such that any feature with p-value or FDR below the cutoff is considered significant. Common cutoffs for significant p-value and FDR are 0.05 and 0.1, respectively. Alternatively, features may be ranked by p-values so that the most significant features may be considered significant. This approach may be used to define the Top 100 features as significant without requiring the actual significance to be below a specific cutoff. If regression hits are mined as potential CDx markers, statistical significance may also be defined according to FDR values in terms of a specific cutoff or ranked list. Additional requirements on regression hits may be imposed such as requiring a regression hit to be present in the regression results from a majority of patients rather than an individual patient.

Machine Learning

In some embodiments, Prospective CDx markers, which are potential biomarkers, may be identified through the application of a machine learning approach. In some embodiments, outcome drivers identified using AI-networks and differentially expressed variables identified using statistical methods form a set of possible biomarkers, and machine learning is used to select a subset of the possible biomarkers as potential biomarkers or prospective CDx markers selecting for possible biomarkers that are predictive of the output, but that are relatively uncorrelated with the other possible biomarkers. Given that the number of molecular features and lab tests is typically much greater than the number of patients, an appropriate machine learning approach for predicting patient stratifications, in an example embodiment, is logistic regression with the elastic net penalty. Logistic regression is often plagued with degeneracies when the number of predictors p is larger than the number of variables n and exhibits unstable behavior even when n is close to p. The elastic-net penalty alleviates these issues, and regularizes and selects variables as well.

The elastic net is a shrinkage, regularization, and variable selection method. The elastic net is used to identify the set of CDx markers by simultaneously performing automatic variable selection and continuous shrinkage, and selecting groups of correlated variables. The elastic net produces a sparse elastic net model with good prediction accuracy, and further encourages a grouping effect where strongly correlated predictors (i.e., the CDx markers) tend to be in or out of the model together. The elastic net is particularly useful when the number of predictors (p) is much bigger than the number of observations (n), such as here where the number of molecular features and lab tests is typically much greater than the number of patients.

The system adapts a categorical modeling approach that utilizes an elastic net regression analysis for continuous measurements. The elastic net penalty is described by the following equation: (1−α)|β|₁+α|β|². The elastic net parameters α and λ are determined by leave-one-out cross-validation with the objective of minimizing the deviance penalty. The values of a to search are specified as 0.05 to 0.95 in increments of 0.01. The sequence of λ values to search is specified automatically by the glmnet function. Glmnet is a package implemented in the R programming system. Glmnet includes fast algorithms for estimation of generalized linear models with lasso, ridge regression, and mixtures of the two penalties (the elastic net) using cyclical coordinate descent, computed along a regularization path. In the event that more than one set of elastic net parameters yields the same cross-validation penalty (that is, the minimum deviance is tied), the maximum value of λ is selected, and the α value corresponding to this λ value is chosen.

Given the optimal elastic net parameters, bootstrap resampling is utilized to evaluate the robustness of candidate biomarkers. This process involves resampling the input data set with replacement and retraining the elastic net model, using the optimal α and λ values. By performing this bootstrap resampling 500 times, the robustness of each input feature as a predictor may be assessed by counting how often the model fit by resampled data sets includes a non-zero value in the model coefficient (β). The most robust features are those that are present in the majority of models fit by resampled data sets. Currently, this robustness cutoff is set such that any input feature that occurs in any model trained by a resampled data set is considered robust.

Applicability to Various Diseases and Disorders

The methods described in Examples 1 and 2 below for identifying candidate biomarkers in patients afflicted with solid tumors may also be applied to patients afflicted with other disorders, including but not limited to infectious diseases, autoimmune diseases (e.g. multiple sclerosis and lupus erythematosus), neuro-degenerative disorders (e.g. Alzheimer's disease and Parkinson's disease), alopecia, inflammation, diabetes (e.g. Type I and II diabetes, gestational diabetes), pre-diabetes, metabolic syndrome, and cardiovascular disease (e.g. coronary heart disease (CHD), stroke, carotid artery disease, and peripheral vascular disease (PVD)).

Although the analytical methods for identifying the candidate biomarkers in cancer patients described in Examples 1 and 2 would also generally be applicable to other disorders, the clinical data collected from each patient may vary depending on the disorder. For example, to identify candidate biomarkers for diabetes, clinical data collected from the patients may include blood glucose (e.g. fasting blood glucose, fed blood glucose), glucose tolerance, blood glucagon, insulin, insulin sensitivity, hemoglobin A1c (HbA1c) levels, body weight, waist circumference, high density lipoprotein (HDL) cholesterol, low density lipoprotein (LDL) cholesterol, total cholesterol, triglycerides, blood pressure, frequency of urination, and use of blood glucose lowering medications. Methods for clinical evaluation of patients afflicted with diabetes are known in the art and are described, for example, in US 2016/0058769 and US 2015/0359861, which are incorporated by reference herein in their entirety.

To identify candidate biomarkers for cardiovascular disease, clinical data collected from the patients may include HDL cholesterol, LDL cholesterol, total cholesterol, lipoprotein a, apolipoprotein (apo A-I), triglycerides, blood pressure, body weight, waist circumference, electrocardiogram (EKG or ECG), cardiac stress test, smoking history, history of diabetes, and use of blood pressure, blood glucose, and cholesterol lowering medications. Methods for clinical evaluation of patients afflicted with cardiovascular disease are known in the art and are described, for example, in US 2016/0139160, which is incorporated by reference herein in its entirety.

In certain embodiments, the methods described herein are used for identifying potential biomarkers that are predictive of a patient's response to a therapeutic agent for a particular disorder. For example, in some embodiments the candidate biomarkers may be used to predict the efficacy of a therapeutic agent in treating the disorder, or the likelihood of an adverse event in response to the therapeutic agent.

In certain embodiments, the disorder is diabetes (e.g., Type I diabetes, Type II diabetes, or gestational diabetes). Suitable therapeutic agents for diabetes include, but are not limited to a meglitinide, a sulfonylurea, a dipeptidy peptidase-4 (DPP-4) inhibitor, a biguanide, a thiazolidinediones, an alpha-glucosidase inhibitor, an amylin mimetic; an incretin mimetics; an insulin; and any combination thereof. In a particular embodiment, the therapeutic agent for the treatment of diabetes is an HSP90 inhibitor, for example, an HSP90β inhibitor. In another embodiment, the therapeutic agent is for the treatment of diabetes is EN01 or an EN01 containing molecule.

In certain embodiments, the disorder is cardiovascular disease. Suitable therapeutic agents for cardiovascular disease include, but are not limited to statins (HMG-CoA reductase inhibitors), antihypertensive agents, thrombolytic agents, and anti-platelet and anticoagulation therapies. Statins include, for example, atorvastatin, fluvastatin, lovastatin, pitavastatin, pravastatin, rosuvastatin and simvastatin. Antihypertensive agents include, for example, angiotensin-converting enzyme (ACE) inhibitors, blockers of the adrenergic nervous system (beta and alpha adrenergic blockers), calcium-channel blockers, and angiotensin-receptor blockers (ARBs). Anti-platelet and anticoagulation therapies include, for example, heparin, glycoprotein IIb/IIIa inhibitors, clopidogrel, and warfarin.

In certain embodiments, the disorder is a cancer. In certain embodiments, the cancer is not a central nervous system (CNS) cancer, i.e., not a cancer of a tumor present in at least one of the spinal cord, the brain, and the eye. In certain embodiments, the primary cancer is not a CNS cancer. In certain embodiments, the cancer is a blood tumor (i.e., a non-solid tumor). In certain embodiments, the cancer comprises a solid tumor. In certain embodiments, the solid tumor is selected from the group consisting of carcinoma, melanoma, sarcoma, and lymphoma. In certain embodiments, the solid tumor is selected from the group consisting of breast cancer, bladder cancer, colon cancer, rectal cancer, endometrial cancer, kidney (renal cell) cancer, lung cancer, melanoma, pancreatic cancer, prostate cancer, thyroid cancer, skin cancer, bone cancer, brain cancer, cervical cancer, liver cancer, stomach cancer, mouth and oral cancers, neuroblastoma, testicular cancer, uterine cancer, thyroid cancer, and vulvar cancer. In certain embodiments, the skin cancer is melanoma, squamous cell carcinoma, or cutaneous T-cell lymphoma (CTCL).

Suitable therapeutic agents for the treatment of cancer include, but are not limited to, small molecule chemotherapeutic agents and biologics. In a particular embodiment, the therapeutic agent for the treatment of cancer is Coenzyme Q10.

Small molecule chemotherapeutic agents generally belong to various classes including, for example: 1. Topoisomerase II inhibitors (cytotoxic antibiotics), such as the anthracyclines/anthracenediones, e.g., doxorubicin, epirubicin, idarubicin and nemorubicin, the anthraquinones, e.g., mitoxantrone and losoxantrone, and the podophillotoxines, e.g., etoposide and teniposide; 2. Agents that affect microtubule formation (mitotic inhibitors), such as plant alkaloids (e.g., a compound belonging to a family of alkaline, nitrogen-containing molecules derived from plants that are biologically active and cytotoxic), e.g., taxanes, e.g., paclitaxel and docetaxel, and the vinka alkaloids, e.g., vinblastine, vincristine, and vinorelbine, and derivatives of podophyllotoxin; 3. Alkylating agents, such as nitrogen mustards, ethyleneimine compounds, alkyl sulphonates and other compounds with an alkylating action such as nitrosoureas, dacarbazine, cyclophosphamide, ifosfamide and melphalan; 4. Antimetabolites (nucleoside inhibitors), for example, folates, e.g., folic acid, fiuropyrimidines, purine or pyrimidine analogues such as 5-fluorouracil, capecitabine, gemcitabine, methotrexate, and edatrexate; 5. Topoisomerase I inhibitors, such as topotecan, irinotecan, and 9- nitrocamptothecin, camptothecin derivatives, and retinoic acid; and 6. Platinum compounds/complexes, such as cisplatin, oxaliplatin, and carboplatin.

Exemplary chemotherapeutic agents include, but are not limited to, amifostine (ethyol), cisplatin, dacarbazine (DTIC), dactinomycin, mechlorethamine (nitrogen mustard), streptozocin, cyclophosphamide, carrnustine (BCNU), lomustine (CCNU), doxorubicin (adriamycin), doxorubicin lipo (doxil), gemcitabine (gemzar), daunorubicin, daunorubicin lipo (daunoxome), procarbazine, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-I1, lO-hydroxy-7-ethyl-camptothecin (SN38), dacarbazine, S-I capecitabine, ftorafur, 5′deoxyflurouridine, UFT, eniluracil, deoxycytidine, 5-azacytosine, 5- azadeoxycytosine, allopurinol, 2-chloro adenosine, trimetrexate, aminopterin, methylene-10-deazaaminopterin (MDAM), oxaplatin, picoplatin, tetraplatin, satraplatin, platinum-DACH, ormaplatin, CI-973, JM-216, and analogs thereof, epirubicin, etoposide phosphate, 9- aminocamptothecin, 10,11-methylenedioxycamptothecin, karenitecin, 9-nitrocamptothecin, TAS 103, vindesine, L-phenylalanine mustard, ifosphamidemefosphamide, perfosfamide, trophosphamide carmustine, semustine, epothilones A-E, tomudex, 6-mercaptopurine, 6-thioguanine, amsacrine, etoposide phosphate, karenitecin, acyclovir, valacyclovir, ganciclovir, amantadine, rimantadine, lamivudine, zidovudine, bevacizumab, trastuzumab, rituximab, 5-Fluorouracil, Capecitabine, Pentostatin, Trimetrexate, Cladribine, floxuridine, fludarabine, hydroxyurea, ifosfamide, idarubicin, mesna, irinotecan, mitoxantrone, topotecan, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil, cisplatin, doxorubicin, paclitaxel (taxol), bleomycin, mTor, epidermal growth factor receptor (EGFR), and fibroblast growth factors (FGF) and combinations thereof which are readily apparent to one of skill in the art based on the appropriate standard of care for a particular tumor or cancer.

Biologic agents (also called biologics) are the products of a biological system, e.g., an organism, cell, or recombinant system. Examples of suitable biologic agents for the treatment of cancer include nucleic acid molecules (e.g., antisense nucleic acid molecules), interferons, interleukins, colony-stimulating factors, antibodies, e.g., monoclonal antibodies, antibody-drug conjugates, chimeric antigen receptors, anti-angiogenesis agents, and cytokines. Exemplary biologic agents generally belong to various classes including, for example: 1. Hormones, hormonal analogues, and hormonal complexes, e.g., estrogens and estrogen analogs, progesterone, progesterone analogs and progestins, androgens, adrenocorticosteroids, antiestrogens, antiandrogens, antitestosterones, adrenal steroid inhibitors, and anti-leuteinizing hormones; and 2. Enzymes, proteins, peptides, polyclonal and/or monoclonal antibodies, such as interleukins, interferons, colony stimulating factor, etc.

Predictive Methods of the Invention

The present invention is based, at least in part, on the discovery that the biomarker Protein Disulfide Isomerase Family A Member 3, also referred to herein as PDIA3, is expressed at a higher than average level in the serum of subjects that are clinically responsive to treatment of cancer with Coenzyme Q10 (CoQ10), and is expressed at a lower than average level in the serum of subjects that are refractory to the treatment of cancer with CoQ10. A determination of the expression levels of PDIA3 in a sample from a subject having cancer allows physicians to make more informed treatment decisions, and to customize the treatment of the cancer to the needs of individual subjects, thereby maximizing the benefit of treatment and minimizing the exposure of patients to unnecessary treatments which may not provide any significant benefits and often carry serious risks due to toxic side-effects.

Accordingly, the present invention provides methods for predicting the response of a subject having cancer to treatment with CoQ10, selecting a subject with cancer as a good candidate for treatment of the cancer with CoQ10, and treating a subject having cancer with CoQ10 based on the expression level of PDIA3 in a sample obtained from the subject.

In one aspect, the present invention provides methods for selecting a subject for treatment of a cancer with Coenzyme Q10 (CoQ10), comprising: (a) detecting the level of PDIA3 in a biological sample of the subject, and (b) comparing the level of PDIA3 in the biological sample with a predetermined threshold value, wherein the subject is selected for treatment of a cancer with CoQ10 if the level of PDIA3 is above the predetermined threshold value.

In another aspect, the present invention provides methods for predicting whether a subject having a cancer will be responsive or non-responsive (refractory) to treatment with Coenzyme Q10 (CoQ10), comprising: (a) detecting the level of PDIA3 in a biological sample of the subject, and (b) comparing the level of PDIA3 in the biological sample with a predetermined threshold value, wherein a level of PDIA3 above the predetermined threshold value indicates the subject is likely to respond to treatment of a cancer with CoQ10.

In another aspect, methods of treating cancer in a subject are provided, comprising: (a) obtaining a biological sample from the subject, (b) submitting the biological sample from the subject to obtain diagnostic information as to the level of PDIA3, (c) administering a therapeutically effective amount of CoQ10 to the subject if the level of PDIA3 in the biological sample is above a threshold level.

In still another aspect, methods of treating cancer in a subject are provided, comprising: (a) obtaining diagnostic information as to the level of PDIA3 in a biological sample from the subject, and (b) administering CoQ10 to the subject if the level of PDIA3 in the biological sample is above a threshold level.

In yet another aspect, the present invention provides methods of treating cancer in a subject comprising: (a) obtaining a biological sample from the subject for use in identifying diagnostic information as to the level of PDIA3, (b) measuring the level of PDIA3 in the biological sample from the subject, (c) recommending to a healthcare provider to administer CoQ10 to the subject if the level of PDIA3 is above a threshold level.

As used herein, a “threshold value” or “threshold value” of PDIA3 refers to the level of PDIA3 (e.g., the expression level or quantity (e.g., ng/ml) in a biological sample) in a corresponding control/normal sample or group of control/normal samples obtained from subjects, e.g., similarly situated subjects such as subjects having the same cancer and who have not yet been treated with CoQ10, or normal or healthy subjects, e.g., subjects that do not have cancer. The predetermined threshold value may be determined prior to or concurrently with measurement of PDIA3 levels in a biological sample. The control sample may be from the same subject at a previous time or from different subjects.

The gene and protein sequences of PDIA3 are known in the art, and can be found, for example, at UniProtKB P30101, or Entrez Gene 2923, and at the NCBI reference sequence NP_005304.3.

In some embodiments the cancer to be treated is a solid tumor. The solid tumor can be any type of solid tumor, including any type of solid tumor described herein. In certain embodiments, the cancer to be treated is selected from the group consisting of squamous cell carcinoma, glioblastoma, and pancreatic cancer.

In certain embodiments, the biological sample is selected from the group consisting of blood, serum, urine, organ tissue, biopsy tissue, feces, skin, hair, and cheek tissue.

In another embodiment, a method of determining a clinical course of therapy for treating cancer in a subject is disclosed. In certain embodiments, the method includes determining the subject's PDIA3 expression level in a biological sample obtained from the subject, and identifying a clinical course of therapy based on the subject's PDIA3 expression level. In a specific embodiment, therapy with CoQ10 is selected when the level of PDIA3 in the biological sample is above a threshold level.

In one embodiment, one or more additional anti-cancer therapeutic agents can be administered to the patient (either sequentially or concurrently), in addition to CoQ10, including, but not limited, to chemotherapy or radiation.

Tissue Samples

The present invention may be practiced with any suitable biological sample that potentially contains, expresses, includes, PDIA3, e.g., a PDIA3 polypeptide, a nucleic acid, mRNA, or microRNA. For example, the biological sample may be obtained from sources that include whole blood and serum to diseased (e.g., tumor, including tumor of the pancreas, glioblastoma, or squamous cell carcinoma) and/or healthy tissue. In one embodiment, the biological sample is selected from the group consisting of blood, serum, urine, organ tissue, biopsy tissue, feces, skin, hair, and cheek tissue. In a preferred embodiment, the biological sample is a serum sample. In another embodiment, the present invention may be practiced with any suitable tissue samples which are freshly isolated or which have been frozen or stored after having been collected from a subject, or archival tissue samples, for example, with known diagnosis, treatment and/or outcome history. Tissue may be collected by any non-invasive means, such as, for example, fine needle aspiration and needle biopsy, or alternatively, by an invasive method, including, for example, surgical biopsy.

The inventive methods may be performed at the single cell level (e.g., isolation and testing of cancerous cells). However, preferably, the inventive methods are performed using a sample comprising many cells, where the assay is “averaging” expression over the entire collection of cells and tissue present in the sample. Preferably, there is enough of the tissue sample to accurately and reliably determine the expression levels of PDIA3. In certain embodiments, multiple samples may be taken from the same tissue in order to obtain a representative sampling of the tissue. In addition, sufficient biological material can be obtained in order to perform duplicate, triplicate or further rounds of testing.

Any commercial device or system for isolating and/or obtaining tissue and/or blood or other biological products, and/or for processing said materials prior to conducting a detection reaction is contemplated.

In certain embodiments, the present invention relates to detecting PDIA3 nucleic acid molecules (e.g., mRNA encoding PDIA3). In such embodiments, RNA can be extracted from a biological sample, before analysis. Methods of RNA extraction are well known in the art (see, for example, J. Sambrook et al., “Molecular Cloning: A Laboratory Manual”, 1989, 2^{nd} Ed., Cold Spring Harbour Laboratory Press: New York). Most methods of RNA isolation from bodily fluids or tissues are based on the disruption of the tissue in the presence of protein denaturants to quickly and effectively inactivate RNases. Generally, RNA isolation reagents comprise, among other components, guanidinium thiocyanate and/or beta-mercaptoethanol, which are known to act as RNase inhibitors. Isolated total RNA is then further purified from the protein contaminants and concentrated by selective ethanol precipitations, phenol/chloroform extractions followed by isopropanol precipitation (see, for example, P. Chomczynski and N. Sacchi, Anal. Biochem., 1987, 162: 156-159) or cesium chloride, lithium chloride or cesium trifluoroacetate gradient centrifugations.

Numerous different and versatile kits can be used to extract RNA (i.e., total RNA or mRNA) from bodily fluids or tissues (e.g., prostate tissue samples) and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), GIBCO BRL (Gaithersburg, Md.), and Giagen, Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

In certain embodiments, after extraction, mRNA is amplified, and transcribed into cDNA, which can then serve as template for multiple rounds of transcription by the appropriate RNA polymerase. Amplification methods are well known in the art (see, for example, A. R. Kimmel and S. L. Berger, Methods Enzymol. 1987, 152: 307-316; J. Sambrook et al., “Molecular Cloning: A Laboratory Manual”, 1989, 2.sup.nd Ed., Cold Spring Harbour Laboratory Press: New York; “Short Protocols in Molecular Biology”, F. M. Ausubel (Ed.), 2002, 5.sup.th Ed., John Wiley & Sons; U.S. Pat. Nos. 4,683,195; 4,683,202 and 4,800,159). Reverse transcription reactions may be carried out using non-specific primers, such as an anchored oligo-dT primer, or random sequence primers, or using a target-specific primer complementary to the RNA for each genetic probe being monitored, or using thermostable DNA polymerases (such as avian myeloblastosis virus reverse transcriptase or Moloney murine leukemia virus reverse transcriptase).

In certain embodiments, the RNA isolated from the sample (for example, after amplification and/or conversion to cDNA or cRNA) is labeled with a detectable agent before being analyzed. The role of a detectable agent is to facilitate detection of RNA or to allow visualization of hybridized nucleic acid fragments (e.g., nucleic acid fragments hybridized to genetic probes in an array-based assay). Preferably, the detectable agent is selected such that it generates a signal which can be measured and whose intensity is related to the amount of labeled nucleic acids present in the sample being analyzed. In array-based analysis methods, the detectable agent is also preferably selected such that it generates a localized signal, thereby allowing spatial resolution of the signal from each spot on the array.

Methods for labeling nucleic acid molecules are well-known in the art. For a review of labeling protocols, label detection techniques and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labeling methods include: incorporation of radioactive agents, direct attachment of fluorescent dyes (see, for example, L. M. Smith et al., Nucl. Acids Res. 1985, 13: 2399-2412) or of enzymes (see, for example, B. A. Connoly and P. Rider, Nucl. Acids. Res. 1985, 13: 4485-4502); chemical modifications of nucleic acid fragments making them detectable immunochemically or by other affinity reactions (see, for example, T. R. Broker et al., Nucl. Acids Res. 1978, 5: 363-384; E. A. Bayer et al., Methods of Biochem. Analysis, 1980, 26: 1-45; R. Langer et al., Proc. Natl. Acad. Sci. USA, 1981, 78: 6633-6637; R. W. Richardson et al., Nucl. Acids Res. 1983, 11: 6167-6184; D. J. Brigati et al., Virol. 1983, 126: 32-50; P. Tchen et al., Proc. Natl Acad. Sci. USA, 1984, 81: 3466-3470; J. E. Landegent et al., Exp. Cell Res. 1984, 15: 61-72; and A. H. Hopman et al., Exp. Cell Res. 1987, 169: 357-368); and enzyme-mediated labeling methods, such as random priming, nick translation, PCR and tailing with terminal transferase (for a review on enzymatic labeling, see, for example, J. Temsamani and S. Agrawal, Mol. Biotechnol. 1996, 5: 223-232).

Any of a wide variety of detectable agents can be used in the practice of the present invention. Suitable detectable agents include, but are not limited to: various ligands, radionuclides, fluorescent dyes, chemiluminescent agents, microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens and proteins for which antisera or monoclonal antibodies are available.

However, in some embodiments, the PDIA3 expression levels are determined by detecting the expression of a PDIA3 gene product (e.g., PDIA3 protein) thereby eliminating the need to obtain a genetic sample (e.g., RNA) from the subject sample.

Archived tissue samples, which can be used for all methods of the invention, typically have been obtained from a source and preserved. Preferred methods of preservation include, but are not limited to paraffin embedding, ethanol fixation and formalin, including formaldehyde and other derivatives, fixation as are known in the art. A tissue sample may be temporally “old”, e.g. months or years old, or recently fixed. For example, post-surgical procedures generally include a fixation step on excised tissue for histological analysis. In a preferred embodiment, the tissue sample is a diseased tissue sample, e.g., a cancer tissue, including primary and secondary tumor tissues as well as lymph node tissue and metastatic tissue.

Thus, an archived sample can be heterogeneous and encompass more than one cell or tissue type, for example, tumor and non-tumor tissue. Preferred tissue samples include solid tumor samples including, but not limited to, tumors of the pancreas, glioblastoma, or squamous cell carcinoma. It is understood that in applications of the present invention to conditions other than pancreas, glioblastoma, or squamous cell carcinoma, the tumor source can be brain, bone, heart, breast, ovaries, prostate, uterus, spleen, pancreas, liver, kidneys, bladder, stomach and muscle. Similarly, depending on the condition, suitable tissue samples include, but are not limited to, bodily fluids (including, but not limited to, blood, urine, serum, lymph, saliva, anal and vaginal secretions, perspiration and semen, of virtually any organism, with mammalian samples being preferred and human samples being particularly preferred).

Detection And/Or Measurement Of Biomarkers

The present invention contemplates any suitable means, techniques, and/or procedures for detecting and/or measuring PDIA3. The skilled artisan will appreciate that the methodologies employed to measure PDIA3 will depend at least on the type of PDIA3 being detected or measured (e.g., mRNA or polypeptide) and the source of the biological sample. Certain biological sample may also require certain specialized treatments prior to measuring PDIA3, e.g., the preparation of mRNA from a biopsy tissue in the case where PDIA3 mRNA is being measured.

In one embodiment, the present invention provides methods for selecting a subject for treatment of a cancer with CoQ10, comprising: (a) contacting a biological sample with a reagent that selectively binds to PDIA3; (b) allowing a complex to form between the reagent and PDIA3; (c) detecting the level of the complex, and (d) comparing the level of the complex with a predetermined threshold value, wherein the subject is selected for treatment of a cancer with CoQ10 if the level of the complex is above the predetermined threshold value.

In another embodiment, the present invention provides methods for predicting whether a subject having a cancer will respond to treatment with CoQ10, comprising: (a) contacting a biological sample with a reagent that selectively binds to PDIA3; (b) allowing a complex to form between the reagent and PDIA3; (c) detecting the level of the complex, and (d) comparing the level of the complex with a predetermined threshold value, wherein a level of PDIA3 above the predetermined threshold value indicates the subject is likely to respond to treatment of a cancer with CoQ10.

In one embodiment, detecting the level of the complex further comprises contacting the complex with a detectable secondary antibody and measuring the level of the secondary antibody.

In one embodiment, the reagent is an anti-PDIA3 antibody that selectively binds to at least one epitope of PDIA3. In another embodiment, the PDIA3 protein in the biological sample can be determined by immunoassay or ELISA. In another embodiment, the PDIA3 protein in the biological sample can also be determined by mass spectrometry.

In another embodiment, detecting the level of PDIA3 in a biological sample of the subject comprises determining the amount of PDIA3 mRNA in the biological sample. For example, an amplification reaction is used for determining the amount of PDIA3 mRNA in the biological sample. The amplification reaction can comprise, for example, a polymerase chain reaction (PCR); a nucleic acid sequence-based amplification assay (NASBA); a transcription mediated amplification (TMA); a ligase chain reaction (LCR); or a strand displacement amplification (SDA).

In another embodiment, a hybridization assay is used for determining the amount of PDIA3 mRNA in the biological sample. For example, an oligonucleotide that is complementary to a portion of a PDIA3 mRNA can be used in the hybridization assay to detect the PDIA3 mRNA.

Various methods for determining the levels of PDIA3 protein and mRNA are described in detail below.

1. Detection Of Nucleic Acid Biomarkers

In certain embodiments, the invention involves the detection of PDIA3 nucleic acid. In various embodiments, the diagnostic/prognostic methods of the present invention generally involve the determination of expression levels of PDIA3 in a tissue sample. Determination of gene expression levels in the practice of the inventive methods may be performed by any suitable method. For example, determination of gene expression levels may be performed by detecting the expression of mRNA expressed from the genes of interest and/or by detecting the expression of a polypeptide encoded by the genes.

For detecting nucleic acids encoding PDIA3, any suitable method can be used, including, but not limited to, Southern blot analysis, Northern blot analysis, polymerase chain reaction (PCR) (see, for example, U.S. Pat. Nos. 4,683,195; 4,683,202, and 6,040,166; “PCR Protocols: A Guide to Methods and Applications”, Innis et al. (Eds), 1990, Academic Press: New York), reverse transcriptase PCR (RT-PCT), anchored PCR, competitive PCR (see, for example, U.S. Pat. No. 5,747,251), rapid amplification of cDNA ends (RACE) (see, for example, “Gene Cloning and Analysis: Current Innovations, 1997, pp. 99-115); ligase chain reaction (LCR) (see, for example, EP 01 320 308), one-sided PCR (Ohara et al., Proc. Natl. Acad. Sci., 1989, 86: 5673-5677), in situ hybridization, Taqman-based assays (Holland et al., Proc. Natl. Acad. Sci., 1991, 88: 7276-7280), differential display (see, for example, Liang et al., Nucl. Acid. Res., 1993, 21: 3269-3275) and other RNA fingerprinting techniques, nucleic acid sequence based amplification (NASBA) and other transcription based amplification systems (see, for example, U.S. Pat. Nos. 5,409,818 and 5,554,527), Qbeta Replicase, Strand Displacement Amplification (SDA), Repair Chain Reaction (RCR), nuclease protection assays, subtraction-based methods, Rapid-Scan®, etc.

In other embodiments, gene expression levels of PDIA3 may be determined by amplifying complementary DNA (cDNA) or complementary RNA (cRNA) produced from mRNA and analyzing it using a microarray. A number of different array configurations and methods of their production are known to those skilled in the art (see, for example, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637).

Nucleic acid used as a template for amplification can be isolated from cells contained in the biological sample, according to standard methodologies. (Sambrook et al., 1989) The nucleic acid may be genomic DNA or fractionated or whole cell RNA. Where RNA is used, it may be desired to convert the RNA to a complementary cDNA. In one embodiment, the RNA is whole cell RNA and is used directly as the template for amplification.

Pairs of primers that selectively hybridize to nucleic acids corresponding to a PDIA3 nucleotide sequence are contacted with the isolated nucleic acid under conditions that permit selective hybridization. Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced. Next, the amplification product is detected. In certain applications, the detection may be performed by visual means. Alternatively, the detection may involve indirect identification of the product via chemiluminescence, radioactive scintigraphy of incorporated radiolabel or fluorescent label or even via a system using electrical or thermal impulse signals (Affymax technology; Bellus, 1994). Following detection, one may compare the results seen in a given patient with a statistically significant reference group of normal patients and cancer patients. In this way, it is possible to correlate the amount of nucleic acid detected with various clinical states.

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences may be employed. Primers may be provided in double-stranded or single-stranded form, although the single-stranded form is preferred.

A number of template dependent processes are available to amplify the nucleic acid sequences present in a given template sample. One of the best known amplification methods is the polymerase chain reaction (referred to as PCR) which is described in detail in U.S. Pat. Nos. 4,683,195, 4,683,202 and 4,800,159, and in Innis et al., 1990, each of which is incorporated herein by reference in its entirety.

In PCR, two primer sequences are prepared which are complementary to regions on opposite complementary strands of the target nucleic acid sequence. An excess of deoxynucleoside triphosphates are added to a reaction mixture along with a DNA polymerase, e.g., Taq polymerase. If the target nucleic acid sequence is present in a sample, the primers will bind to the target nucleic acid and the polymerase will cause the primers to be extended along the target nucleic acid sequence by adding on nucleotides. By raising and lowering the temperature of the reaction mixture, the extended primers will dissociate from the target nucleic acid to form reaction products, excess primers will bind to the target nucleic acid and to the reaction products and the process is repeated.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641 filed Dec. 21, 1990. Polymerase chain reaction methodologies are well known in the art.

Another method for amplification is the ligase chain reaction (“LCR”), disclosed in European Application No. 320 308, incorporated herein by reference in its entirely. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, also may be used as still another amplification method in the present invention. In this method, a replicative sequence of RNA which has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′[α-thio]-triphosphates in one strand of a restriction site also may be useful in the amplification of nucleic acids in the present invention. Walker et al. (1992), incorporated herein by reference in its entirety.

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases may be added as biotinylated derivatives for easy detection. A similar approach is used in SDA. Target specific sequences also may be detected using a cyclic probe reaction (CPR). In CPR, a probe having 3′ and 5′ sequences of non-specific DNA and a middle sequence of specific RNA is hybridized to DNA which is present in a sample. Upon hybridization, the reaction is treated with RNase H, and the products of the probe identified as distinctive products which are released after digestion. The original template is annealed to another cycling probe and the reaction is repeated.

Still other amplification methods described in GB Application No. 2 202 328, and in PCT Application No. PCT/US89/01025, each of which is incorporated herein by reference in its entirety, may be used in accordance with the present invention. In the former application, “modified” primers are used in a PCR like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other contemplated nucleic acid amplification procedures include transcription-based amplification systems (TAS), including nucleic acid sequence based amplification (NASBA) and 3SR. Kwoh et al. (1989); Gingeras et al., PCT Application WO 88/10315, incorporated herein by reference in their entirety.

Davey et al., European Application No. 329 822 (incorporated herein by reference in its entirely) disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase 1), resulting in a double-stranded DNA (“dsDNA”) molecule, having a sequence identical to that of the original RNA between the primers and having additionally, at one end, a promoter sequence. This promoter sequence may be used by the appropriate RNA polymerase to make many RNA copies of the DNA. These copies may then re-enter the cycle leading to very swift amplification. With proper choice of enzymes, this amplification may be done isothermally without addition of enzymes at each cycle. Because of the cyclical nature of this process, the starting sequence may be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR.™.” Frohman (1990) and Ohara et al. (1989), each herein incorporated by reference in their entirety.

Methods based on ligation of two (or more) oligonucleotides in the presence of nucleic acid having the sequence of the resulting “di-oligonucleotide”, thereby amplifying the di-oligonucleotide, also may be used in the amplification step of the present invention. Wu et al. (1989), incorporated herein by reference in its entirety.

Oligonucleotide probes or primers of the present invention may be of any suitable length, depending on the particular assay format and the particular needs and targeted sequences employed. In a preferred embodiment, the oligonucleotide probes or primers are at least 10 nucleotides in length (preferably, 10, 11, 12, 13, 14, 15, 16, 17, 18, 19, 20, 21, 22, 23, 24, 25, 26, 27, 28, 29, 30, 31, 32 . . . ) and they may be adapted to be especially suited for a chosen nucleic acid amplification system and/or hybridization system used. Longer probes and primers are also within the scope of the present invention as well known in the art. Primers having more than 30, more than 40, more than 50 nucleotides and probes having more than 100, more than 200, more than 300, more than 500 more than 800 and more than 1000 nucleotides in length are also covered by the present invention. Of course, longer primers have the disadvantage of being more expensive and thus, primers having between 12 and 30 nucleotides in length are usually designed and used in the art. As well known in the art, probes ranging from 10 to more than 2000 nucleotides in length can be used in the methods of the present invention. As for the % of identity described above, non-specifically described sizes of probes and primers (e.g., 16, 17, 31, 24, 39, 350, 450, 550, 900, 1240 nucleotides, . . . ) are also within the scope of the present invention. In one embodiment, the oligonucleotide probes or primers of the present invention specifically hybridize with a PDIA3 RNA (or its complementary sequence) or a PDIA3 mRNA.

In other embodiments, the detection means can utilize a hybridization technique, e.g., where a specific primer or probe is selected to anneal to a target biomarker of interest, e.g., PDIA3, and thereafter detection of selective hybridization is made. As commonly known in the art, the oligonucleotide probes and primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1994, in Current Protocols in Molecular Biology, John Wiley & Sons Inc., N.Y.).

To enable hybridization to occur under the assay conditions of the present invention, oligonucleotide primers and probes should comprise an oligonucleotide sequence that has at least 70% (at least 71%, 72%, 73%, 74%), preferably at least 75% (75%, 76%, 77%, 78%, 79%, 80%, 81%, 82%, 83%, 84%, 85%, 86%, 87%, 88%, 89%) and more preferably at least 90% (90%, 91%, 92%, 93%, 94%, 95%, 96%, 97%, 98%, 99%, 100%) identity to a portion of a PDIA3 or polynucleotide of another biomarker of the invention. Probes and primers of the present invention are those that hybridize under stringent hybridization conditions and those that hybridize to biomarker homologs of the invention under at least moderately stringent conditions. In certain embodiments probes and primers of the present invention have complete sequence identity to the biomarkers of the invention (PDIA3, gene sequences (e.g., cDNA or mRNA). It should be understood that other probes and primers could be easily designed and used in the present invention based on the biomarkers of the invention disclosed herein by using methods of computer alignment and sequence analysis known in the art (cf. Molecular Cloning: A Laboratory Manual, Third Edition, edited by Cold Spring Harbor Laboratory, 2000).

2. Detection of Polypeptide Biomarkers

The present invention contemplates any suitable method for detecting PDIA3 polypeptide. In certain embodiments, the detection method is an immunodetection method involving an antibody that specifically binds to PDIA3. The steps of various useful immunodetection methods have been described in the scientific literature, such as, e.g., Nakamura et al. (1987), which is incorporated herein by reference.

In general, the immunobinding methods include obtaining a sample suspected of containing a biomarker protein, peptide or antibody, and contacting the sample with an antibody or protein or peptide in accordance with the present invention, as the case may be, under conditions effective to allow the formation of immunocomplexes.

The immunobinding methods include methods for detecting or quantifying the amount of a reactive component in a sample, which methods require the detection or quantitation of any immune complexes formed during the binding process. Here, one would obtain a sample suspected of containing a prostate specific protein, peptide or a corresponding antibody, and contact the sample with an antibody or encoded protein or peptide, as the case may be, and then detect or quantify the amount of immune complexes formed under the specific conditions.

In terms of biomarker detection, the biological sample analyzed may be any sample that is suspected of containing PDIA3. Contacting the chosen biological sample with the protein (e.g., PDIA3 or antigen thereof to bind with an anti-PDIA3 antibody in the blood), peptide (e.g., PDIA3 fragment that binds with an anti-PDIA3 antibody in the blood), or antibody (e.g., as a detection reagent that binds PDIA3 in a biological sample) under conditions effective and for a period of time sufficient to allow the formation of immune complexes (primary immune complexes). Generally, complex formation is a matter of simply adding the composition to the biological sample and incubating the mixture for a period of time long enough for the antibodies to form immune complexes with, i.e., to bind to, any antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The encoded protein (e.g., PDIA3), peptide (e.g., PDIA3 peptide) or corresponding antibody (anti-PDIA3 antibody as detection reagent) employed in the detection may itself be linked to a detectable label, wherein one would then simply detect this label, thereby allowing the amount of the primary immune complexes in the composition to be determined.

Alternatively, the first added component that becomes bound within the primary immune complexes may be detected by means of a second binding ligand that has binding affinity for the encoded protein, peptide or corresponding antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the encoded protein, peptide or corresponding antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

The immunodetection methods of the present invention have evident utility in the diagnosis of conditions such as prostate cancer. Here, a biological or clinical sample suspected of containing either the encoded protein or peptide or corresponding antibody is used. However, these embodiments also have applications to non-clinical samples, such as in the tittering of antigen or antibody samples, in the selection of hybridomas, and the like.

The present invention, in particular, contemplates the use of ELISAs as a type of immunodetection assay. It is contemplated that the biomarker proteins or peptides of the invention will find utility as immunogens in ELISA assays in diagnosis and prognostic monitoring of prostate cancer. Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and Western blotting, dot blotting, FACS analyses, and the like also may be used.

In one exemplary ELISA, antibodies binding to the biomarkers of the invention are immobilized onto a selected surface exhibiting protein affinity, such as a well in a polystyrene microtiter plate. Then, a test composition suspected of containing the prostate cancer marker antigen, such as a clinical sample, is added to the wells. After binding and washing to remove non-specifically bound immunecomplexes, the bound antigen may be detected. Detection is generally achieved by the addition of a second antibody specific for the target protein, that is linked to a detectable label. This type of ELISA is a simple “sandwich ELISA.” Detection also may be achieved by the addition of a second antibody, followed by the addition of a third antibody that has binding affinity for the second antibody, with the third antibody being linked to a detectable label.

In another exemplary ELISA, the samples suspected of containing the prostate cancer marker antigen are immobilized onto the well surface and then contacted with the anti-biomarker antibodies of the invention. After binding and washing to remove non-specifically bound immunecomplexes, the bound antigen is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Irrespective of the format employed, ELISAs have certain features in common, such as coating, incubating or binding, washing to remove non-specifically bound species, and detecting the bound immunecomplexes. These are described as follows.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control human prostate, cancer and/or clinical or biological sample to be tested under conditions effective to allow immunecomplex (antigen/antibody) formation. Detection of the immunecomplex then requires a labeled secondary binding ligand or antibody, or a secondary binding ligand or antibody in conjunction with a labeled tertiary antibody or third binding ligand.

The phrase “under conditions effective to allow immunecomplex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 h, at temperatures preferably on the order of 25 to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immunecomplex with a urease, glucose oxidase, alkaline phosphatase or hydrogen peroxidase-conjugated antibody for a period of time and under conditions that favor the development of further immunecomplex formation (e.g., incubation for 2 h at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple. Quantitation is then achieved by measuring the degree of color generation, e.g., using a visible spectra spectrophotometer.

PDIA3 can also be measured, quantitated, detected, and otherwise analyzed using protein mass spectrometry methods and instrumentation. Protein mass spectrometry refers to the application of mass spectrometry to the study of proteins. Although not intending to be limiting, two approaches are typically used for characterizing proteins using mass spectrometry. In the first, intact proteins are ionized and then introduced to a mass analyzer. This approach is referred to as “top-down” strategy of protein analysis. The two primary methods for ionization of whole proteins are electrospray ionization (ESI) and matrix-assisted laser desorption/ionization (MALDI). In the second approach, proteins are enzymatically digested into smaller peptides using a protease such as trypsin. Subsequently these peptides are introduced into the mass spectrometer and identified by peptide mass fingerprinting or tandem mass spectrometry. Hence, this latter approach (also called “bottom-up” proteomics) uses identification at the peptide level to infer the existence of proteins.

Whole protein mass analysis of the biomarkers of the invention can be conducted using time-of-flight (TOF) MS, or Fourier transform ion cyclotron resonance (FT-ICR). These two types of instruments are useful because of their wide mass range, and in the case of FT-ICR, its high mass accuracy. The most widely used instruments for peptide mass analysis are the MALDI time-of-flight instruments as they permit the acquisition of peptide mass fingerprints (PMFs) at high pace (1 PMF can be analyzed in approx. 10 sec). Multiple stage quadrupole-time-of-flight and the quadrupole ion trap also find use in this application.

The PDIA3 can also be measured in complex mixtures of proteins and molecules that co-exist in a biological medium or sample, however, fractionation of the sample may be required and is contemplated herein. It will be appreciated that ionization of complex mixtures of proteins can result in situation where the more abundant proteins have a tendency to “drown” or suppress signals from less abundant proteins in the same sample. In addition, the mass spectrum from a complex mixture can be difficult to interpret because of the overwhelming number of mixture components. Fractionation can be used to first separate any complex mixture of proteins prior to mass spectrometry analysis. Two methods are widely used to fractionate proteins, or their peptide products from an enzymatic digestion. The first method fractionates whole proteins and is called two-dimensional gel electrophoresis. The second method, high performance liquid chromatography (LC or HPLC) is used to fractionate peptides after enzymatic digestion. In some situations, it may be desirable to combine both of these techniques. Any other suitable methods known in the art for fractionating protein mixtures are also contemplated herein.

Gel spots identified on a 2D Gel are usually attributable to one protein. If the identity of the protein is desired, usually the method of in-gel digestion is applied, where the protein spot of interest is excised, and digested proteolytically. The peptide masses resulting from the digestion can be determined by mass spectrometry using peptide mass fingerprinting. If this information does not allow unequivocal identification of the protein, its peptides can be subject to tandem mass spectrometry for de novo sequencing.

Characterization of protein mixtures using HPLC/MS may also be referred to in the art as “shotgun proteomics” and MuDPIT (Multi-Dimensional Protein Identification Technology). A peptide mixture that results from digestion of a protein mixture is fractionated by one or two steps of liquid chromatography (LC). The eluent from the chromatography stage can be either directly introduced to the mass spectrometer through electrospray ionization, or laid down on a series of small spots for later mass analysis using MALDI.

PDIA3 can be identified using MS using a variety of techniques, all of which are contemplated herein. Peptide mass fingerprinting uses the masses of proteolytic peptides as input to a search of a database of predicted masses that would arise from digestion of a list of known proteins. If a protein sequence in the reference list gives rise to a significant number of predicted masses that match the experimental values, there is some evidence that this protein was present in the original sample. It will be further appreciated that the development of methods and instrumentation for automated, data-dependent electrospray ionization (ESI) tandem mass spectrometry (MS/MS) in conjunction with microcapillary liquid chromatography (LC) and database searching has significantly increased the sensitivity and speed of the identification of gel-separated proteins. Microcapillary LC-MS/MS has been used successfully for the large-scale identification of individual proteins directly from mixtures without gel electrophoretic separation (Link et al., 1999; Opitek et al., 1997).

Several recent methods allow for the quantitation of proteins by mass spectrometry. For example, stable (e.g., non-radioactive) heavier isotopes of carbon (¹³C) or nitrogen (¹⁵N) can be incorporated into one sample while the other one can be labeled with corresponding light isotopes (e.g. ¹²C and ¹⁴N). The two samples are mixed before the analysis. Peptides derived from the different samples can be distinguished due to their mass difference. The ratio of their peak intensities corresponds to the relative abundance ratio of the peptides (and proteins). The most popular methods for isotope labeling are SILAC (stable isotope labeling by amino acids in cell culture), trypsin-catalyzed ¹⁸O labeling, ICAT (isotope coded affinity tagging), iTRAQ (isobaric tags for relative and absolute quantitation). “Semi-quantitative” mass spectrometry can be performed without labeling of samples. Typically, this is done with MALDI analysis (in linear mode). The peak intensity, or the peak area, from individual molecules (typically proteins) is here correlated to the amount of protein in the sample. However, the individual signal depends on the primary structure of the protein, on the complexity of the sample, and on the settings of the instrument. Other types of “label-free” quantitative mass spectrometry, uses the spectral counts (or peptide counts) of digested proteins as a means for determining relative protein amounts.

PDIA3 can be identified and quantified from a complex biological sample using mass spectroscopy in accordance with the following exemplary method, which is not intended to limit the invention or the use of other mass spectrometry-based methods.

In the first step of this embodiment, (A) a biological sample which comprises a complex mixture of protein (including at least one biomarker of interest) is fragmented and labeled with a stable isotope X. (B) Next, a known amount of an internal standard is added to the biological sample, wherein the internal standard is prepared by fragmenting a standard protein that is identical to the at least one target biomarker of interest, and labeled with a stable isotope Y. (C) This sample obtained is then introduced in an LC-MS/MS device, and multiple reaction monitoring (MRM) analysis is performed using MRM transitions selected for the internal standard to obtain an MRM chromatogram. (D) The MRM chromatogram is then viewed to identify a target peptide biomarker derived from the biological sample that shows the same retention time as a peptide derived from the internal standard (an internal standard peptide), and quantifying the target protein biomarker in the test sample by comparing the peak area of the internal standard peptide with the peak area of the target peptide biomarker.

Any suitable biological sample may be used as a starting point for LC-MS/MS/MRM analysis, including biological samples derived blood, urine, saliva, hair, cells, cell tissues, biopsy materials, and treated products thereof; and protein-containing samples prepared by gene recombination techniques.

Each of the above steps (A) to (D) is described further below.

Step (A) (Fragmentation and Labeling). In step (A), the target protein biomarker is fragmented to a collection of peptides, which is subsequently labeled with a stable isotope X. To fragment the target protein, for example, methods of digesting the target protein with a proteolytic enzyme (protease) such as trypsin, and chemical cleavage methods, such as a method using cyanogen bromide, can be used. Digestion by protease is preferable. It is known that a given mole quantity of protein produces the same mole quantity for each tryptic peptide cleavage product if the proteolytic digest is allowed to proceed to completion. Thus, determining the mole quantity of tryptic peptide to a given protein allows determination of the mole quantity of the original protein in the sample. Absolute quantification of the target protein can be accomplished by determining the absolute amount of the target protein-derived peptides contained in the protease digestion (collection of peptides). Accordingly, in order to allow the proteolytic digest to proceed to completion, reduction and alkylation treatments are preferably performed before protease digestion with trypsin to reduce and alkylate the disulfide bonds contained in the target protein.

Subsequently, the obtained digest (collection of peptides, comprising peptides of the target biomarker in the biological sample) is subjected to labeling with a stable isotope X. Examples of stable isotopes X include ¹H and ²H for hydrogen atoms, ¹²C and ¹³C for carbon atoms, and ¹⁴N and ¹⁵N for nitrogen atoms. Any isotope can be suitably selected therefrom. Labeling by a stable isotope X can be performed by reacting the digest (collection of peptides) with a reagent containing the stable isotope. Preferable examples of such reagents that are commercially available include mTRAQ (registered trademark) (produced by Applied Biosystems), which is an amine-specific stable isotope reagent kit. mTRAQ is composed of 2 or 3 types of reagents (mTRAQ-light and mTRAQ-heavy; or mTRAQ-D0, mTRAQ-D4, and mTRAQ-D8) that have a constant mass difference therebetween as a result of isotope-labeling, and that are bound to the N-terminus of a peptide or the primary amine of a lysine residue.

Step (B) (Addition of the Internal Standard). In step (B), a known amount of an internal standard is added to the sample obtained in step (A). The internal standard used herein is a digest (collection of peptides) obtained by fragmenting a protein (standard protein) consisting of the same amino acid sequence as the target protein (target biomarker) to be measured, and labeling the obtained digest (collection of peptides) with a stable isotope Y. The fragmentation treatment can be performed in the same manner as above for the target protein. Labeling with a stable isotope Y can also be performed in the same manner as above for the target protein. However, the stable isotope Y used herein must be an isotope that has a mass different from that of the stable isotope X used for labeling the target protein digest. For example, in the case of using the aforementioned mTRAQ (registered trademark) (produced by Applied Biosystems), when mTRAQ-light is used to label a target protein digest, mTRAQ-heavy should be used to label a standard protein digest.

Step (C) (LC-MS/MS and MRM Analysis). In step (C), the sample obtained in step (B) is first placed in an LC-MS/MS device, and then multiple reaction monitoring (MRM) analysis is performed using MRM transitions selected for the internal standard. By LC (liquid chromatography) using the LC-MS/MS device, the sample (collection of peptides labeled with a stable isotope) obtained in step (B) is separated first by one-dimensional or multi-dimensional high-performance liquid chromatography. Specific examples of such liquid chromatography include cation exchange chromatography, in which separation is conducted by utilizing electric charge difference between peptides; and reversed-phase chromatography, in which separation is conducted by utilizing hydrophobicity difference between peptides. Both of these methods may be used in combination.

Subsequently, each of the separated peptides is subjected to tandem mass spectrometry by using a tandem mass spectrometer (MS/MS spectrometer) comprising two mass spectrometers connected in series. The use of such a mass spectrometer enables the detection of several fmol levels of a target protein. Furthermore, MS/MS analysis enables the analysis of internal sequence information on peptides, thus enabling identification without false positives. Other types of MS analyzers may also be used, including magnetic sector mass spectrometers (Sector MS), quadrupole mass spectrometers (QMS), time-of-flight mass spectrometers (TOFMS), and Fourier transform ion cyclotron resonance mass spectrometers (FT-ICRMS), and combinations of these analyzers.

Subsequently, the obtained data are put through a search engine to perform a spectral assignment and to list the peptides experimentally detected for each protein. The detected peptides are preferably grouped for each protein, and preferably at least three fragments having an m/z value larger than that of the precursor ion and at least three fragments with an m/z value of, preferably, 500 or more are selected from each MS/MS spectrum in descending order of signal strength on the spectrum. From these, two or more fragments are selected in descending order of strength, and the average of the strength is defined as the expected sensitivity of the MRR transitions. When a plurality of peptides is detected from one protein, at least two peptides with the highest sensitivity are selected as standard peptides using the expected sensitivity as an index.

Step (D) (Quantification of the Target Protein in the Test Sample). Step (D) comprises identifying, in the MRM chromatogram detected in step (C), a peptide derived from the target protein (a target biomarker of interest) that shows the same retention time as a peptide derived from the internal standard (an internal standard peptide), and quantifying the target protein in the test sample by comparing the peak area of the internal standard peptide with the peak area of the target peptide. The target protein can be quantified by utilizing a calibration curve of the standard protein prepared beforehand.

The calibration curve can be prepared by the following method. First, a recombinant protein consisting of an amino acid sequence that is identical to that of the target biomarker protein is digested with a protease such as trypsin, as described above. Subsequently, precursor-fragment transition selection standards (PFTS) of a known concentration are individually labeled with two different types of stable isotopes (i.e., one is labeled with a stable isomer used to label an internal standard peptide (labeled with IS), whereas the other is labeled with a stable isomer used to label a target peptide (labeled with T). A plurality of samples are produced by blending a certain amount of the IS-labeled PTFS with various concentrations of the T-labeled PTFS. These samples are placed in the aforementioned LC-MS/MS device to perform MRM analysis. The area ratio of the T-labeled PTFS to the IS-labeled PTFS (T-labeled PTFS/IS-labeled PTFS) on the obtained MRM chromatogram is plotted against the amount of the T-labeled PTFS to prepare a calibration curve. The absolute amount of the target protein contained in the test sample can be calculated by reference to the calibration curve.

3. Antibodies and Labels

In some embodiments, the invention provides methods and compositions that include labels for the highly sensitive detection and quantitation of PDIA3. One skilled in the art will recognize that many strategies can be used for labeling target molecules to enable their detection or discrimination in a mixture of particles (e.g., labeled anti-PDIA3 antibody or labeled secondary antibody, or labeled oligonucleotide probe that specifically hybridizes to PDIA3 mRNA). The labels may be attached by any known means, including methods that utilize non-specific or specific interactions of label and target. Labels may provide a detectable signal or affect the mobility of the particle in an electric field. In addition, labeling can be accomplished directly or through binding partners.

In some embodiments, the label comprises a binding partner that binds to the biomarker of interest, where the binding partner is attached to a fluorescent moiety. The compositions and methods of the invention may utilize highly fluorescent moieties, e.g., a moiety capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. Moieties suitable for the compositions and methods of the invention are described in more detail below.

In some embodiments, the invention provides a label for detecting a biological molecule comprising a binding partner for the biological molecule that is attached to a fluorescent moiety, wherein the fluorescent moiety is capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. In some embodiments, the moiety comprises a plurality of fluorescent entities, e.g., about 2 to 4, 2 to 5, 2 to 6, 2 to 7, 2 to 8, 2 to 9, 2 to 10, or about 3 to 5, 3 to 6, 3 to 7, 3 to 8, 3 to 9, or 3 to 10 fluorescent entities. In some embodiments, the moiety comprises about 2 to 4 fluorescent entities. In some embodiments, the biological molecule is a protein or a small molecule. In some embodiments, the biological molecule is a protein. The fluorescent entities can be fluorescent dye molecules. In some embodiments, the fluorescent dye molecules comprise at least one substituted indolium ring system in which the substituent on the 3-carbon of the indolium ring contains a chemically reactive group or a conjugated substance. In some embodiments, the dye molecules are Alexa Fluor molecules selected from the group consisting of Alexa Fluor 488, Alexa Fluor 532, Alexa Fluor 647, Alexa Fluor 680 or Alexa Fluor 700. In some embodiments, the dye molecules are Alexa Fluor molecules selected from the group consisting of Alexa Fluor 488, Alexa Fluor 532, Alexa Fluor 680 or Alexa Fluor 700. In some embodiments, the dye molecules are Alexa Fluor 647 dye molecules. In some embodiments, the dye molecules comprise a first type and a second type of dye molecules, e.g., two different Alexa Fluor molecules, e.g., where the first type and second type of dye molecules have different emission spectra. The ratio of the number of first type to second type of dye molecule can be, e.g., 4 to 1, 3 to 1, 2 to 1, 1 to 1, 1 to 2, 1 to 3 or 1 to 4. The binding partner can be, e.g., an antibody.

In some embodiments, the invention provides a label for the detection of a biological marker of the invention, wherein the label comprises a binding partner for the marker and a fluorescent moiety, wherein the fluorescent moiety is capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. In some embodiments, the fluorescent moiety comprises a fluorescent molecule. In some embodiments, the fluorescent moiety comprises a plurality of fluorescent molecules, e.g., about 2 to 10, 2 to 8, 2 to 6, 2 to 4, 3 to 10, 3 to 8, or 3 to 6 fluorescent molecules. In some embodiments, the label comprises about 2 to 4 fluorescent molecules. In some embodiments, the fluorescent dye molecules comprise at least one substituted indolium ring system in which the substituent on the 3-carbon of the indolium ring contains a chemically reactive group or a conjugated substance. In some embodiments, the fluorescent molecules are selected from the group consisting of Alexa Fluor 488, Alexa Fluor 532, Alexa Fluor 647, Alexa Fluor 680 or Alexa Fluor 700. In some embodiments, the fluorescent molecules are selected from the group consisting of Alexa Fluor 488, Alexa Fluor 532, Alexa Fluor 680 or Alexa Fluor 700. In some embodiments, the fluorescent molecules are Alexa Fluor 647 molecules. In some embodiments, the binding partner comprises an antibody. In some embodiments, the antibody is a monoclonal antibody. In other embodiments, the antibody is a polyclonal antibody.

In various embodiments, the binding partner for detecting PDIA3 is an antibody or antigen-binding fragment thereof. The term “antibody,” as used herein, is a broad term and is used in its ordinary sense, including, without limitation, to refer to naturally occurring antibodies as well as non-naturally occurring antibodies, including, for example, single chain antibodies, chimeric, bifunctional and humanized antibodies, as well as antigen-binding fragments thereof. An “antigen-binding fragment” of an antibody refers to the part of the antibody that participates in antigen binding. The antigen binding site is formed by amino acid residues of the N-terminal variable (“V”) regions of the heavy (“H”) and light (“L”) chains. It will be appreciated that the choice of epitope or region of the molecule to which the antibody is raised will determine its specificity, e.g., for various forms of the molecule, if present, or for total (e.g., all, or substantially all of the molecule).

Methods for producing antibodies are well-established. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)). Monoclonal and polyclonal antibodies to molecules, e.g., proteins, and markers also commercially available (R and D Systems, Minneapolis, Minn.; HyTest, HyTest Ltd., Turku Finland; Abcam Inc., Cambridge, Mass., USA, Life Diagnostics, Inc., West Chester, Pa., USA; Fitzgerald Industries International, Inc., Concord, Mass. 01742-3049 USA; BiosPacific, Emeryville, Calif.).

In some embodiments, the antibody is a polyclonal antibody. In other embodiments, the antibody is a monoclonal antibody.

In still other embodiments, particularly where oligonucleotides are used as binding partners to detect and hybridize to mRNA biomarkers or other nucleic acid based biomarkers, the binding partners (e.g., oligonucleotides) can comprise a label, e.g., a fluorescent moiety or dye. In addition, any binding partner of the invention, e.g., an antibody, can also be labeled with a fluorescent moiety. The fluorescence of the moiety will be sufficient to allow detection in a single molecule detector, such as the single molecule detectors described herein. A “fluorescent moiety,” as that term is used herein, includes one or more fluorescent entities whose total fluorescence is such that the moiety may be detected in the single molecule detectors described herein. Thus, a fluorescent moiety may comprise a single entity (e.g., a Quantum Dot or fluorescent molecule) or a plurality of entities (e.g., a plurality of fluorescent molecules). It will be appreciated that when “moiety,” as that term is used herein, refers to a group of fluorescent entities, e.g., a plurality of fluorescent dye molecules, each individual entity may be attached to the binding partner separately or the entities may be attached together, as long as the entities as a group provide sufficient fluorescence to be detected.

Kits/Panels

The invention also provides compositions and kits for measuring the level of PDIA3 in a biological sample from a subject, e.g., a subject having cancer and who is in need of being treated for the cancer with Coenzyme Q10. These kits include one or more of the following: a detectable antibody that specifically binds to PDIA3, reagents for obtaining and/or preparing subject tissue samples for staining, and instructions for use.

The invention also encompasses kits for detecting the presence of a PDIA3 protein or nucleic acid in a biological sample. Such kits can be used to predict if a subject suffering from a cancer will be responsive to treatment with Coenzyme Q10. Such kits can also be used to select a subject for treatment with Coenzyme Q10. For example, the kit can comprise a labeled compound or agent capable of detecting a PDIA3 protein or nucleic acid in a biological sample and means for determining the amount of the protein or mRNA in the sample (e.g., an antibody which binds the protein or a fragment thereof, or an oligonucleotide probe which binds to DNA or mRNA encoding the protein). Kits can also include instructions for use of the kit for practicing any of the methods provided herein or interpreting the results obtained using the kit based on the teachings provided herein. The kits can also include reagents for detection of a control protein in the sample, e.g., actin for tissue samples, albumin in blood or blood derived samples, for normalization of the amount of the marker present in the sample. The kit can also include the purified marker for detection for use as a control or for quantitation of the assay performed with the kit.

For antibody-based kits, the kit can comprise, for example: (1) a first antibody (e.g., attached to a solid support) which binds to PDIA3 protein; and, optionally, (2) a second, different antibody which binds to either PDIA3 or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a PDIA3 protein or (2) a pair of primers useful for amplifying the marker nucleic acid molecule.

For chromatography methods, the kit can include markers, including labeled markers, to permit detection and identification of PDIA3 by chromatography. In certain embodiments, kits for chromatography methods include compounds for derivatization of PDIA3. In certain embodiments, kits for chromatography methods include columns for resolving the markers of the method.

Reagents specific for detection of PDIA3 allow for detection and quantitation of the marker in a complex mixture, e.g., serum, tissue sample. In certain embodiments, the reagents are species specific. In certain embodiments, the reagents are not species specific. In certain embodiments, the reagents are isoform specific. In certain embodiments, the reagents are not isoform specific. In certain embodiments, the reagents detect total PDIA3.

In certain embodiments, the kits for the detection of PDIA3 in a biological sample from a subject, e.g, a subject having cancer and in need of treatment with CoQ10, comprise at least one reagent specific for the detection of the level of expression of PDIA3. In certain embodiments, the kits further comprise instructions for comparing the level of PDIA3 in the biological sample from the subject to a threshold value of PDIA3. In certain embodiments, the kits further comprise instructions for the identification of a subject who is predicted to be responsive to CoQ10 based on the level of expression of PDIA3, e.g., a level above a threshold value. In certain embodiments, the kits further comprise instructions for the selection of a subject for treatment with CoQ10 based on the level of expression of PDIA3, e.g., a level above a threshold value.

In certain embodiments, the kits can also comprise, e.g., a buffering agents, a preservative, a protein stabilizing agent, reaction buffers. The kit can further comprise components necessary for detecting the detectable label (e.g., an enzyme or a substrate). The kit can also contain a control sample or a series of control samples which can be assayed and compared to the test sample. The controls can be control serum samples or control samples of purified proteins or nucleic acids, as appropriate, with known levels of target markers. Each component of the kit can be enclosed within an individual container and all of the various containers can be within a single package, along with instructions for interpreting the results of the assays performed using the kit. The kits of the invention may optionally comprise additional components useful for performing the methods of the invention.

This invention is further illustrated by the following examples which should not be construed as limiting. The contents of all references and published patents and patent applications cited throughout the application are hereby incorporated by reference.

### EXAMPLE 1

### Identification of Candidate Biomarkers in an ongoing Phase I Clinical Trial of Coenzyme Q10 for Treatment of Advanced Solid Tumors

Patients enrolled in an ongoing Phase I clinical trial of Coenzyme Q10 for treatment of advanced solid tumors were evaluated to identify candidate biomarkers to guide the use of Coenzyme Q10 for the treatment of cancer. This example includes preliminary analysis conducted while the trial was ongoing. Example 2 includes a more in depth analysis conducted at a later period in the same clinical trial when more patients were enrolled and more data was available.

Trial Design

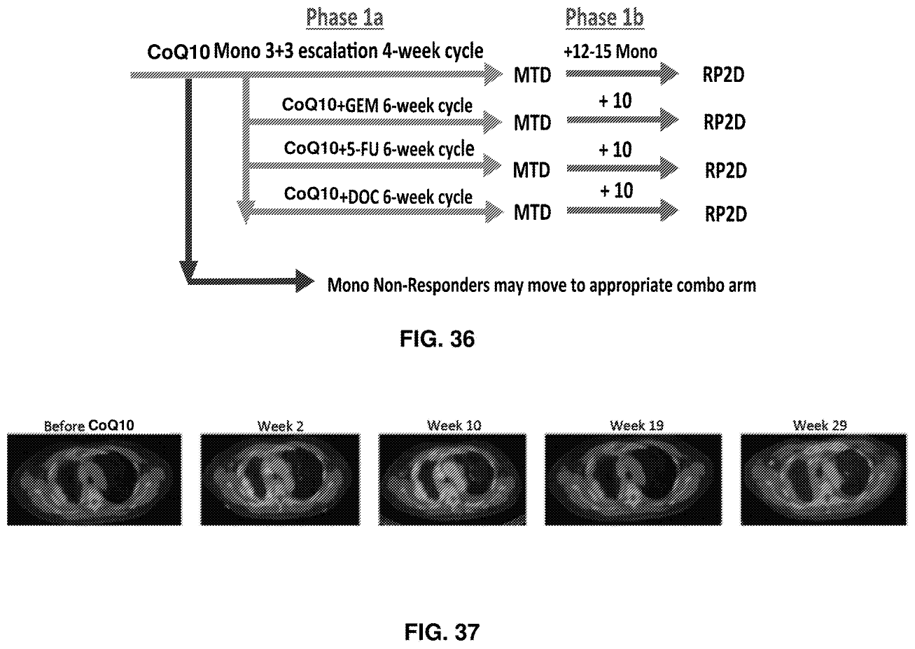

The clinical trial is a multicenter, open-label, non-randomized, dose-escalation study to examine the dose limiting toxicities (DLT) of Coenzyme Q10 administered as a 144-hour continuous intravenous (IV) infusion as monotherapy (treatment Arm 1) and in combination with chemotherapy (treatment Arm 2) in patients with solid tumors. A broad range of solid tumors has been evaluated, including prostate, colon, breast, lung and pancreatic tumors, as shown in Tables 1 and 2 below. Coenzyme Q10 was administered in three consecutive 48 hour doses or two consecutive 72 hour doses, depending on the dose level. Three standard weekly chemotherapy regimens of gemcitabine, 5-fluorouracil, or docetaxel were evaluated in combination with Coenzyme Q10. Eligible patients are 18 years of age or older, afflicted with solid tumors, and relapsed/refractory to standard therapy. 85 patients have been enrolled in the trial. The monotherapy arm received Coenzyme Q10 for 6 days in continuous infusion in 28 day cycles, and the combination arms (gemcitabine, 5-fluorouracil, or docetaxel) were primed for 3 weeks with Coenzyme Q10 before initiation of standard chemotherapy, followed by weekly dosing in a 6 week cycle. A summary of the treatment groups is shown in FIG. 36.

The study is a standard 3+3 dose escalation design with the dose escalated in successive cohorts of 3 to 6 patients each. Toxicity at each dose level is graded according to National Cancer Institute Common Terminology Criteria for Adverse Events (CTCAE v4.02). Safety oversight is provided by the Cohort Review Committee (CRC). If none of the 3 patients in a cohort experiences a DLT during Cycle 1, then 3 new patients may be entered at the next higher dose level following CRC review of safety and PK data from lower cohorts. The clinical trial is described in greater detail in WO2015/035094, which is incorporated by reference herein in its entirety.

Patient Evaluation

Tumor response was evaluated at week 2 and then after every 2 cycles. Sixteen of 66 patients (24%) maintained a minimum of Stable Disease for ≥4 cycles. Tumor response data was used to stratify the patients into “overall clinical benefit” or “no clinical benefit” groups.

Blood samples were collected from the patients at several time points throughout the trial. Blood samples were centrifuged to obtain plasma/serum and the buffy coat (containing white blood cells and platelets) for further analysis. Urine samples were collected during Cycle 1 of monotherapy and combination therapy. PET scans with fluorodeoxyglucose (FDG) uptake and cancer biopsies were performed 2 weeks prior to starting Coenzyme Q10 treatment and 2 weeks after initiation of Coenzyme Q10 treatment. FDG-PET scans were used to evaluate tumor response to Coenzyme Q10, and may also be used to determine the metabolic status of the tumor. For example, FIG. 37 shows FDG-PET scans before and 2, 10, 19 and 29 weeks after Coenzyme Q10 monotherapy in a patient with metastatic appendiceal cancer with surgery and heavily pretreated with multiple FOLFIRI and FOLFOX regimens in combination with irinotecan and Avastin, respectively. Coenzyme Q10 monotherapy was initiated at 66 mg/kg dose and moved to 88 mg/kg dose at 22 weeks.

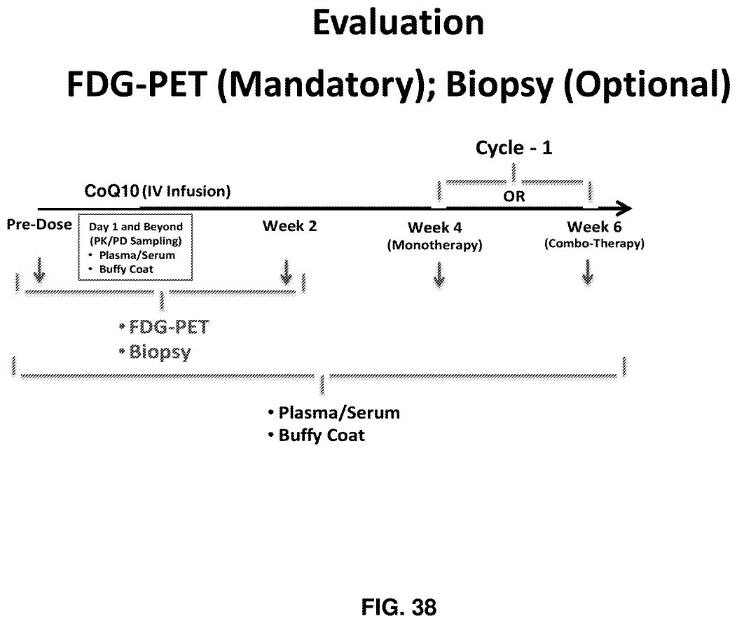

An overview of the schedule for sampling and FDG PET-scans is provided in FIG. 38.

A broad range of clinical data was recorded for each patient, including the dose limiting toxicities (DLTs), pharmacokinetics (pK) and adverse events described below. The clinical data also included demographic data such as age, gender and ethnicity; tumor status as described above; and medical history including the type and location of the tumor and previous medical treatments.

Dose Limiting Toxicities

DLTs were reported at 171 mg/kg in the Coenzyme Q10 monotherapy arm and at 137 mg/kg in the gemcitabine arm (maximum administered dose) and were coagulopathy-related. See Tables 1, 2 and 3 below. 3 DLTs were reported during the time period covered by Example 1. 1 DLT (grade 3 partial thromboplastin time (PTT) abnormality) was reported in the Mono Dose Level 5 (171 mg/kg). The event resolved in 2 days after administration of Vitamin K and fresh frozen plasma (FFP). Three additional patients were enrolled at this dose level with no additional DLTs reported. 2 DLTs (grade 3 aspartate transaminase (AST) elevation and grade 4 thrombocytopenia) were reported in the combination dose level 137 mg/kg with gemcitabine. According to trial design, patients were being enrolled into the next lowest dose level (110 mg/kg).

The most common related adverse events were grade 1-2 prothrombin time (PT) /partial thromboplastin time(PTT)/International Normalized Ratio(INR) prolongation that were mitigated after Vitamin K administration. Four grade 3 events were reported. During the time period covered by Example 1, 1503 adverse events were reported. 75 events were reported as serious. Of the serious adverse events, 27 were not related, 38 were unlikely related, 8 were possibly related, one was probably related and, one was definitely related (activated partial thromboplastin time (APTT) prolonged).

Pharmacokinetics

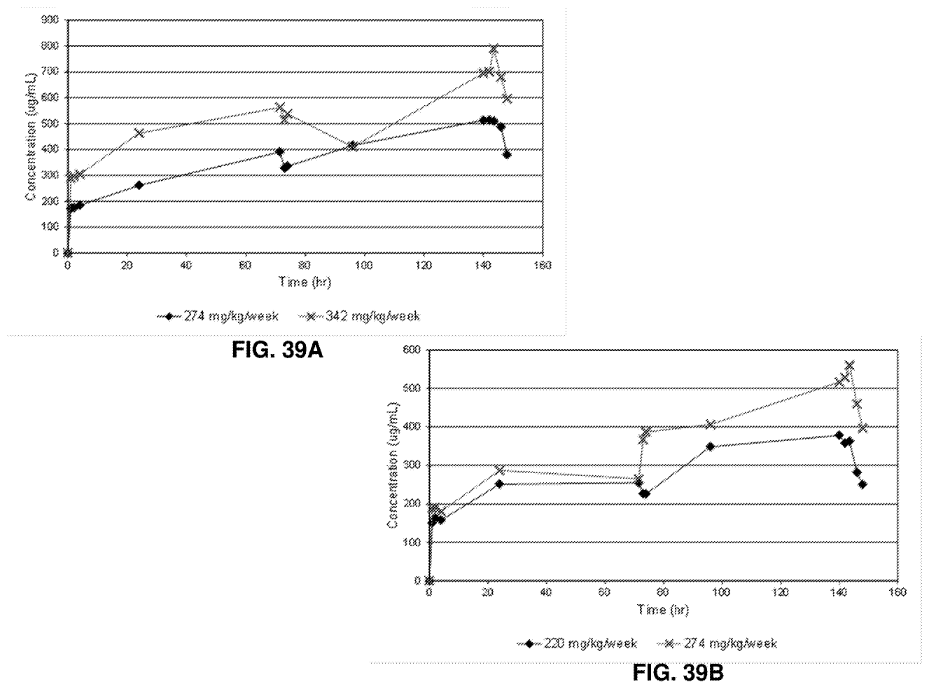

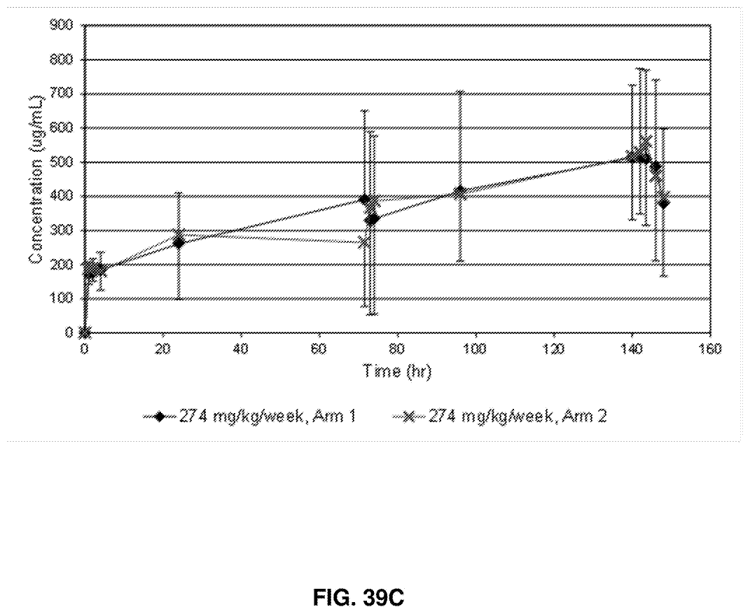

Pharmacokinetics of Coenzyme Q10 was measured in the patients at time zero and at several time points during and after the 144-hour continuous intravenous (IV) infusion with Coenzyme Q10. For Arm 1 (monotherapy), the mean concentrations of Coenzyme Q10 were higher for the 342 mg/kg/week dose than for the 274 mg/kg/week dose, with the exception of the 96-hour sampling time when the mean concentrations of Coenzyme Q10 were similar For Arm 2 (chemotherapy combination therapy), the plasma profiles were slightly higher for the 274 mg/kg/week dose than for the 220 mg/kg/week dose during the first 72 hours of the infusion, and distinctly higher for the 274 mg/kg/week dose during the second 72 hours of the infusion. See FIGS. 39A-39C and Table 5. There were no clear differences between the pharmacokinetic profiles for Arm 1 and Arm 2 at any of the dose levels, indicating no apparent effect of concomitant chemotherapy on the pharmacokinetics of Coenzyme Q10.

Table 4. Dose limiting toxicities for Coenzyme Q10 monotherapy. The number of patients enrolled at each dose level (DL) is shown in parentheses. DL4 and DL5 were administered in two consecutive 72 hour IV infusions. All other dose levels were administered by three consecutive 48 hour IV infusions.

TABLE 4
Dose limiting toxicities for Coenzyme
Q10 monotherapy.

| Dose Level | | Patients | |
|---|---|---|---|
| Monotherapy | | Evaluable | Dose Limiting |
| (N = 30) | Tumor Type | for DLT | Toxicity |
| DL1-66 | Gastric, Colon (3), | 6 | Grade 3 |
| mg/kg (9) | Prostate, SCC, | | Elevated Liver |
| | Right Tonsil, Gall | | Function Test* |
| | Bladder, Appendicle, | | |
| | Soft Tissue | | |
| | Sarcoma | | |
| DL2-88 | Carcinoid, Rectal, | 3 | None |
| mg/kg (4) | Ovarian, Breast | | |
| DL3-110 | Renal, Esophageal | 3 | None |
| mg/kg (5) | SCC, Pancreatic, | | |
| | Non-small cell lung, | | |
| | Colon | | |
| DL4-137 | Tongue, Bladder, | 3 | None |
| mg/kg (4) | Angiosarcoma, | | |
| | Hepatocellular | | |
| DL5-171 | Colorectal, Chondrosarcoma, | 6 | 1 DLT: Grade |
| mg/kg (8) | Unk Primary, Appendiceal, | | 3 PTT |
| | Hepatocellular, | | elevation |
| | Breast, Adenoid Cystic Sarcoma, | | |
| | Anaplastic Astrocytoma | | |

*The toxicity was readjudicated to unlikely related to protocol therapy and likely related to disease progression.

The table below lists dose limiting toxicities for Coenzyme Q10 combination therapy with gemcitabine, 5-fluorouracil (5FU) or docetaxel. The number of patients enrolled at each dose level (DL) is shown in parentheses. DL4 and DL5 were administered with two consecutive 72 hour infusions. All other dose levels were administered with three consecutive 48 hour infusions. All 5FU dose levels include leucovorin at 100 mg/m².

TABLE 5
Dose limiting toxicities for Coenzyme Q10 combination therapy with
gemcitabine, 5-fluorouracil (5FU) or docetaxel.

| Dose Level Arm 2 | | Evaluable | Dose Limiting |
|---|---|---|---|
| (N = 55) | Tumor Type | for DLT | Toxicity |
| DL1-50 mg/kg with: | | | |
| Gemcitibine 600 mg/m² | Pancreatic, Neuroendocrine, | 3 | None |
| (3) | Breast | | |
| 5FU 350 mg/m² (3) | Colon (2), SCC of Head and | 3 | None |
| | Neck | | |
| Docetaxel 20 mg/m² (3) | Lung, Uterine Leiomyosarcoma, | 3 | None |
| | Ovarian | | |
| DL2-66 mg/kg with: | | | |
| Gemcitabine 600 | Ovarian, Peritoneal | 3 | None |
| mg/m² (6) | Mesothelioma, Bladder, Breast, | | |
| | Espophageal, Lung | | |
| 5FU 350 mg/m² (3) | Colon (3) | 3 | None |
| Docetaxel 20 mg/m² (3) | Lung (2), Breast | 3 | None |
| DL3-88 mg/kg with: | | | |
| Gemcitabine 800 | Squamous Cell Head and Neck, | 3 | None |
| mg/m² (3) | Pancreatic, Lung | | |
| | Esophageal, | | |
| | Cholangiocardinoma, | | |
| 5FU 450 mg/m² (4) | Hemangiopericytoma of the | 3 | None |
| | Pelvis, Colon | | |
| Docetaxel 25 mg/m² (7) | JE Junction, Breast (2), | 3 | None |
| | Cholangiocarcinoma, Maxillary | | |
| | Sarcoma, Ampullary Carcinoma, | | |
| | Tongue | | |
| DL4-110 mg/kg with: | | | |
| Gemcitabine 1,000 | Lung (2), Leiomyosarcoma, | 3 | None to Date-need |
| mg/m² (6) | Appendicile, Colon, | | 3 more evaluable |
| | Osteosarcoma | | patients to |
| | | | determine MTD |
| 5FU 500 mg/m² (4) | Spindle Cell Sarcoma, Urachal | 3 | None |
| | Carcinoma, Colon, Rectal | | |
| | Esophageal, Nasopharangeal | | |
| Docetaxel 30 mg/m² (4) | Sarcoma, Leiomyosarcoma, | 3 | None |
| | Endometrial | | |
| DL5-137 mg/kg with: | | | |
| Gemcitabine 1,000 | Renal Cell Carcinoma, Germ | 3 | 2 DLT: Grade 3 |
| mg/m² (3) | Cell, Fibrous Histocytoma | | AST elevation; |
| | | | Grade 4 |
| | | | Thrombocytopenia |
| 5FU 500 mg/m² (3) | Gastric, Cholangiosarcoma, | 3 | None |
| | Adenoid Cystic Carcinoma | | |
| Docetaxel 30 mg/m² | Still Enrolling | | |

The table below contains the adverse events reported with a frequency of 4% or greater.

TABLE 6
Dose Limiting Toxicities.

| | | Number and |
|---|---|---|
| | | Percentage of |
| Event | Grade | Occurrences |
| Elevated PT/PTT/INR | 2, 3* | 67 (26%) |
| Anemia | 2, 3 | 38 (15%) |
| Thrombocytopenia | 2, 3, 4* | 34 (13%) |
| Elevated AST | 2, 3 | 14 (6%) |
| Hypertriglyceridemia | 2, 4* | 15 (6%) |
| Fatigue | 2, 3 | 11 (4%) |
| Elevated PT/PTT/INR | 2, 3* | 67 (26%) |

TABLE 7
Coenzyme Q10 pharmacokinetics. a: n = 12; b: n = 11;
c: n = 9; d: n = 5; e: n = 4.

| | 220 mg/ | | | 342 mg/ |
|---|---|---|---|---|
| | kg/week | 274 mg/kg/week | | kg/week |
| Time | Arm 2, n = 13 | Arm 1, n = 3 | Arm 1, n = 6 | Arm 1, n = 5 |
| (hr) | Mean ± SD | Mean ± SD | Mean ± SD | Mean ± SD |
| 0 | 0 | 0 | 0 | 0 |
| 1 | 150 ± 54^{a} | 173 ± 36 | 188 ± 46 | 289 ± 59 |
| 2 | 163 ± 66 | 175 ± 42 | 190 ± 38 | 297 ± 81 |
| 4 | 158 ± 57^{b} | 185 ± 51 | 181 ± 56^{d} | 304 ± 90 |
| 24 | 251 ± 155 | 261 ± 149 | 287 ± 189 | 463 ± 274 |
| 71.5 | 255 ± 199 | 390 ± 260 | 265 ± 188^{d} | 563 ± 188 |
| 73 | 227 ± 212^{a} | 329 ± 260 | 367 ± 313 | 514 ± 205 |
| 74 | 226 ± 193^{a} | 335 ± 242 | 387 ± 332 | 537 ± 219 |
| 96 | 348 ± 225^{c} | 416 ± 291 | 407 ± 195^{e} | 411 ± 189^{e} |
| 140 | 378 ± 244^{b} | 513 ± 213 | 517 ± 185^{e} | 695 ± 414^{e} |
| 142 | 358 ± 214 | 514 ± 260 | 528 ± 179^{e} | 699 ± 290 |
| 143.5 | 363 ± 221^{a} | 510 ± 259 | 560 ± 246 | 789 ± 161^{e} |
| 146 | 282 ± 207^{b} | 486 ± 254 | 460 ± 249^{d} | 679 ± 141^{e} |
| 148 | 250 ± 251^{c} | 380 ± 219 | 397 ± 230^{d} | 596 ± 143^{e} |

Identification of Candidate Biomarkers

Clinical data was displayed in a “patient dashboard” to facilitate analysis of the data. The automatically generated dashboard allowed the comprehensive visualization of demographics and clinical outcomes for each patient enrolled in the trial. An example of the patient dashboard is provided in FIGS. 40A-40D. For example, FIG. 40A shows a summary of demographic information and trial outcome for patient 02-014. FIG. 40B shows tumor size progression for patient 02-014 relative to time of enrollment. FIG. 40C shows lab measurements for Patient 02-014 for blood glucose (GLUC); hematocrit (HCT); aspartate transaminase (AST); and alanine transaminase (ALT) ratio. Patient 02-014 experienced Grade 2 Adverse Events while enrolled on the clinical trial, as shown in FIG. 40D. FIG. 40E shows FDG-PET scans before and after treatment with Coenzyme Q10.

Proteomic, metabolomic and lipidomic analysis was performed on the blood (plasma and buffy coat) and urine samples collected from the patients to determine changes in protein, metabolite and lipid levels before and after treatment, and to identify differences between the overall clinical benefit and no clinical benefit patient groups. Technology-specific pipelines were used to convert these raw measurements into processed data by (1) combining data collected at different time points; (2) removing variables that are measured infrequently; (3) removing systematic biases to ensure samples are comparable across batches; and (4) inferring the level of any variable that was not measured in a particular sample. Data processing reliability was ensured by quality control (QC) steps including: (1) testing if raw data files follow expected formatting, and (2) making intuitive visualizations that track each step of the omics data processing. To ensure traceability, all outputs from the quality control were written to a central log file. The processed molecular features were made actionable by means of a Master File, which defines the patient and time point from which each sample was collected.

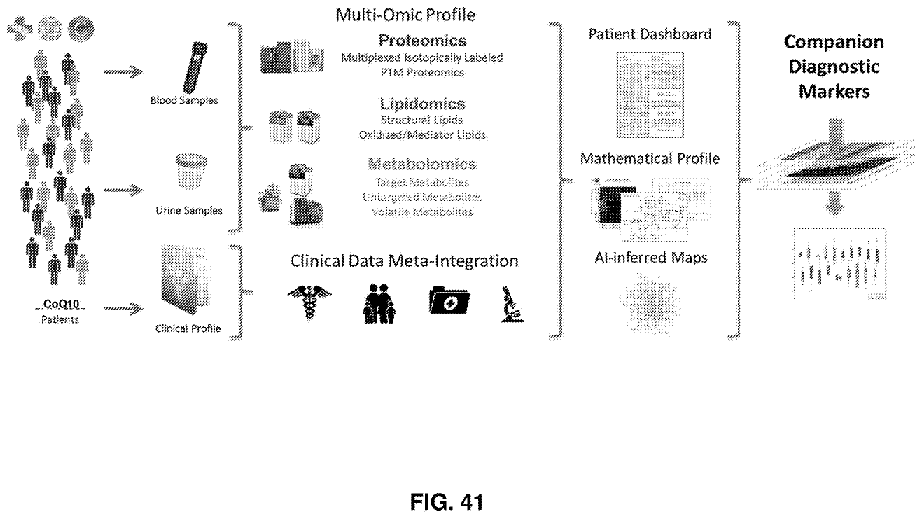

The processed data was then integrated with the clinical data described above. The resulting database included demographics, treatments, disease status, tumor size measurements, adverse events, lab measurements, clinical outcome, and pharmacokinetics data, proteomics, lipidomics, and metabolomics collected across time for all patients enrolled in the trial. This integrated data was used to create patient dashboards, mathematical profiles, and AI-inferred Maps, which were then mined to identify candidate biomarkers. Overviews of the analytics process are provided in FIG. 41 and in FIG. 4 described above.

For example, molecular features measured prior to treatment which were capable of differentiating overall clinical benefit patients from no clinical benefit patients were identified using three types of analysis, specifically, Bayesian network analysis, statistical analysis, and machine learning. Differences in the levels of several proteins, lipids and metabolites were identified between the patient groups during a sustained period following the trial start. Molecular signatures of response and safety were derived from the integrated omics and artificial intelligence (AI) profiling of the Interrogative Biology® platform. Machine learning was used to identify multi-omic variables that can predict if a sample (patient) belongs to the overall clinical benefit or no clinical benefit group.

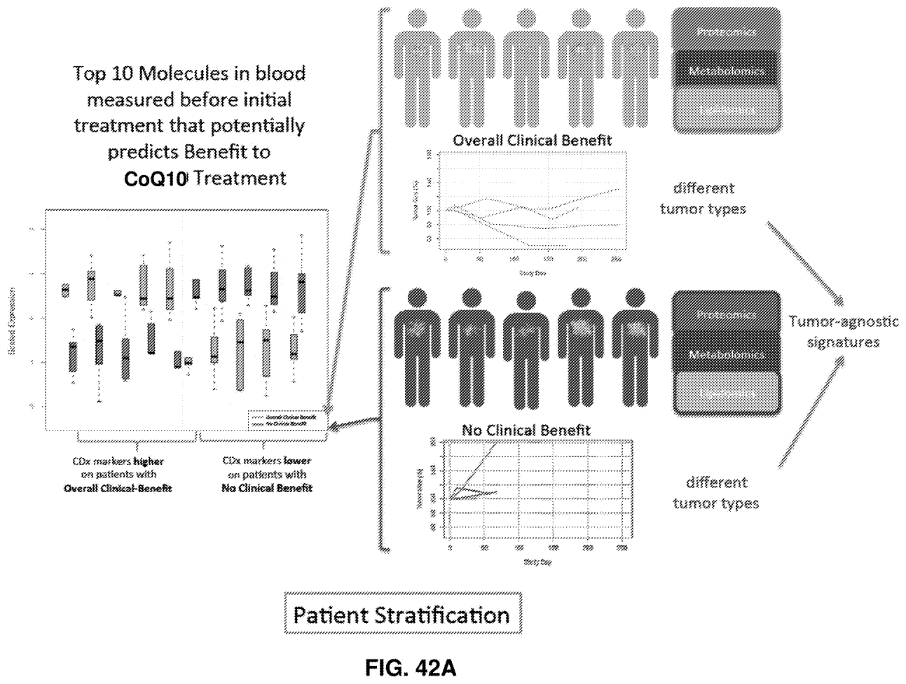

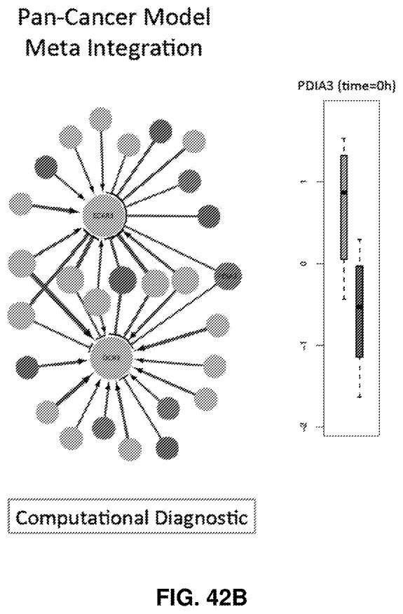

Biomarker candidates correlating with favorable clinical response and safety were identified. For example, FIG. 42A shows the top ten molecules in blood measured before initial Coenzyme Q10 treatment that may potentially predict the efficacy of Coenzyme Q10 treatment. pK levels of Coenzyme Q10 were a driver of favorable response. These molecular correlates were independent of tumor type and prior therapy, indicating a broad anti-tumor effect of Coenzyme Q10. Novel multi-omic panels could stratify response before and 24 hours post treatment with AUC>0.85.

Protein disulfide-isomerase A3 (PDIA3) is one candidate biomarker that was identified in this analysis. See FIG. 42B. Bayesian network analysis identified distinct differences in the bionetworks for PDIA3 between the overall clinical benefit and no clinical benefit patient groups. Several additional candidate biomarkers were also identified which exhibited quantitative differences between overall clinical benefit and no clinical benefit patients before Coenzyme Q10 treatment. These markers may be used to identify subjects afflicted with solid tumors that are likely to be responsive to Coenzyme Q10 therapy. The analysis described above may also be used to identify candidate biomarkers that are predictive of adverse events potentially caused by Coenzyme Q10 treatment, or that would be predictive of Coenzyme Q10 pharmacokinetics (PK).

Analysis for Identification of Candidate Biomarkers

A description of the slicing of the merged data and the analysis of the sliced data sets is described below.

The merged patient data was sliced in multiple slicing steps. A sliced data set including data from all patients was produced. The clinical output data was analyzed to identify overall clinical benefit and no clinical benefit patients. The merged data was sliced into a sliced data set including data from patients identified as exhibiting an overall clinical benefit in response to the treatment, and a sliced data set including data from patients identified as exhibiting no clinical benefit in response to the treatment.

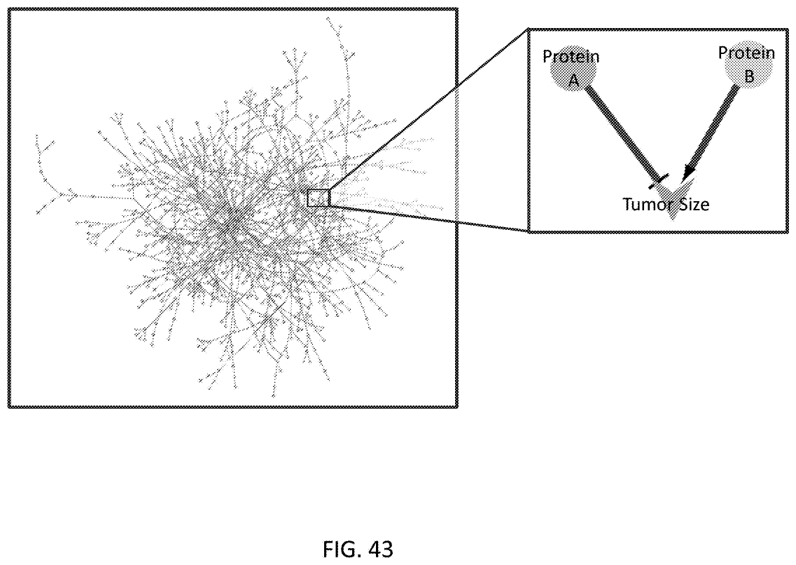

A Bayesian causal relationship network was generated from the sliced data set for all patients. Topological analysis of the Bayesian causal relationship network was used to identify potential regulators of tumor size, as schematically depicted in FIG. 43. The potential regulators of tumor size were compiled in a list.

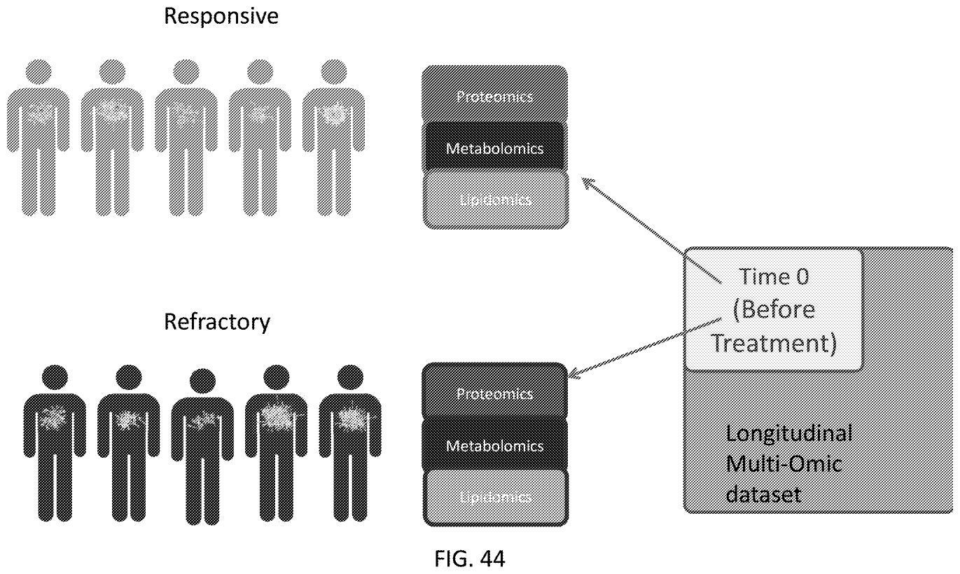

Molecular profile data corresponding to time zero (before treatment) was selected and sliced data sets for overall clinical benefit and no clinical benefit patients at time zero were prepared, as schematically depicted in FIG. 44.

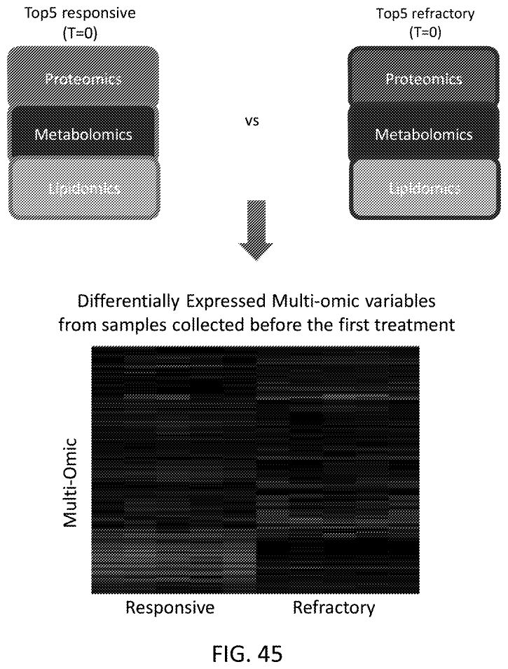

The time zero sliced data sets were statistically analyzed to identify components of the molecular profile that were differently expressed in the overall clinical benefit and no clinical benefit patients, as schematically depicted in FIG. 45.

Machine learning methods were employed to identify multi-omic variables based on the time zero sliced data to predict if a patient belongs to the overall clinical benefit or no clinical benefit group. The machine learning methods yielded a list of potential response predictors.

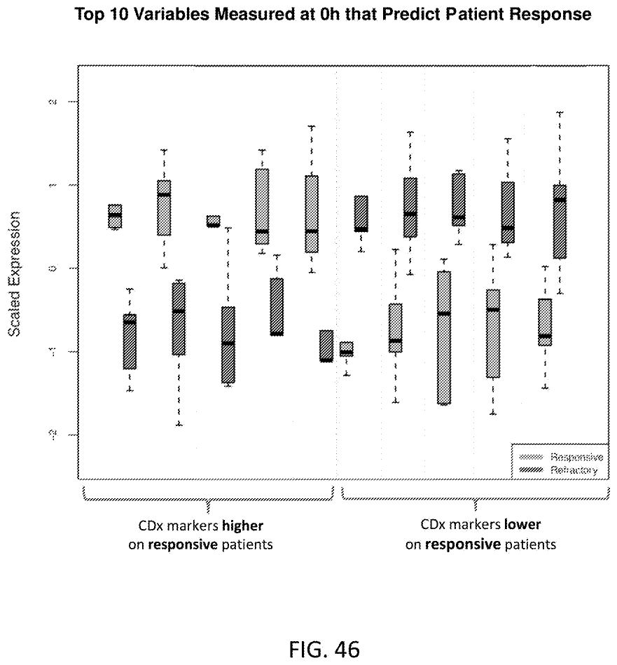

The regulators of tumor size from AI-based Bayesian network analysis, the time zero differently expressed molecular profile variables from statistical analysis, and the list of potential response predictors from the machine learning methods were used to identify biomarkers that may be measured at any time prior to therapy or after the trial begins to predict patient outcome (CDx). Specifically, the variables appearing on the overlap of the list of regulators of tumor size with the list of differently expressed molecular profile variables and the list of potential response predictors were identified as the companion diagnostics to predict patient outcome. FIG. 46 is a graph showing expression of these CDx markers in overall clinical benefit and no clinical benefit patients.

### EXAMPLE 2

### Identification of Candidate Biomarkers in a Phase 1 a/b Clinical Trial of CoQ10 for Treatment of Patients with Solid Tumors

Example 2 includes an analysis of candidate biomarkers in a Phase I clinical trial of CoQ10 for treatment of patients with solid tumors employing the CTAW 400 described above with respect to FIG. 4. Example 1 was based on a preliminary analysis of data obtained from some of the same patients in the same clinical trial; however, Example 2 is based on a larger number of patients, includes additional data, and incorporates additional analysis.

Trial Design

The trial was conducted for 36 months for patients with solid tumors at Weill Cornell University Medical Center, Palo Alto Medical Foundation and MD Anderson Cancer Center. This is a Phase 1 a/b clinical trial of a standard 3 +3 dose escalation design. The primary purpose of the trial was to determine the maximum tolerated dose and assess the safety and tolerability of CoQ10 alone and in combination with chemotherapy when administered as a 114 hour intravenous infusion. The secondary objective was to evaluate plasma pharmacokinetics and estimate renal clearance of CoQ10 mono and combination therapies.

Patients were routed to either Arm 1 (monotherapy, 45 patients) or Arm 2 (CoQ10 in combination with chemotherapy, 120 patients). All patients received 2 consecutive 72-hour infusions of CoQ10 on days 1, 4, 8, 11, 15, 18, 22, and 25 of each 28 day cycle. Patients were monitored for a minimum of 8 hours at the first infusion. The tumor sizes were measured using CT or MRI scans at the end of cycle 2 and every 2 cycles after that. Response to CoQ10 was measured by Response Evaluation Criteria in Solid Tumors (RECIST).

Patients that experienced no unacceptable toxicity or disease progression received additional 28 day cycles for up to 1 year on either arm. Selected patients on Arm 1 who progress were elected to continue with CoQ10 in addition to chemotherapy. Once a dose level of CoQ10 was evaluated and the CRC has determined this dose is safe, Arm 2, Cohort 1 was open to patient accrual. These patients received either gemcitabine, 5-FU or docetaxel in combination with CoQ10. Cycle 1 was CoQ10 administered twice weekly on Tuesday and Friday, with chemotherapy on Monday for six weeks. Cycles 2-12 were subsequently 4 weeks in duration. Response was assessed after Cycle 2 and every 2 Cycles thereafter. Patients originally on Arm 1 who progressed were transferred to Arm 2 if eligible, and received 4 weeks of treatment. Patients who progressed on combination therapy switched their chemotherapy component, or received CoQ10 alone. Once the maximum tolerated dose was established for both mono and combination therapies, an expansion cohort of patients were enrolled (12-15 patients for monotherapy and 10 patients each per combination therapy).

Pharmacokinetic/Pharmacodynamic (PK/PD) modeling

Blood samples were collected during each Cycle of mono and combination therapy. Urine samples were collected only during Cycle 1. A PET scan was performed within 2 weeks prior to starting CoQ10 and after 2 weeks of CoQ10 treatment. Arm 1 patients were scanned again at 8 weeks of treatment, and Arm 2 patients were scanned at 10 weeks of treatment. Five core biopsies were performed at baseline and at the end of week 2. Patients who cross over to Arm 2 also had the PET scans and biopsies within 2 weeks of starting CoQ10 and at week 3.

Drugs, Dose and Mode of Administration

CoQ10 nanosuspension injection (40mg/ml) was administered intravenously over 144 hours at the starting dose of 66 mg/kg. Each patient received 2 consecutive 48 hours infusions per week during each 28 day Cycle. The dose could be escalated 25% until maximum tolerate dose was reached. Once a safe CoQ10 dose was reached, Arm 2 opened for enrollment, and patients received CoQ10 at the confirmed dose and chemotherapy once per week with either Gemcitabine (600mg/m²), 5-FU (350 mg/m²) with leucovorin (100 mg/m²), or Docetaxel (20 mg/m²).

Using CTAW with Trial Data to identify candidate biomarkers

Patients enrolled in the CoQ10 solid tumor clinical trial had plasma, urine, and tissue samples subjected to multi-omic profiling to provide a high-dimensional view of their biology during their time on therapy. The CTAW 400, described above with respect to FIG. 4, performed all steps of data analysis beginning with data processing and ending with candidate diagnostic biomarker identification in a reliable, automated manner. Having organized the data analysis workflow into a pipeline enabled a user to produce deliverables as additional subjects were enrolled and additional clinical information became available.

For each patient, samples for obtaining pharmacokinetic values were obtained at the same time points (e.g., on the same days) as samples for obtaining molecular profile values so that no interpolation of pharmacokinetic values was needed to match the pharmacokinetic data to time points for the molecular profile data.

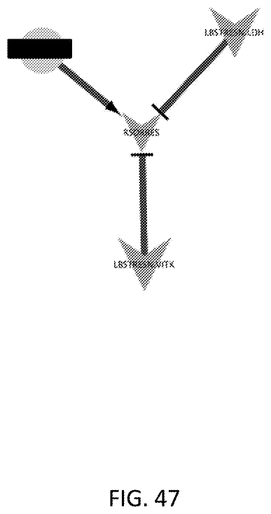

As described herein, the data collected during the trial was processed according to the CTAW 400. One of the steps of the CTAW 400 was slicing the data to generate networks using Bayesian learning. Drivers of key clinical variables were be harvested from the AI networks generated by the CTAW. Based on this example trial, the workflow generated 137 networks that contain drivers of patient outcome variables (TRORRES, TRPCT, and RSORRES) illustrated in Table 9 below. Here, drivers are defined as nodes serving as parents to patient outcome variables, which as bottom variables are constrained from having connections to child nodes (see FIG. 47).

Table 8 below illustrates various data slices created from the data collected during this trial, and the number of networks generated from the data slices. RSORRES refers to the tumor response by the RECSIT criteria. TRORRES is the geometric mean of patient tumor sizes measured at a particular time. TRPCT is relative tumor size such that each patient has a tumor size of 100% at trial enrollment.

Exemplary data slices are listed in Table 8 below.

TABLE 8
Data Sliced According to Phenotypic Variables.

| | | | Limited to | Limited |
|---|---|---|---|---|
| Slice | | | Individual | to |
| Variable(s) | Slice Example | Description | Patient? | Cycle 1? |
| RSORRES | RSORRES = | Tumor response | No | No |
| | SD | was stable disease | | |
| Patient ID | Patient | All observations | Yes | No |
| | ID = 01-001 | from patient 01- | | |
| | | 001 | | |
| None | Full | All observations | No | No |
| Treatment | 5-FU = True | Observations | No | No |
| | | from patients who | | |
| | | were assigned to | | |
| | | treatment arm 5- | | |
| | | FU | | |
| Adverse Event | Toxicity | Observations | No | No |
| | Grade = 1 | made during | | |
| | | which patient | | |
| | | experienced | | |
| | | adverse event of | | |
| | | toxicity grade 1 | | |
| Cycle and | Cycle = 1 & | Observations | No | Yes |
| Treatment | 5-FU = True | made during cycle | | |
| | | 1 from patients | | |
| | | who were | | |
| | | assigned to | | |
| | | treatment arm | | |
| | | including 5-FU | | |
| Cycle and | Cycle = 1 & | Observations | No | Yes |
| Infusion | Infusion | made during cycle | | |
| Schedule | Schedule = 144 | 1 from patients | | |
| | Hour | who were | | |
| | | assigned to the | | |
| | | 144 hour infusion | | |
| | | schedule | | |
| Cycle and | Cycle = 1 & | Observations | Yes | Yes |
| Patient ID | Patient | made during | | |
| | ID = 01-001 | Cycle 1 for | | |
| | | patient 01-001 | | |
| Cycle | Cycle = 1 | All observations | No | Yes |
| | | made during | | |
| | | Cycle 1 | | |

TABLE 9
AI networks harvested to identify drivers of key
clinical output variables.

| | Number | | | |
|---|---|---|---|---|
| | of | TRORRES | TRPCT | RSORRES |
| Data Slice | Networks | Present? | Present? | Present? |
| Patient Response | 3 | Yes | Yes | No |
| (RECIST) | | | | |
| Patient ID | 42 | Yes | Yes | Yes |
| Full | 1 | Yes | Yes | Yes |
| Treatment | 8 | Yes | Yes | Yes |
| Adverse Event | 40 | Yes | Yes | Yes |
| Treatment during | 8 | Yes | Yes | No |
| Cycle 1 | | | | |
| Infusion Schedule | 2 | Yes | Yes | No |
| during Cycle 1 | | | | |
| Patient ID during | 32 | Yes | Yes | No |
| Cycle 1 | | | | |
| Full Cycle 1 | 1 | Yes | Yes | No |

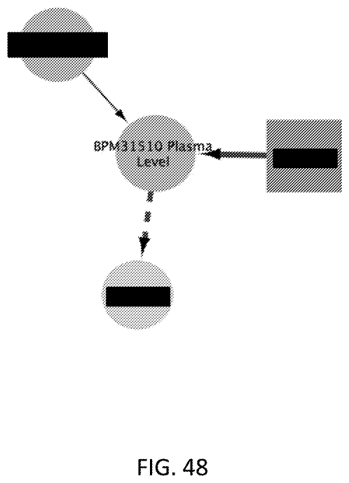

Similarly, insights into the mechanisms of action (MOA) of CoQ10 were found from AI networks generated by the CTAW. These insights manifested in AI networks as causal relationships between the plasma levels of CoQ10 and downstream molecular features. MOA insights were harvested from patient data collected during Cycle 1, in which PK measurements were available (Table 10). An example of MOA from the network learned from Cycle 1 data from patients infused on a 96-hour schedule is shown in FIG. 48.

TABLE 10
AI networks containing the plasma levels of CoQ10 were
harvested to gain insight into CoQ10 MOA.

| | Number of | CoQ10 Plasma |
|---|---|---|
| Data Slice | Networks | Level Present? |
| Treatment during Cycle | 8 | Yes |
| 1 | | |
| Infusion Schedule | 2 | Yes |
| during Cycle 1 | | |
| Patient ID during Cycle | 32 | Yes |
| 1 | | |
| Full Cycle 1 | 1 | Yes |

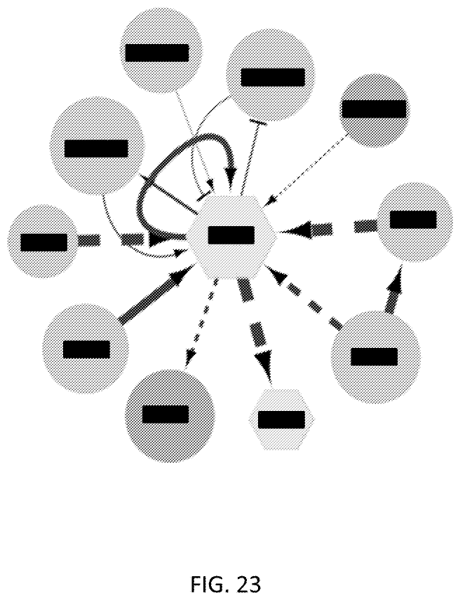

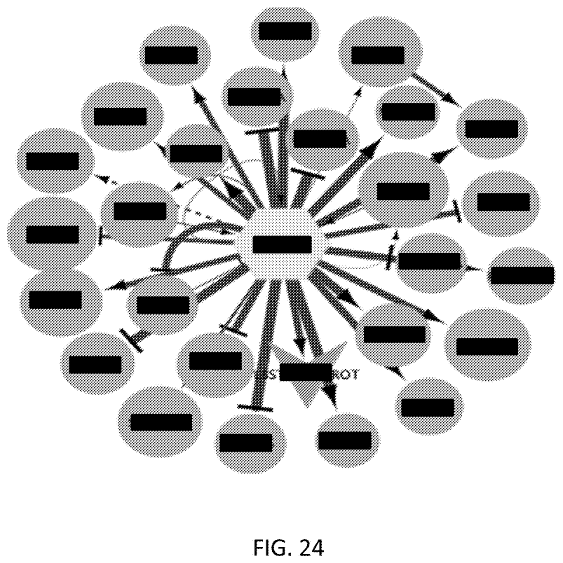

Exemplary networks generated from the data obtained from this example trial are illustrated in FIGS. 22-27. Subnetworks showing key outcome drivers are shown in FIGS. 23, 24, 33 and 34. A differential network (delta) based on a comparison of a network generated from data from patients who experienced severed adverse and a network generated from data from patients who did not experience the severed adverse effect was generated and is shown in FIG. 34.

Regression analysis as described above with respect to FIG. 4 was used to identify statistically significant differentially expressed variables for prediction of responsivity and for prediction of efficacy. Statistically significant differentially expressed variables for prediction of severe adverse effects prior to treatment were determined, as shown in FIG. 35.

Machine learning employing regression with an elastic net penalty coupled with bootstrap resampling was used to identify potential biomarkers, specifically CDx markers, from a group of possible biomarkers, specifically candidate CDx markers, including outcome drivers identified from AI-network analysis and the differentially expressed variables. The elastic net parameters and results of the machine learning are shown in Table 11 below. Table 11 lists the Top 10 robust features measured at time zero between patients who experienced grade three or higher adverse events, and patients who did not. Robustness was defined by the percent bootstrap resamples present.

TABLE 11
Parameters and results from elastic net penalized
regression with bootstrap resampling.

| | | | | % Bootstrap |
|---|---|---|---|---|
| | | | | Resamples |
| ID | α | λ | Deviance | Present |
| Redacted | 0.05 | 0.082 | 0.277 | 0.998 |
| Redacted | 0.05 | 0.082 | 0.277 | 0.998 |
| Redacted | 0.05 | 0.082 | 0.277 | 0.998 |
| Redacted | 0.05 | 0.082 | 0.277 | 0.996 |
| Redacted | 0.05 | 0.082 | 0.277 | 0.996 |
| Redacted | 0.05 | 0.082 | 0.277 | 0.996 |
| Redacted | 0.05 | 0.082 | 0.277 | 0.994 |
| Redacted | 0.05 | 0.082 | 0.277 | 0.994 |
| Redacted | 0.05 | 0.082 | 0.277 | 0.994 |
| Redacted | 0.05 | 0.082 | 0.277 | 0.994 |

Scaled expression values for CDx markers for measurements prior to therapy that predicted responsivity are shown in FIG. 31.

Scaled expression values for CDx markers for measurements prior to therapy that predicted severe adverse effects are shown in FIG. 32.

Expression levels of the top 10 CDx markers for overall clinical benefit and no clinical benefit are shown in FIG. 46.

Systems for Implementing Methods

Certain embodiments are described herein as including logic or a number of components, modules, or mechanisms. Modules may constitute either software modules (e.g., code embodied on a machine-readable medium or in a transmission signal) or hardware modules. A hardware module is a tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA), an application-specific integrated circuit (ASIC), or a Graphics Processing Unit (GPU)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired) or temporarily configured (e.g., programmed) to operate in a certain manner and/or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor-implemented. For example, at least some of the operations of a method may be performed by one or processors or processor-implemented modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., APIs).

Example embodiments may be implemented in digital electronic circuitry, or in computer hardware, firmware, software, or in combinations of them. Example embodiments may be implemented using a computer program product, for example, a computer program tangibly embodied in an information carrier, for example, in a machine-readable medium for execution by, or to control the operation of, data processing apparatus, for example, a programmable processor, a computer, or multiple computers.

A computer program can be written in any form of programming language, including compiled or interpreted languages, and it can be deployed in any form, including as a stand-alone program or as a module, subroutine, or other unit suitable for use in a computing environment. A computer program can be deployed to be executed on one computer or on multiple computers at one site or distributed across multiple sites and interconnected by a communication network.

In example embodiments, operations may be performed by one or more programmable processors executing a computer program to perform functions by operating on input data and generating output. Method operations can also be performed by, and apparatus of example embodiments may be implemented as, special purpose logic circuitry (e.g., a FPGA or an ASIC).

The computing system can include clients and servers. A client and server are generally remote from each other and typically interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice. Below are set out hardware (e.g., machine) and software architectures that may be deployed, in various example embodiments.

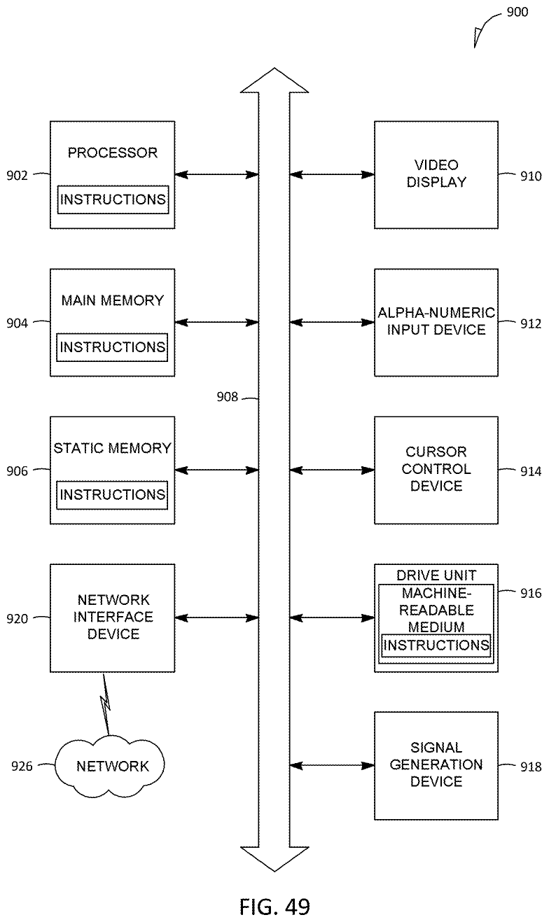

FIG. 49 is a block diagram of machine in the example form of a computer system 900 within which instructions, for causing the machine (e.g., device 110, 115, 120, 125; servers 130, 135; database server(s) 140; database(s) 130) to perform any one or more of the methodologies discussed herein, may be executed. In alternative embodiments, the machine operates as a standalone device or may be connected (e.g., networked) to other machines. In a networked deployment, the machine may operate in the capacity of a server or a client machine in a server-client network environment, or as a peer machine in a peer-to-peer (or distributed) network environment. The machine may be a personal computer (PC), a tablet PC, a set-top box (STB), a PDA, a cellular telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 900 includes a processor 902 (e.g., a central processing unit (CPU), a multi-core processor, and/or a graphics processing unit (GPU)), a main memory 904 and a static memory 906, which communicate with each other via a bus 908. The computer system 900 may further include a video display unit 910 (e.g., a liquid crystal display (LCD), a touch screen, or a cathode ray tube (CRT)). The computer system 900 also includes an alphanumeric input device 912 (e.g., a physical or virtual keyboard), a user interface (UI) navigation device 914 (e.g., a mouse), a disk drive unit 916, a signal generation device 918 (e.g., a speaker) and a network interface device 920.

The disk drive unit 916 includes a machine-readable medium 922 on which is stored one or more sets of instructions and data structures (e.g., software) 924 embodying or used by any one or more of the methodologies or functions described herein. The instructions 924 may also reside, completely or at least partially, within the main memory 904, static memory 906, and/or within the processor 902 during execution thereof by the computer system 900, the main memory 904 and the processor 902 also constituting machine-readable media.

While the machine-readable medium 922 is shown in an example embodiment to be a single medium, the term “machine-readable medium” may include a single medium or multiple media (e.g., a centralized or distributed database, and/or associated caches and servers) that store the one or more instructions or data structures. The term “machine-readable medium” shall also be taken to include any tangible medium that is capable of storing, encoding or carrying instructions for execution by the machine and that cause the machine to perform any one or more of the methodologies of the present invention, or that is capable of storing, encoding or carrying data structures used by or associated with such instructions. The term “machine-readable medium” shall accordingly be taken to include, but not be limited to, solid-state memories, and optical and magnetic media. Specific examples of machine-readable media include non-volatile memory, including by way of example, semiconductor memory devices (e.g., Erasable Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 924 may further be transmitted or received over a communications network 926 using a transmission medium. The instructions 924 may be transmitted using the network interface device 920 and any one of a number of well-known transfer protocols (e.g., HTTP). Examples of communication networks include a LAN, a WAN, the Internet, mobile telephone networks, Plain Old Telephone (POTS) networks, and wireless data networks (e.g., WiFi and WiMax networks). The term “transmission medium” shall be taken to include any intangible medium that is capable of storing, encoding or carrying instructions for execution by the machine, and includes digital or analog communications signals or other intangible media to facilitate communication of such software.

Although the present invention has been described with reference to specific example embodiments, it will be evident that various modifications and changes may be made to these embodiments without departing from the broader spirit and scope of the invention. Accordingly, the specification and drawings are to be regarded in an illustrative rather than a restrictive sense.

It will be appreciated that, for clarity purposes, the above description describes some embodiments with reference to different functional units or processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

Although an embodiment has been described with reference to specific example embodiments, it will be evident that various modifications and changes may be made to these embodiments without departing from the broader spirit and scope of the invention. Accordingly, the specification and drawings are to be regarded in an illustrative rather than a restrictive sense. The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be used and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended; that is, a system, device, article, or process that includes elements in addition to those listed after such a term in a claim are still deemed to fall within the scope of that claim. Moreover, in the following claims, the terms “first,” “second,” and “third” and so forth are used merely as labels, and are not intended to impose numerical requirements on their objects.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

1. A method comprising:
processing molecular profile data for each subject in a plurality of subjects, the molecular profile data for each subject comprising one or more of proteomics, metabolomics, lipidomics, genomics, transcriptomics, microarray and sequencing data generated from analysis of a plurality of samples obtained from the subject; the plurality of samples for each subject including samples obtained before, during, and/or after administration of an agent to the subject;
processing clinical records data for each of the plurality of subjects, the clinical records data for each subject including data based on one or both of samples obtained from the subject and measurements made of the subject before, during, and/or after administration of the agent, the clinical records data comprising clinical outcome data;
integrating the processed molecular profile data and the processed clinical records data for the plurality of subjects and storing in a database as merged data;
selecting two or more subsets of the merged data using one or more criteria based on the clinical records data to generate two or more selected data sets; and
analyzing one or more of the selected data sets to identify one or more potential biomarkers for a clinical outcome related to administration of the agent.

2. The method of claim 1, further comprising, administering the agent to the plurality of subjects.

3. The method of claim 1, further comprising, for each subject, analyzing the plurality of samples obtained from the subject to obtain the molecular profile data.

4. The method of claim 1, wherein the clinical records data further comprises one or more of pharmacokinetics data, medical history data, laboratory test data, data from a mobile wearable device, and demographic information regarding the subject.

5. (canceled)

6. The method of claim 1, wherein the one or more selected data sets are analyzed using one or more of statistical methods, machine learning methods, and artificial intelligence methods to identify the one or more potential biomarkers for the clinical outcome related to administration of the agent.

7. (canceled)

8. The method of claim 1, wherein analyzing one or more of the selected data sets to identify the one or more potential biomarkers for the clinical outcome related to administration of the agent comprises:
generating one or more causal relationship networks based on one or more of the selected data sets; and
analyzing the generated one or more causal relationship networks to identify nodes corresponding to one or more outcome drivers.

9. The method of claim 8, wherein analyzing the generated causal relationship networks to identify nodes corresponding to the one or more outcome drivers includes identifying as outcome drivers variables corresponding to nodes connected to the clinical outcome in one or more of the generated causal relationship networks by relationships having a degree of connection equal to or less than n, wherein n is 10 or 9 or 8 or 7 or 6 or 5 or 4 or 3 or 2 or 1.

10.-11. (canceled)

12. The method of claim 8, wherein analyzing the generated causal relationship networks to identify nodes corresponding to the one or more outcome drivers includes analysis of network topology features of the one or more generated causal relationship networks.

13. The method of claim 8, wherein the generated two or more selected data sets comprise a first plurality of selected data sets each corresponding to a subject that exhibited the clinical outcome and a second plurality of selected data sets each corresponding to a subject that did not exhibit the first clinical outcome;
wherein generating the one or more causal relationship networks based on one or more of the selected data sets includes:
generating a first plurality of causal relationship networks each based on one of the first plurality of selected data sets corresponding to subjects that exhibited the clinical outcome, and
generating a second plurality of causal relationship networks each based on one of the second plurality of selected data sets corresponding to subjects that did not exhibit the clinical outcome; and
wherein analyzing the generated causal relationship networks to identify nodes corresponding to one or more outcome drivers includes:
identifying one or more first commonalities among first plurality of causal relationship networks,
identifying one or more second commonalities among the second plurality of causal relationship networks, and
comparing the first commonalities and the second commonalities to identify the one or more outcome drivers.

14. The method of claim 8, wherein the generated two or more selected data sets comprise a first selected data set including data corresponding to one or more subjects that exhibited the clinical outcome and a second selected data set including data corresponding to one or more subjects that did not exhibit the clinical outcome;
wherein generating the one or more causal relationship networks based on at least some of the selected data sets includes:
generating a first causal relationship network based on the first selected data set corresponding to subjects that exhibited the clinical outcome, and
generating a second causal relationship network based on the second selected data set corresponding to subject that did not exhibit the clinical outcome, and
wherein the one or more outcome drivers are identified based on a comparison of the first causal relationship network to the second causal relationship network.

15. The method of claim 14, wherein the comparison of the first causal relationship network to the second causal relationship network includes generation of a differential causal relationship from the first causal relationship network and the second causal relationship network, and wherein the one or more outcome drivers are identified from the generated differential causal relationship network.

16.-17. (canceled)

18. The method of claim 8, wherein the generated two or more selected data sets includes a first selected data set including data from subjects that exhibited the clinical outcome and a second sliced data including to data from subjects that did not exhibit the clinical outcome; and
wherein analyzing one or more of the selected data sets to identify one or more potential biomarkers for a clinical outcome related to administration of the agent further comprises identifying one or more variables differentially expressed between first selected data set and the second selected data set at a statistically significant level.

19. The method of claim 18, wherein the first selected data set and the second selected data set correspond to the same time point or the same range of time points relative to a time of administration of an agent.

20. The method of claim 18, wherein identifying the one or more variables differentially expressed between first selected data set and the second selected data set at a statistically significant level employs a two-sample t-test or limma methodology or performing a regression analysis.

21. (canceled)

22. The method of claim 18, wherein analyzing one or more of the selected data sets to identify one or more potential biomarkers for a clinical outcome related to administration of the agent further comprises:
employing machine learning to analyze the identified outcome drivers and the one or more differentially expressed variables as possible biomarkers and, based on the analysis, selecting a subset of the possible biomarkers as the one or more potential biomarkers, wherein the machine learning penalizes possible biomarkers that are strongly correlated with other possible biomarkers and rewards possible biomarkers based on a level of correlation with the clinical outcome, thereby identifying one or more potential biomarkers for the clinical outcome.

23. The method of claim 22, wherein the machine learning employed to analyze the possible biomarkers applies logistic regression with the elastic net penalty.

24. The method of claim 1, wherein integrating the processed molecular profile data and the processed clinical records data for the plurality of subjects and storing in the database as merged data comprises storing the merged data in a master file that includes a subject identification and a time associated with each sample.

25. The method of claim 1, wherein linear interpolation is used to determine interpolated values of at least some clinical records data at times corresponding to those associated with molecular profile samples.

26. The method of claim 8, further comprising:
generating an in silico computational diagnostic patient map for determination of a subject response from analysis of topological features of the generated causal relationship networks.

27. (canceled)

28. The method of claim 1, wherein the one or more potential biomarkers are potential biomarkers for agent efficacy or for an adverse event.

29. The method of claim 1, wherein the method is a method for identifying one or more potential biomarkers for efficacy of the agent in treatment of a disease or a disorder or for the occurrence of an adverse event related to administration of the agent.

30. (canceled)

31. The method of claim 1, wherein the method is a method for patient stratification; and wherein the method further comprises employing the one or more potential biomarkers for patient stratification.

32. The method of claim 1, wherein the one or more potential biomarkers are employed for patient stratification to determine whether or not to treat a patient using the agent.

33. The method of claim 1, wherein the method is a method for patient stratification;
wherein the administration of an agent to the plurality of subjects occurs during a clinical trial for the agent; and
wherein the method further comprises employing the identified one or more potential biomarkers for patient stratification during a subsequent clinical trial of the agent or during a subsequent stage of the same clinical trial of the agent.

34. The method of claim 33, wherein the one or more potential biomarkers are used for patient stratification to determine which patients are enrolled in the subsequent clinical trial or to determine the patients that receive the agent in the subsequent clinical trial.

35. (canceled)

36. The method of claim 1, wherein the one or more criteria for selecting two or more subsets of the merged data includes a phenotypic classification or includes clinical outcome data or includes data regarding whether a subject experienced an adverse event during or after administration of the agent.

37.-38. (canceled)

39. The method of claim 1, wherein the agent is intended for treatment of a disease or disorder and wherein the one or more criteria for selecting two or more subsets of the merged data includes data regarding responsiveness of the subject to the treatment.

40. The method of claim 1, wherein the selected two or more subsets of the merged data include a selected data set for each individual subject.

41. The method of claim 1, wherein the two or more selected data sets comprise a selected data set including the merged data from all of the plurality of subjects.

42. The method of claim 1, wherein the one or more samples for each subject comprise one or more of blood, tissue, and urine samples.

43. (canceled)

44. The method of claim 1, wherein the molecular profile data for each subject comprises two or more of proteomics, metabolomics, lipidomics, genomics, transcriptomics, microarray and sequencing data.

45.-47. (canceled)

48. The method of claim 1, wherein the clinical outcome data comprises data regarding a state or status of a disease or a disorder.

49. The method of claim 1, wherein the agent is an agent for treatment of a disease or disorder and wherein the clinical outcome data comprises data indicating whether a subject was responsive or refractory in response to treatment with the agent.

50. The method of claim 1, wherein the clinical outcome data comprises data regarding an adverse event occurring during or after administration of the agent.

51. The method of claim 1, further comprising:
processing the merged data by reconciling duplicated clinical records data and resolving discrepancies.

52. The method of claim 1, further comprising:
filtering the merged data to remove molecular data for which corresponding clinical records data is missing.

53. The method of claim 1, wherein processing molecular profile data for each subject further comprises:
merging the molecular profile data collected at different time points over the course of the treatment for the plurality of subjects;
filtering the molecular profile data to remove infrequently measured variables;
normalizing the molecular profile data; and
imputing any variable not measured for a particular subject of the plurality of subjects.

54. The method of claim 1, wherein the agent is intended for treatment of cancer.

55. The method of claim 54, wherein the clinical outcome data includes tumor size measurements or comprises data from functional imaging of a tumor.

56. (canceled)

57. The method of claim 54, wherein analyzing one or more of the selected data sets to identify one or more potential biomarkers for a clinical outcome related to administration of the agent comprises generating a Bayesian causal relationship network for each of the one or more selected data sets; and
wherein the method further comprises comparing the generated Bayesian causal relationship networks from selected data sets from subjects with a Bayesian causal relationship network generated based on data obtained from an in vitro model of cancer.

58. The method of claim 1, further comprising generating a subject-specific profile, the subject-specific profile comprising:
a graphical representation of demographic information for the subject; and
a graphical representation of outcome information for the subject.

59. The method of claim 58, wherein the graphical representation of outcome information for the subject comprises:
a graphical representation of adverse event information for the subject; and
a graphical representation of information regarding responsivity to the agent.

60. The method of claim 1, wherein some or all of the subjects in the plurality of subjects are afflicted with a disorder.

61. The method of claim 60, wherein the disorder is selected from the group consisting of cancer, diabetes and cardiovascular disease.

62.-63. (canceled)

64. The method claim 1, wherein, for each subject, the clinical records data includes pharmacokinetic data from samples obtained at the same time points as samples for molecular profile data were obtained.

65. The method of claim 1, further comprising, for each patient, obtaining the plurality of samples for molecular profile data at a plurality of time points and obtaining samples for pharmacokinetic data at the same plurality of time points.

66. The method of claim 54, wherein the method is a method of identifying one or more biomarkers for the clinical outcome related to administration of the agent, and wherein the identified one or more potential biomarkers are one or more biomarkers for the clinical outcome related to administration of the agent.

67. A system comprising:
a database;
a memory; and
a processor in communication with the memory, the processor comprising::
an omics module configured to process molecular profile data for each subject in a plurality of subjects, the molecular profile data for each subject comprising one or more of proteomics, metabolomics, lipidomics, genomics, transcriptomics, microarray and sequencing data generated from analysis of a plurality of samples obtained from the subject, the plurality of samples for each subject including samples obtained before, during, and/or after administration of an agent to the subject;
a clinical records module configured to process clinical records data for each of the plurality of subjects, the clinical records data for each subject including data based on one or both of samples obtained from the subject and measurements made of the subject before, during, and/or after administration of the agent, the clinical records data comprising clinical outcome data;
an integration module configured to integrate the processed molecular profile data and the processed clinical records data for the plurality of subjects and storing in the database as merged data;
a slicing module configured to select two or more subsets of the merged data using one or more criteria based on the clinical records data to generate two or more selected data sets; and
an analysis module configured to analyze one or more of the selected data sets to identify one or more potential biomarkers for a clinical outcome related to administration of the agent.

68.-129. (canceled)
